(19) United States
(12) Patent Application Publication
(10) Pub. No.: US 2017/0144998 A1
(43) Pub. Date: May 25, 2017

(54) ELASTASE INHIBITORS

(71) Applicant: The California Institute for Biomedical Research, La Jolla, CA (US)

(72) Inventors: Arnab K. CHATTERJEE, San Diego, CA (US); Manoj KUMAR, San Diego, CA (US); Peter G. SCHULTZ, La Jolla, CA (US)

(21) Appl. No.: 15/381,507

(22) Filed: Dec. 16, 2016

Related U.S. Application Data
(63) Continuation of application No. PCT/US15/37229, filed on Jun. 23, 2015.
(60) Provisional application No. 62/016,535, filed on Jun. 24, 2014.

(51) Int. Cl. C07D 405/14; C07D 401/14; C07D 213/82; C07D 207/16

(52) U.S. Cl. CPC C07D 405/14; C07D 207/16; C07D 401/14; C07D 213/82

(57) ABSTRACT

Described herein are compounds, compositions, and methods of their use for the treatment of a lung or intestinal disease.

### CROSS-REFERENCE

This application is a continuation of International Application No. PCT/US15/37229, filed Jun. 23, 2015, which claims the benefit of U.S. provisional application Ser. No. 62/016,535, filed Jun. 24, 2014, both of which are incorporated herein by reference in their entirety.

### BACKGROUND OF THE INVENTION

Chronic obstructive pulmonary disease (COPD) is a type of obstructive lung disease characterized by chronically poor airflow. COPD is a major cause of disability, and it's one of the leading causes of death in the United States. Currently, millions of people are diagnosed with COPD. The main symptoms include shortness of breath, cough, and sputum production. Airflow obstruction arising from the hypersecretion of mucous into the airways, a characteristic feature of COPD and other chronic lung diseases, is due, at least in part, to the stimulation of goblet cells by human neutrophil elastase (HNE). Therefore, elastase inhibitors, either as single agents or in combination with another agent, may be effective in treating COPD.

Inflammatory bowel disease (IBD) is a group of inflammatory conditions of the colon and small intestine. The major types of IBD are Crohn's disease and ulcerative colitis. IBD is one of the most prevalent gastrointestinal diseases, with an overall health care cost of more than $1.7 billion in the United States alone. It has been reported that neutrophil elastase activity is elevated in both colonic mucosa and blood in inflammatory bowel disease (IBD) patients, and that it can act as an aggravating factor in IBD. Therefore, elastase inhibitors, either as single agents or in combination with another agent, may be effective in treating IBD.

### SUMMARY OF THE INVENTION

In one aspect, provided herein are compounds of Formula (I), or pharmaceutically acceptable salts, solvates, polymorphs, prodrugs, metabolites, deuterides, N-oxides, stereoisomers, or isomers thereof:

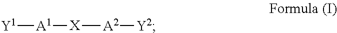

wherein:
A¹ and A² are independently selected from:

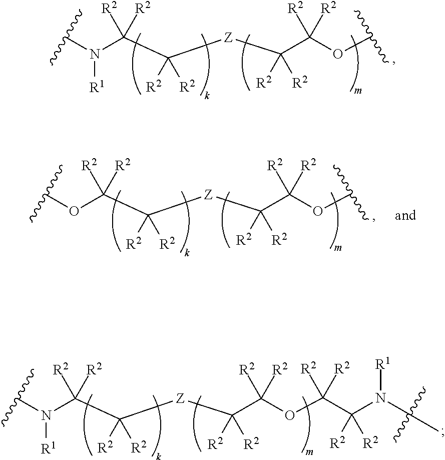

Y¹ and Y² are independently an elastase inhibitor;
X is alkylene, —C(O)—, —C(O)C(O)—,

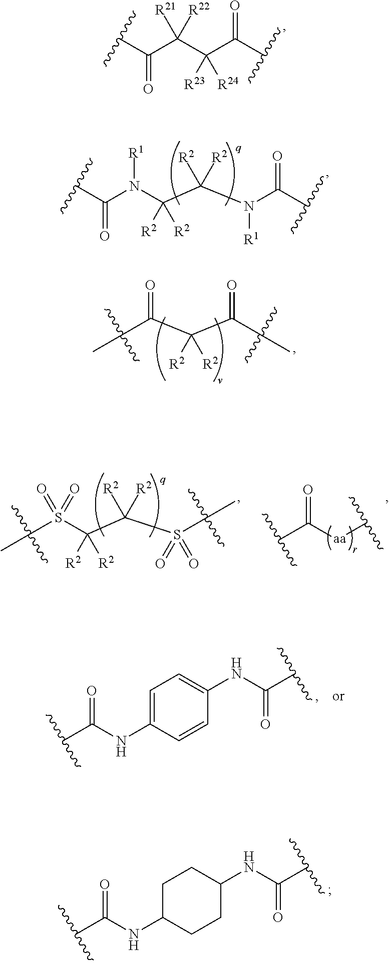

Z is O or CR²R²;
aa is

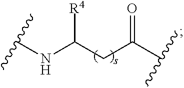

each R¹ is independently selected from H, alkyl, haloalkyl, arylalkyl, and heteroarylalkyl;
each R² is independently selected from H, halo, —CN, —SR¹, alkyl, cycloalkyl, haloalkyl, —OR¹, and —R⁴;
each R⁴ is independently selected from —CO₂R¹, -(alkylene)-(CO₂R¹), hydroxyalkyl, -(alkylene)(S(O)ₜ)(alkyl), -(alkylene)(NR⁵R⁵), and

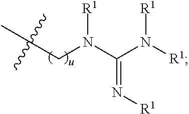

each R⁵ is independently selected from H, alkyl, haloalkyl, arylalkyl, heteroarylalkyl, aryl, and heteroaryl;
R²¹, R²², R²³, and R²⁴ are independently selected from H, halo, —CN, —SR¹, alkyl, cycloalkyl, haloalkyl, —NR¹R¹, and —OR¹;
each k is independently selected from 1, 2, 3, 4, 5, 6, 7, 8, 9, and 10;
each m is independently selected from 0, 1, 2, 3, 4, 5, 6, 7, 8, 9, and 10;
q is 1, 2, 3, 4, 5, 6, 7, 8, 9, or 10;
r is 3, 4, 5, 6, or 7;
each s is independently selected from 0, 1, 2, 3, and 4;
each t is independently selected from 0, 1, and 2;
each u is independently selected from 1, 2, 3, 4, 5, 6, 7, 8, 9, and 10; and
v is selected from 3, 4, 5, 6, 7, 8, 9, and 10.

In some embodiments described above or below of a compound of Formula (I), Y¹ and Y² are selected from sivelestat, alvelastat, DMP-777, and a derivative thereof. In some embodiments described above or below of a compound of Formula (I), Y¹ and Y² are independently selected from

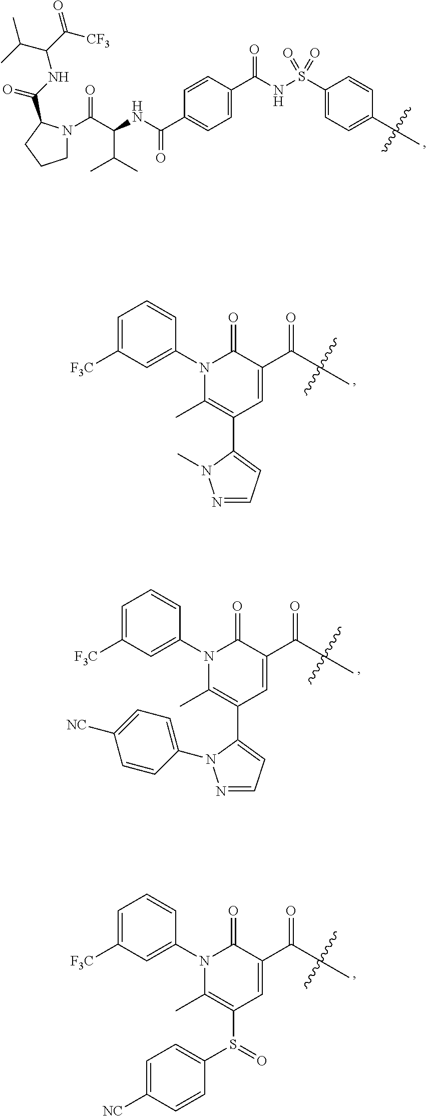 [SMILES: *c1ccc(S(=O)(=O)NC(=O)c2ccc(C(=O)N[C@H](C(=O)N3CCC[C@H]3C(=O)NC(C(=O)C(F)(F)F)C(C)C)C(C)C)cc2)cc1 ; *C(=O)c1cc(-c2ccnn2C)c(C)n(-c2cccc(C(F)(F)F)c2)c1=O ; *C(=O)c1cc(-c2ccnn2-c2ccc(C#N)cc2)c(C)n(-c2cccc(C(F)(F)F)c2)c1=O ; *C(=O)c1cc(S(=O)c2ccc(C#N)cc2)c(C)n(-c2cccc(C(F)(F)F)c2)c1=O]

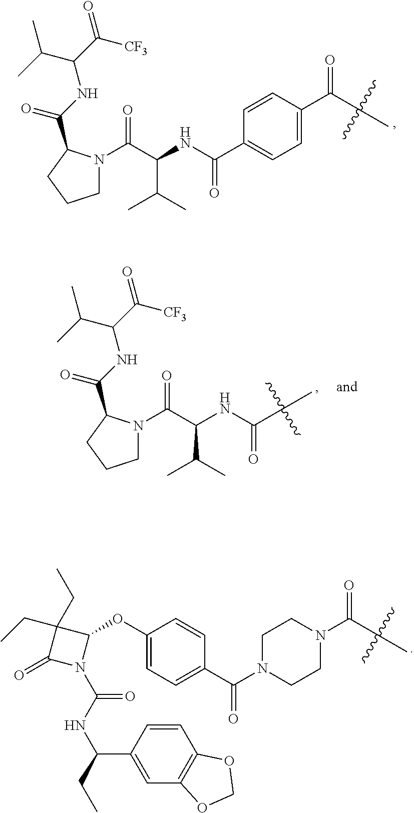 [SMILES: *C(=O)c1ccc(C(=O)N[C@H](C(=O)N2CCC[C@H]2C(=O)NC(C(=O)C(F)(F)F)C(C)C)C(C)C)cc1 ; *C(=O)N[C@H](C(=O)N1CCC[C@H]1C(=O)NC(C(=O)C(F)(F)F)C(C)C)C(C)C ; *C(=O)N1CCN(C(=O)c2ccc(O[C@@H]3N(C(=O)N[C@H](CC)c4ccc5c(c4)OCO5)C(=O)C3(CC)CC)cc2)CC1]

In some embodiments described above or below of a compound of Formula (I), Y¹ and Y² are the same. In some embodiments described above or below of a compound of Formula (I), Y¹ and Y² are both

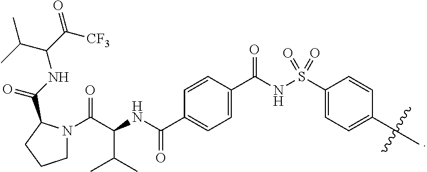 [SMILES: *c1ccc(S(=O)(=O)NC(=O)c2ccc(C(=O)N[C@H](C(=O)N3CCC[C@H]3C(=O)NC(C(=O)C(F)(F)F)C(C)C)C(C)C)cc2)cc1]

In some embodiments described above or below of a compound of Formula (I), Y¹ and Y² are both

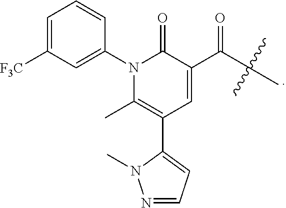 [SMILES: *C(=O)c1cc(-c2ccnn2C)c(C)n(-c2cccc(C(F)(F)F)c2)c1=O]

In some embodiments described above or below of a compound of Formula (I), Y¹ and Y² are both

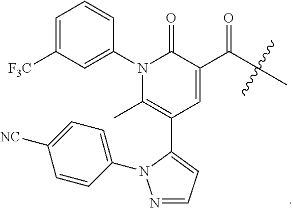 [SMILES: *C(=O)c1cc(-c2ccnn2-c2ccc(C#N)cc2)c(C)n(-c2cccc(C(F)(F)F)c2)c1=O]

In some embodiments described above or below of a compound of Formula (I), Y¹ and Y² are both

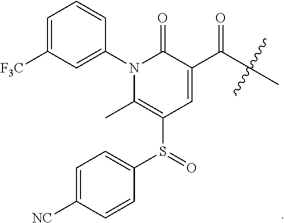 [SMILES: *C(=O)c1cc(S(=O)c2ccc(C#N)cc2)c(C)n(-c2cccc(C(F)(F)F)c2)c1=O]

In some embodiments described above or below of a compound of Formula (I), Y¹ and Y² are both

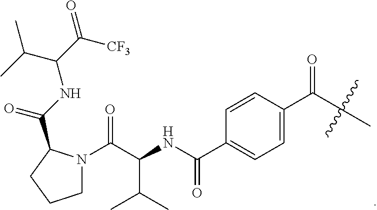 [SMILES: *C(=O)c1ccc(C(=O)N[C@H](C(=O)N2CCC[C@H]2C(=O)NC(C(=O)C(F)(F)F)C(C)C)C(C)C)cc1]

In some embodiments described above or below of a compound of Formula (I), Y¹ and Y² are both

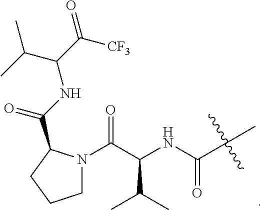 [SMILES: *C(=O)N[C@H](C(=O)N1CCC[C@H]1C(=O)NC(C(=O)C(F)(F)F)C(C)C)C(C)C]

In some embodiments described above or below of a compound of Formula (I), Y¹ and Y² are both

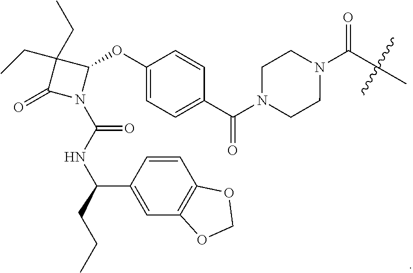 [SMILES: *C(=O)N1CCN(C(=O)c2ccc(O[C@@H]3N(C(=O)N[C@H](CCC)c4ccc5c(c4)OCO5)C(=O)C3(CC)CC)cc2)CC1]

In some embodiments described above or below of a compound of Formula (I), A¹ and A² are the same. In some embodiments described above or below of a compound of Formula (I), A¹ and A² are not the same. In some embodiments described above or below of a compound of Formula (I), A¹ and A² are both

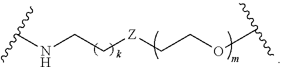

In some embodiments described above or below of a compound of Formula (I), A¹ and A² are both

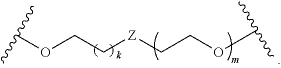

In some embodiments described above or below of a compound of Formula (I), A¹ and A² are both

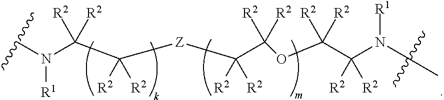

In some embodiments described above or below of a compound of Formula (I), Z is O.

In some embodiments described above or below of a compound of Formula (I), k is 1.

In some embodiments described above or below of a compound of Formula (I), each m is independently 0, 1, 2, or 3.

In some embodiments described above or below of a compound of Formula (I), X is

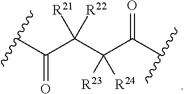

In some embodiments described above or below of a compound of Formula (I), X is

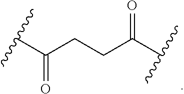 [SMILES: *C(=O)CCC(*)=O]

In some embodiments described above or below of a compound of Formula (I), R²¹ and R²³ are the same and R²² and R²⁴ are the same. In some embodiments described above or below of a compound of Formula (I), X is

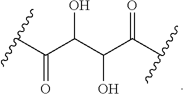 [SMILES: *C(=O)C(O)C(O)C(*)=O]

In some embodiments described above or below of a compound of Formula (I), X is

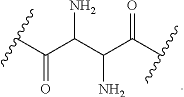 [SMILES: *C(=O)C(N)C(N)C(*)=O]

In some embodiments described above or below of a compound of Formula (I), R²¹ and R²² are the same. In some embodiments described above or below of a compound of Formula (I), R²³ and R²⁴ are the same. In some embodiments described above or below of a compound of Formula (I), R²¹, R²², R²³, and R²⁴ are the same.

In some embodiments described above or below of a compound of Formula (I), X is

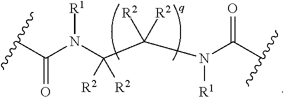

In certain embodiments described above or below of a compound of Formula (I), X is

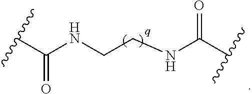

In some embodiments described above or below of a compound of Formula (I), q is 3. In some embodiments described above or below of a compound of Formula (I), q is 5.

In some embodiments described above or below of a compound of Formula (I), X is

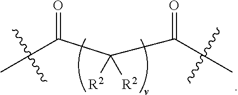

In certain embodiments described above or below of a compound of Formula (I), X is

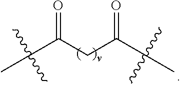

In certain embodiments described above or below of a compound of Formula (I), X is

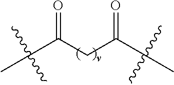

and v is 4.

In some embodiments described above or below of a compound of Formula (I), X is

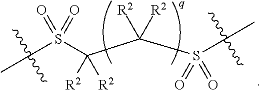

In certain embodiments described above or below of a compound of Formula (I), X is

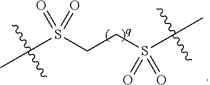

In certain embodiments described above or below of a compound of Formula (I), X is

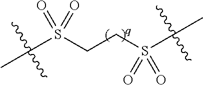

and q is 3.

In some embodiments described above or below of a compound of Formula (I), X is

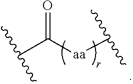

In certain embodiments described above or below of a compound of Formula (I), aa is selected from:

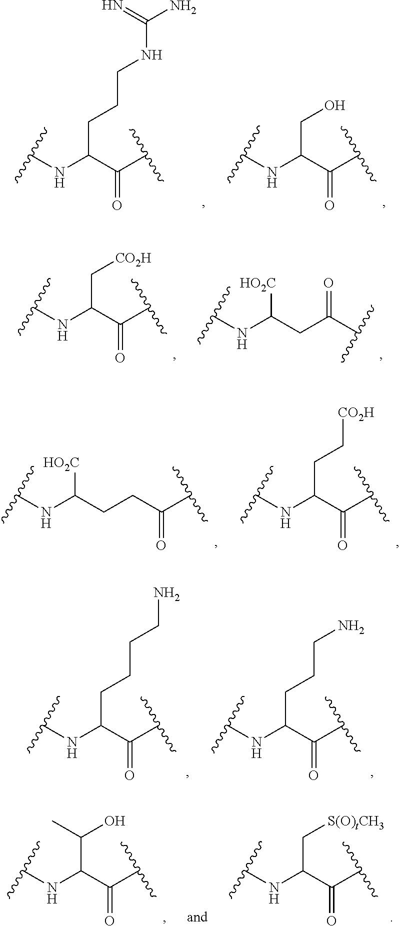

In some embodiments described above or below of a compound of Formula (I), r is 4, 5, or 6.

In some embodiments described above or below of a compound of Formula (I), X is

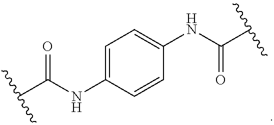 [SMILES: *C(=O)Nc1ccc(NC(*)=O)cc1]

In some embodiments described above or below of a compound of Formula (I), X is

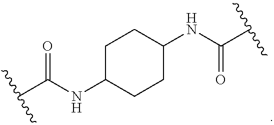 [SMILES: *C(=O)NC1CCC(NC(*)=O)CC1]

In some embodiments described above or below of a compound of Formula (I), X is alkylene. In some embodiments described above or below of a compound of Formula (I), X is —CH₂CH₂—. In some embodiments described above or below of a compound of Formula (I), X is —CH₂CH₂CH₂—.

In some embodiments described above or below of a compound of Formula (I), X is —C(O)—. In some embodiments described above or below of a compound of Formula (I), X is —C(O)C(O)—.

Also provided herein is a pharmaceutical composition comprising a compound of Formula (I) or as described above and below, or a pharmaceutically acceptable salt, solvate, polymorph, prodrug, metabolite, N-oxide, stereoisomer, or isomer thereof, and a pharmaceutically acceptable excipient.

Further provided herein is a method of treating a lung disease, the method comprising administering a compound of Formula (I), or a pharmaceutically acceptable salt, solvate, polymorph, prodrug, metabolite, deuteride, N-oxide, stereoisomer, or isomer thereof, as described above and below.

Further provided herein is a method to treat chronic obstructive pulmonary disease (COPD) or cystic fibrosis, the method comprising administering a compound of Formula (I), or a pharmaceutically acceptable salt, solvate, polymorph, prodrug, metabolite, deuteride, N-oxide, stereoisomer, or isomer thereof, as described above and below.

Further provided herein is a method of treating an intestinal disease, the method comprising administering a compound of Formula (I), or a pharmaceutically acceptable salt, solvate, polymorph, prodrug, metabolite, deuteride, N-oxide, stereoisomer, or isomer thereof, as described above and below.

Further provided herein is a method to treat inflammatory bowel disease (IBD), the method comprising administering a compound of Formula (I), or a pharmaceutically acceptable salt, solvate, polymorph, prodrug, metabolite, deuteride, N-oxide, stereoisomer, or isomer thereof, as described above and below.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

Human neutrophil elastase released extracellularly at sites of inflammation plays an important role in the pathogenesis of a range of inflammatory diseases including COPD, cystic fibrosis, and IBD.

In COPD patients, damage to lung connective tissue from smoke induced inflammation results from the massive migration of neutrophils to the lungs and the subsequent release of proteolytic enzymes. Inadequate control of the activity of these enzymes due to depressed levels of their physiological protein inhibitors leads to a protease/antiprotease imbalance in the airways, ultimately allowing the degradation of elastin, the elastic component of lung connective tissue. Therefore, elastase inhibitors can serve to protect the lungs by blocking the breakdown of elastin in the lung connective tissue.

In IBD patients, fecal neutrophil elastase levels are increased and show correlation with disease activity. The infiltration of inflammatory leukocytes, including polymorphonuclear neutrophils, is a characteristic histological feature of mucosal lesions in IBD. Consequently, inhibition of neutrophil elastase activity in the inflamed bowel with an elastase inhibitor can reduce gastrointestinal tissue damage in IBD patients.

Disclosed herein are dimeric compounds useful for the treatment of lung diseases including COPD and cystic fibrosis. Also disclosed herein are dimeric compounds useful for the treatment of intestinal diseases including IBD. The compounds disclosed herein are elastase inhibitors that demonstrate improved potency and/or PK properties. Also disclosed herein are compositions comprising such compounds, and methods of their use for the inhibition of elastase and the treatment of lung diseases including COPD and cystic fibrosis. Also disclosed herein are compositions comprising such compounds, and methods of their use for the inhibition of elastase and the treatment of intestinal diseases including IBD.

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a”, “an”, and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“Amino” refers to the —NH₂ radical.

“Cyano” or “nitrile” refers to the —CN radical.

“Hydroxy” or “hydroxyl” refers to the —OH radical.

“Nitro” refers to the —NO₂ radical.

“Oxo” refers to the ═O substituent.

“Oxime” refers to the ═N—OH substituent.

“Thioxo” refers to the ═S substituent.

“Alkyl” refers to a straight or branched hydrocarbon chain radical, has from one to thirty carbon atoms, and is attached to the rest of the molecule by a single bond. Alkyls comprising any number of carbon atoms from 1 to 30 are included. An alkyl comprising up to 30 carbon atoms is referred to as a C₁-C₃₀ alkyl, likewise, for example, an alkyl comprising up to 12 carbon atoms is a C₁-C₁₂ alkyl. Alkyls (and other moieties defined herein) comprising other numbers of carbon atoms are represented similarly. Alkyl groups include, but are not limited to, C₁-C₃₀ alkyl, C₁-C₂₀ alkyl, C₁-C₁₅ alkyl, C₁-C₁₀ alkyl, C₁-C₈ alkyl, C₁-C₆ alkyl, C₁-C₄ alkyl, C₁-C₃ alkyl, C₁-C₂ alkyl, C₂-C₈ alkyl, C₃-C₈ alkyl and C₄-C₈ alkyl. Representative alkyl groups include, but are not limited to, methyl, ethyl, n-propyl, 1-methylethyl (iso-propyl), n-butyl, i-butyl, s-butyl, n-pentyl, 1,1-dimethylethyl (t-butyl), 3-methylhexyl, 2-methylhexyl, vinyl, allyl, propynyl, and the like. Alkyl comprising unsaturations include alkenyl and alkynyl groups. Unless stated otherwise specifically in the specification, an alkyl group may be optionally substituted as described below.

“Alkylene” or “alkylene chain” refers to a straight or branched divalent hydrocarbon chain, as described for alkyl above. Unless stated otherwise specifically in the specification, an alkylene group may be optionally substituted as described below.

“Alkoxy” refers to a radical of the formula —OR, where Rₐ is an alkyl radical as defined. Unless stated otherwise specifically in the specification, an alkoxy group may be optionally substituted as described below.

“Aryl” refers to a radical derived from a hydrocarbon ring system comprising hydrogen, 6 to 30 carbon atoms and at least one aromatic ring. The aryl radical may be a monocyclic, bicyclic, tricyclic or tetracyclic ring system, which may include fused or bridged ring systems. Aryl radicals include, but are not limited to, aryl radicals derived from the hydrocarbon ring systems of aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” or the prefix “ar-” (such as in “aralkyl”) is meant to include aryl radicals that are optionally substituted.

“Cycloalkyl” or “carbocycle” refers to a stable, non-aromatic, monocyclic or polycyclic carbocyclic ring, which may include fused or bridged ring systems, which is saturated or unsaturated. Representative cycloalkyls or carbocycles include, but are not limited to, cycloalkyls having from three to fifteen carbon atoms, from three to ten carbon atoms, from three to eight carbon atoms, from three to six carbon atoms, from three to five carbon atoms, or three to four carbon atoms. Monocyclic cycloalkyls or carbocycles include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Unless otherwise stated specifically in the specification, a cycloalkyl or carbocycle group may be optionally substituted. Illustrative examples of cycloalkyl groups include, but are not limited to, the following moieties:

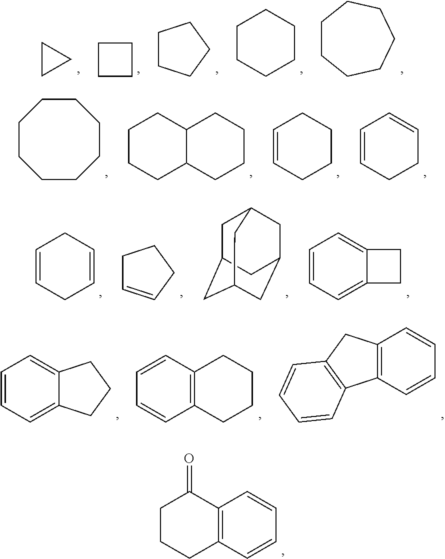

and the like.

“Fused” refers to any ring structure described herein which is fused to an existing ring structure. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” or “halogen” refers to bromo, chloro, fluoro or iodo.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

“Haloalkoxy” similarly refers to a radical of the formula —OR, where Rₐ is a haloalkyl radical as defined. Unless stated otherwise specifically in the specification, a haloalkoxy group may be optionally substituted as described below.

“Heterocycloalkyl” or “heterocyclyl” or “heterocyclic ring” or “heterocycle” refers to a stable 3- to 24-membered non-aromatic ring radical comprising 2 to 23 carbon atoms and from one to 8 heteroatoms selected from the group consisting of nitrogen, oxygen, phosphorus and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 12-crown-4, 15-crown-5, 18-crown-6, 21-crown-7, aza-18-crown-6, diaza-18-crown-6, aza-21-crown-7, and diaza-21-crown-7. Unless stated otherwise specifically in the specification, a heterocyclyl group may be optionally substituted. Illustrative examples of heterocycloalkyl groups, also referred to as non-aromatic heterocycles, include:

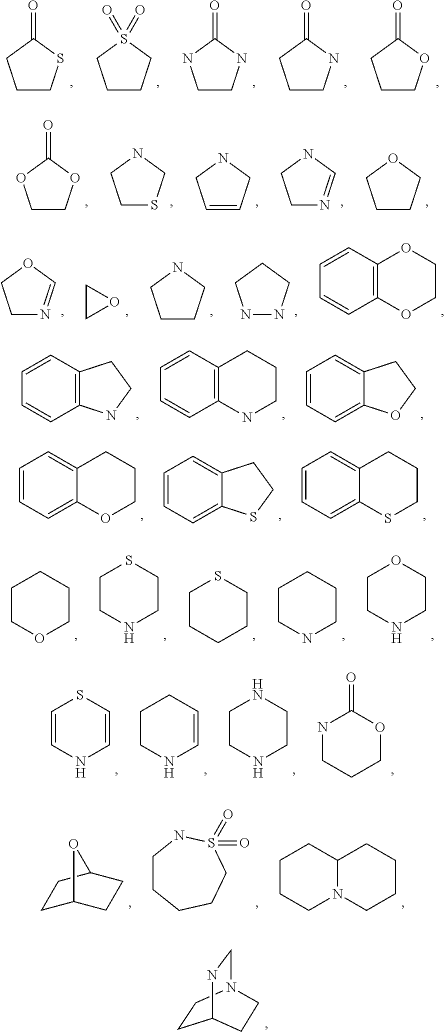

and the like. The term heterocycloalkyl also includes all ring forms of the carbohydrates, including but not limited to the monosaccharides, the disaccharides and the oligosaccharides. Unless otherwise noted, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl group may be optionally substituted.

The term “heteroaryl” as used herein, alone or in combination, refers to optionally substituted aromatic monoradicals containing from about five to about twenty skeletal ring atoms, where one or more of the ring atoms is a heteroatom independently selected from among oxygen, nitrogen, sulfur, phosphorus, silicon, selenium and tin but not limited to these atoms and with the proviso that the ring of said group does not contain two adjacent O or S atoms. In embodiments in which two or more heteroatoms are present in the ring, the two or more heteroatoms can be the same as each another, or some or all of the two or more heteroatoms can each be different from the others. The term heteroaryl includes optionally substituted fused and non-fused heteroaryl radicals having at least one heteroatom. The term heteroaryl also includes fused and non-fused heteroaryls having from five to about twelve skeletal ring atoms, as well as those having from five to about ten skeletal ring atoms. Bonding to a heteroaryl group can be via a carbon atom or a heteroatom. Thus, as a non-limiting example, an imidiazole group may be attached to a parent molecule via any of its carbon atoms (imidazol-2-yl, imidazol-4-yl or imidazol-5-yl), or its nitrogen atoms (imidazol-1-yl or imidazol-3-yl). Likewise, a heteroaryl group may be further substituted via any or all of its carbon atoms, and/or any or all of its heteroatoms. A fused heteroaryl radical may contain from two to four fused rings where the ring of attachment is a heteroaromatic ring and the other individual rings may be alicyclic, heterocyclic, aromatic, heteroaromatic or any combination thereof. A non-limiting example of a single ring heteroaryl group includes pyridyl; fused ring heteroaryl groups include benzimidazolyl, quinolinyl, acridinyl; and a non-fused bi-heteroaryl group includes bipyridinyl. Further examples of heteroaryls include, without limitation, furanyl, thienyl, oxazolyl, acridinyl, azepinyl, phenazinyl, benzimidazolyl, benzindolyl,benzofuranyl, benzofuranonyl,benzoxazolyl, benzothiazolyl, benzothiadiazolyl, benzothiophenyl, benzoxadiazolyl, benzodioxolyl, benzo[b][1,4]dioxepinyl, 1,4-benzodioxanyl, benzonaphthofuranyl, benzotriazolyl, benzodioxolyl, benzodioxinyl, benzopyranyl, benzopyranonyl, benzothienyl (benzothiophenyl), benzo[4,6]imidazo[1,2-a]pyridinyl, carbazolyl, cinnolinyl, dibenzofuranyl, dibenzothiophenyl, furanonyl, imidazolyl, indolyl, isoxazolyl, isoquinolinyl, indolizinyl, indazolyl, isoindolyl, indolinyl, isoindolinyl, indolizinyl, isothiazolyl, isoindolyloxadiazolyl, indazolyl, naphthyridinyl, oxadiazolyl, 2-oxoazepinyl, oxiranyl, 1-oxidopyridinyl, 1-oxidopyrimidinyl, 1-oxidopyrazinyl, 1-oxidopyridazinyl, 1-phenyl-1H-pyrrolyl, phenothiazinyl, phenoxazinyl, pyridinyl, pyridazinyl, pyrimidinyl, pyrazinyl, pyrrolyl, pyrazinyl, pyrazolyl, purinyl, phthalazinyl, pteridinyl, quinolinyl, quinazolinyl, quinoxalinyl, quinuclidinyl, triazolyl, tetrazolyl, thiazolyl, triazinyl, thiadiazolyl, tetrahydroquinolinyl, thiazolyl, and thiophenyl and the like, and their oxides, such as for example pyridyl-N-oxide. Illustrative examples of heteroaryl groups include the following moieties:

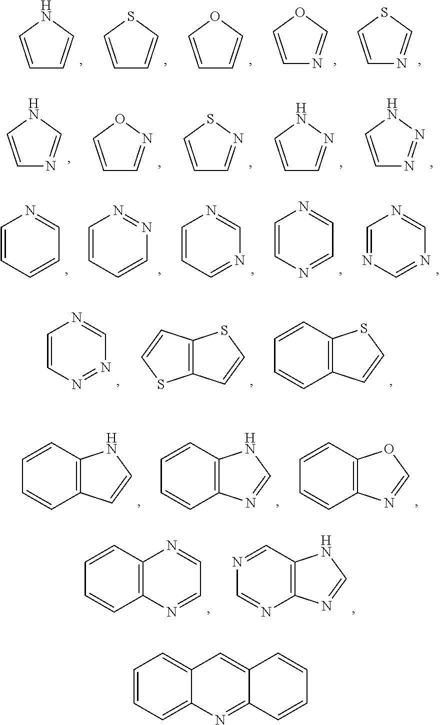

and the like.

All the above groups may be either substituted or unsubstituted. The term “substituted” as used herein means any of the above groups (e.g, alkyl, alkylene, alkoxy, aryl, cycloalkyl, haloalkyl, heterocyclyl and/or heteroaryl) may be further functionalized wherein at least one hydrogen atom is replaced by a bond to a non-hydrogen atom substituent. Unless stated specifically in the specification, a substituted group may include one or more substituents selected from: oxo, amino, —CO₂H, nitrile, nitro, hydroxyl, thiooxy, alkyl, alkylene, alkoxy, aryl, cycloalkyl, heterocyclyl, heteroaryl, dialkylamines, arylamines, alkylarylamines, diarylamines, trialkylammonium (—N⁺R₃), N-oxides, imides, and enamines; a silicon atom in groups such as trialkylsilyl groups, dialkylarylsilyl groups, alkyldiarylsilyl groups, triarylsilyl groups, perfluoroalkyl or perfluoroalkoxy, for example, trifluoromethyl or trifluoromethoxy. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NH₂, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. In the foregoing, R_{g} and Rₕ are the same or different and independently hydrogen, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl. In addition, each of the foregoing substituents may also be optionally substituted with one or more of the above substituents. Furthermore, any of the above groups may be substituted to include one or more internal oxygen, sulfur, or nitrogen atoms. For example, an alkyl group may be substituted with one or more internal oxygen atoms to form an ether or polyether group. Similarly, an alkyl group may be substituted with one or more internal sulfur atoms to form a thioether, disulfide, etc.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, any substituents described should generally be understood as having a maximum molecular weight of about 1,000 daltons, and more typically, up to about 500 daltons.

The label

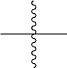

in a compound fragment denotes the attachment point to the remainder of the compound. Compound fragments containing two

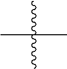

can be attached to the remainder of the compound in either orientation. For example, when A¹ is

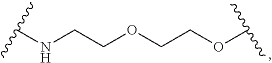 [SMILES: *NCCOCCO*]

the compound of Formula (I) can be

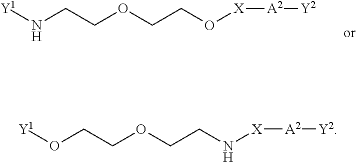

An “effective amount” or “therapeutically effective amount” refers to an amount of a compound administered to a mammalian subject, either as a single dose or as part of a series of doses, which is effective to produce a desired therapeutic effect.

“Treatment” of an individual (e.g. a mammal, such as a human) or a cell is any type of intervention used in an attempt to alter the natural course of the individual or cell. In some embodiments, treatment includes administration of a pharmaceutical composition, subsequent to the initiation of a pathologic event or contact with an etiologic agent and includes stabilization of the condition (e.g., condition does not worsen) or alleviation of the condition. In other embodiments, treatment also includes prophylactic treatment (e.g., administration of a composition described herein when an individual is suspected to be suffering from a bacterial infection).

A “tautomer” refers to a proton shift from one atom of a molecule to another atom of the same molecule. The compounds presented herein may exist as tautomers. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH. Some examples of tautomeric interconversions include:

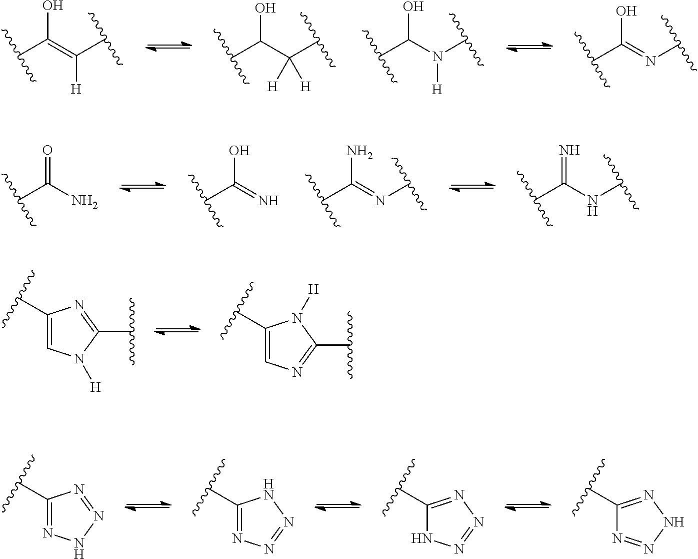

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes, such as, oxidation reactions) by which a particular substance is changed by an organism. Thus, enzymes may produce specific structural alterations to a compound. For example, cytochrome P450 catalyzes a variety of oxidative and reductive reactions while uridine diphosphate glucuronyl transferases catalyze the transfer of an activated glucuronic-acid molecule to aromatic alcohols, aliphatic alcohols, carboxylic acids, amines and free sulfhydryl groups. Further information on metabolism may be obtained from The Pharmacological Basis of Therapeutics, 9th Edition, McGraw-Hill (1996). Metabolites of the compounds disclosed herein can be identified either by administration of compounds to a host and analysis of tissue samples from the host, or by incubation of compounds with hepatic cells in vitro and analysis of the resulting compounds. Both methods are well known in the art. In some embodiments, metabolites of a compound are formed by oxidative processes and correspond to the corresponding hydroxy-containing compound. In some embodiments, a compound is metabolized to pharmacologically active metabolites.

### Compounds

Described herein are compounds for the treatment of a lung disease. Further described herein are compounds for the treatment of COPD or cystic fibrosis.

Also described herein are compounds for the treatment of an intestinal disease. Further described herein are compounds for the treatment of IBD.

In one aspect, provided herein are compounds of Formula (I), Y¹-A¹-X-A²-Y², or pharmaceutically acceptable salts, solvates, polymorphs, prodrugs, metabolites, deuterides, N-oxides, stereoisomers, or isomers thereof, wherein Y¹ and Y² are derived from elastase inhibitors.

In a certain aspect, provided herein are compounds of Formula (I), or pharmaceutically acceptable salts, solvates, polymorphs, prodrugs, metabolites, deuterides, N-oxides, stereoisomers, or isomers thereof:

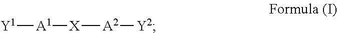

wherein:
A¹ and A² are independently selected from

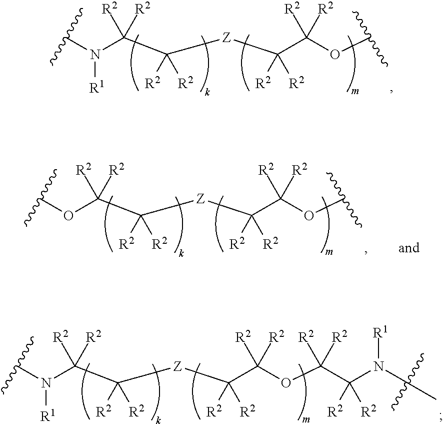

Y¹ and Y² are independently an elastase inhibitor;
X is alkylene, —C(O)—, —C(O)C(O)—,

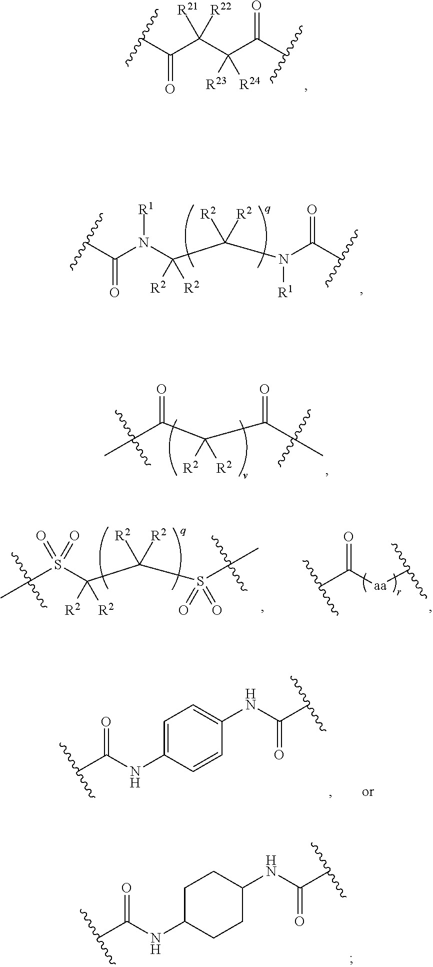

Z is O or CR²R²;
aa is

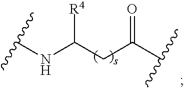

each R¹ is independently selected from H, alkyl, haloalkyl, arylalkyl, and heteroarylalkyl;
each R² is independently selected from H, halo, —CN, —SR¹, alkyl, cycloalkyl, haloalkyl, —OR¹, and —R⁴;
each R⁴ is independently selected from —CO₂R¹, -(alkylene)-(CO₂R¹), hydroxyalkyl, -(alkylene)(S(O)ₜ)(alkyl), -(alkylene)(NR⁵R⁵), and

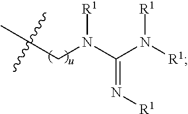

each R⁵ is independently selected from H, alkyl, haloalkyl, arylalkyl, heteroarylalkyl, aryl, and heteroaryl;
R²¹, R²², R²³, and R²⁴ are independently selected from H, halo, —CN, —SR¹, alkyl, cycloalkyl, haloalkyl, —NR¹R¹, and —OR¹;
each k is independently selected from 1, 2, 3, 4, 5, 6, 7, 8, 9, and 10;
each m is independently selected from 0, 1, 2, 3, 4, 5, 6, 7, 8, 9, and 10;
q is 1, 2, 3, 4, 5, 6, 7, 8, 9, or 10;
r is 3, 4, 5, 6, or 7;
each s is independently selected from 0, 1, 2, 3, and 4;
each t is independently selected from 0, 1, and 2;
each u is independently selected from 1, 2, 3, 4, 5, 6, 7, 8, 9, and 10; and
v is selected from 3, 4, 5, 6, 7, 8, 9, and 10.

In some embodiments, provided herein are compounds of Formula (Ia), or pharmaceutically acceptable salts, solvates, polymorphs, prodrugs, metabolites, deuterides, N-oxides, stereoisomers, or isomers thereof:

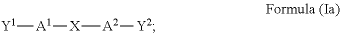

wherein:
A¹ and A² are independently selected from:

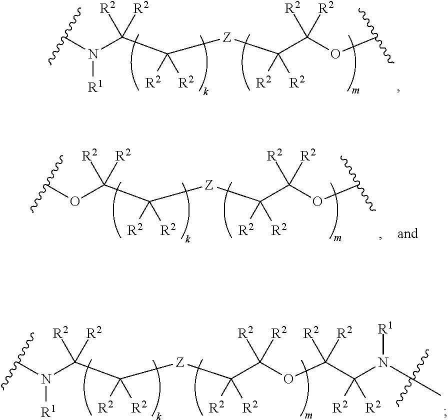

Y¹ and Y² are independently selected from

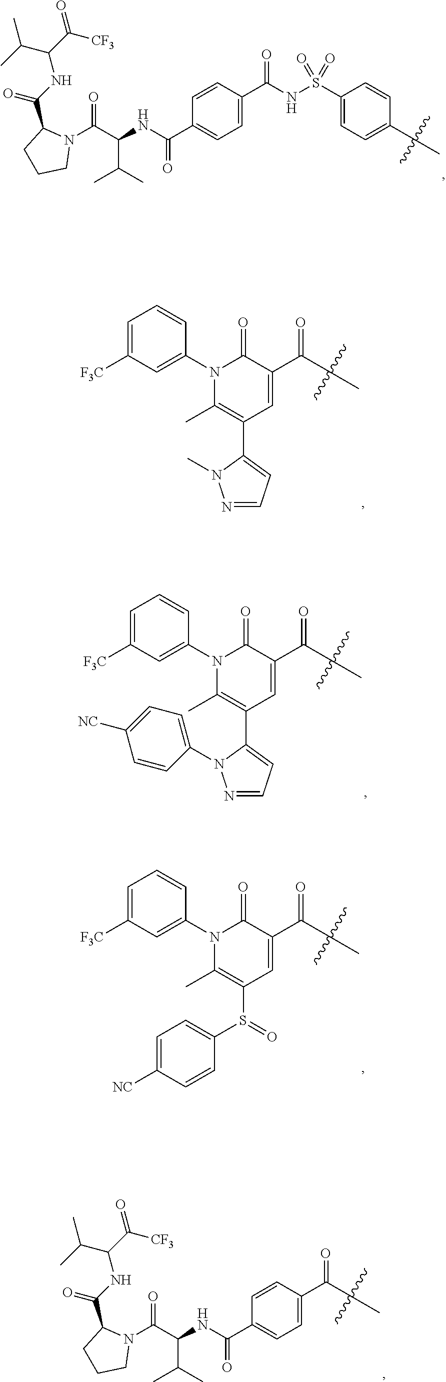 [SMILES: *c1ccc(S(=O)(=O)NC(=O)c2ccc(C(=O)N[C@H](C(=O)N3CCC[C@H]3C(=O)NC(C(=O)C(F)(F)F)C(C)C)C(C)C)cc2)cc1 ; *C(=O)c1cc(-c2ccnn2C)c(C)n(-c2cccc(C(F)(F)F)c2)c1=O ; *C(=O)c1cc(-c2ccnn2-c2ccc(C#N)cc2)c(C)n(-c2cccc(C(F)(F)F)c2)c1=O ; *C(=O)c1cc(S(=O)c2ccc(C#N)cc2)c(C)n(-c2cccc(C(F)(F)F)c2)c1=O ; *C(=O)c1ccc(C(=O)N[C@H](C(=O)N2CCC[C@H]2C(=O)NC(C(=O)C(F)(F)F)C(C)C)C(C)C)cc1]

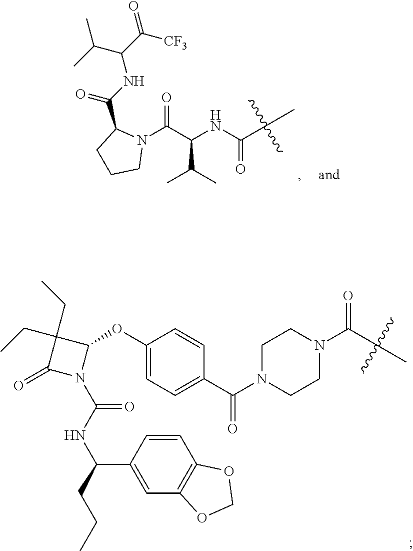 [SMILES: *C(=O)N[C@H](C(=O)N1CCC[C@H]1C(=O)NC(C(=O)C(F)(F)F)C(C)C)C(C)C ; *C(=O)N1CCN(C(=O)c2ccc(O[C@@H]3N(C(=O)N[C@H](CCC)c4ccc5c(c4)OCO5)C(=O)C3(CC)CC)cc2)CC1]

X is alkylene, —C(O)—, —C(O)C(O)—,

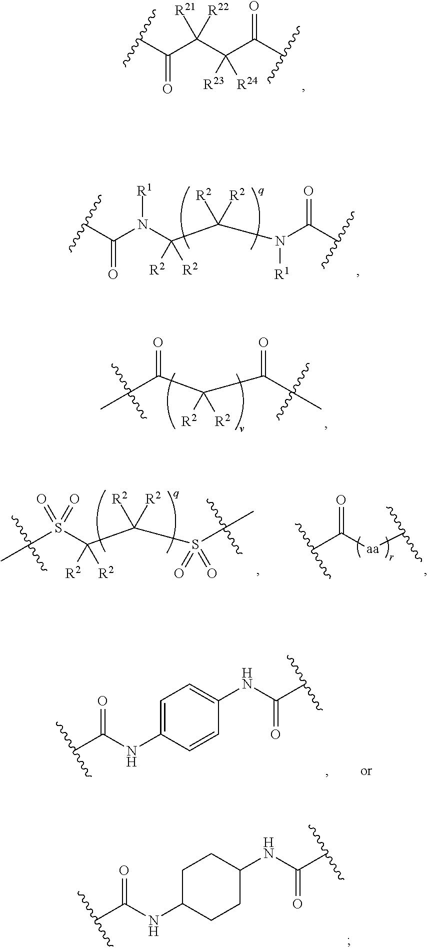

Z is O or CR²R²;
aa is

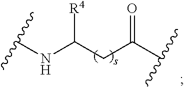

each R¹ is independently selected from H, alkyl, haloalkyl, arylalkyl, and heteroarylalkyl;
each R² is independently selected from H, halo, —CN, —SR¹, alkyl, cycloalkyl, haloalkyl, —OR¹, and —R⁴;
each R⁴ is independently selected from —CO₂R¹, -(alkylene)-(CO₂R¹), hydroxyalkyl, -(alkylene)(S(O)ₜ)(alkyl), -(alkylene)(NR⁵R⁵), and

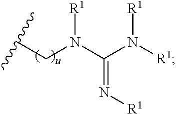

each R⁵ is independently selected from H, alkyl, haloalkyl, arylalkyl, heteroarylalkyl, aryl, and heteroaryl;
R²¹, R²², R²³, and R²⁴ are independently selected from H, halo, —CN, —SR¹, alkyl, cycloalkyl, haloalkyl, —NR¹R¹, and —OR¹;
each k is independently selected from 1, 2, 3, 4, 5, 6, 7, 8, 9, and 10;
each m is independently selected from 0, 1, 2, 3, 4, 5, 6, 7, 8, 9, and 10;
q is 1, 2, 3, 4, 5, 6, 7, 8, 9, or 10;
r is 3, 4, 5, 6, or 7;
each s is independently selected from 0, 1, 2, 3, and 4;
each t is independently selected from 0, 1, and 2;
each u is independently selected from 1, 2, 3, 4, 5, 6, 7, 8, 9, and 10; and
v is selected from 3, 4, 5, 6, 7, 8, 9, and 10.

In some embodiments described above or below of a compound of Formula (I) or (Ia), Y¹ and Y² are the same. In some embodiments described above or below of a compound of Formula (I) or (Ia), Y¹ and Y² are

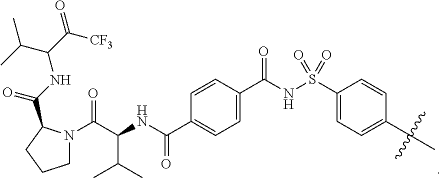 [SMILES: *c1ccc(S(=O)(=O)NC(=O)c2ccc(C(=O)N[C@H](C(=O)N3CCC[C@H]3C(=O)NC(C(=O)C(F)(F)F)C(C)C)C(C)C)cc2)cc1]

In some embodiments described above or below of a compound of Formula (I) or (Ia), Y¹ and Y² are

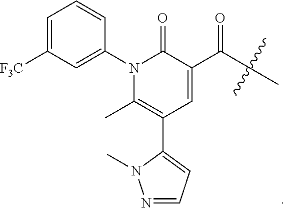 [SMILES: *C(=O)c1cc(-c2ccnn2C)c(C)n(-c2cccc(C(F)(F)F)c2)c1=O]

In some embodiments described above or below of a compound of Formula (I) or (Ia), A¹ and A² are the same. In some embodiments described above or below of a compound of Formula (I) or (Ia), A¹ and A² are both

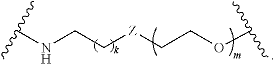

In some embodiments described above or below of a compound of Formula (I) or (Ia), A¹ and A² are both

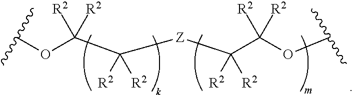

In some embodiments described above or below of a compound of Formula (I) or (Ia), A¹ and A² are both

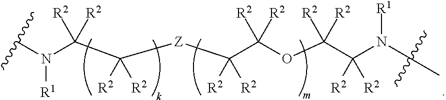

In some embodiments described above or below of a compound of Formula (I) or (Ia), Z is O. In certain embodiments described above or below of a compound of Formula (I) or (Ia), each k is independently 1, 2, or 3. In certain embodiments described above or below of a compound of Formula (I) or (Ia), each k is 1. In certain embodiments described above or below of a compound of Formula (I) or (Ia), each m is independently 0, 1, 2, or 3. In certain embodiments described above or below of a compound of Formula (I) or (Ia), each m is 0. In certain embodiments described above or below of a compound of Formula (I) or (Ia), each m is 1. In certain embodiments described above or below of a compound of Formula (I) or (Ia), each m is 2. In certain embodiments described above or below of a compound of Formula (I) or (Ia), each m is 3.

In some embodiments described above or below of a compound of Formula (I) or (Ia), Z is CR²R². In some embodiments described above or below of a compound of Formula (I) or (Ia), Z is CH₂.

In some embodiments described above or below of a compound of Formula (I) or (Ia), L¹ and L² are the same.

In some embodiments described above or below of a compound of Formula (I) or (Ia), L¹ and L² are both

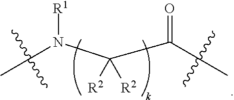

In some embodiments described above or below of a compound of Formula (I) or (Ia), L¹ and L² are both

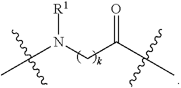

In some embodiments described above or below of a compound of Formula (I) or (Ia), L¹ and L² are both

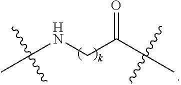

In some embodiments described above or below of a compound of Formula (I) or (Ia), L¹ and L² are both

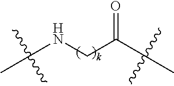

and k is 2. In some embodiments described above or below of a compound of Formula (I) or (Ia), L¹ and L² are both

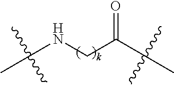

and k is 3. In some embodiments described above or below of a compound of Formula (I) or (Ia), L¹ and L² are both

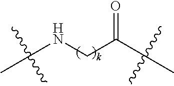

and k is 4. In some embodiments described above or below of a compound of Formula (I) or (Ia), L¹ and L² are both

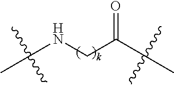

and k is 5.

In some embodiments described above or below of a compound of Formula (I) or (Ia), X is

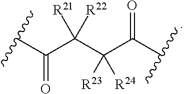

In some embodiments described above or below of a compound of Formula (I) or (Ia), R²¹ and R²³ are the same and R²² and R²⁴ are the same. In certain embodiments described above or below of a compound of Formula (I) or (Ia), X is

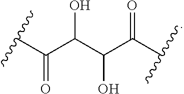 [SMILES: *C(=O)C(O)C(O)C(*)=O]

In certain embodiments described above or below of a compound of Formula (I) or (Ia), X is

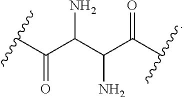 [SMILES: *C(=O)C(N)C(N)C(*)=O]

In some embodiments described above or below of a compound of Formula (I) or (Ia), R²¹ and R²² are the same. In some embodiments described above or below of a compound of Formula (I) or (Ia), R²³ and R²⁴ are the same. In some embodiments described above or below of a compound of Formula (I) or (Ia), R²¹, R²², R²³, and R²⁴ are the same.

In some embodiments described above or below of a compound of Formula (I) or (Ia), X is

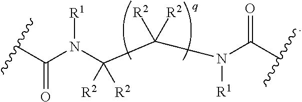

In some embodiments described above or below of a compound of Formula (I) or (Ia), X is

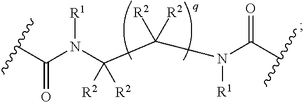

each R² is independently selected from H, alkyl, —OH, —CO₂H, —CO₂alkyl, and -(alkylene)(NH₂); and q is 1, 2, 3, 4, 5, or 6. In certain embodiments described above or below of a compound of Formula (I) or (Ia), X is

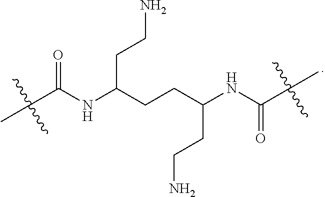 [SMILES: *C(=O)NC(CCN)CCC(CCN)NC(*)=O]

In certain embodiments described above or below of a compound of Formula (I) or (Ia), X is

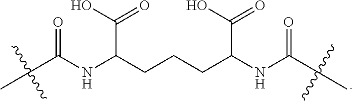 [SMILES: *C(=O)NC(CCCC(NC(*)=O)C(=O)O)C(=O)O]

In certain embodiments described above or below of a compound of Formula (I) or (Ia), X is

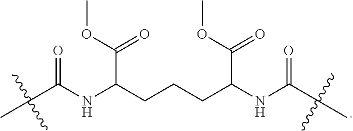 [SMILES: *C(=O)NC(CCCC(NC(*)=O)C(=O)OC)C(=O)OC]

In certain embodiments described above or below of a compound of Formula (I) or (Ia), X is

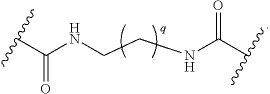

In certain embodiments described above or below of a compound of Formula (I) or (Ia), q is 1, 2, 3, 4, 5, or 6. In certain embodiments described above or below of a compound of Formula (I) or (Ia), q is 1. In certain embodiments described above or below of a compound of Formula (I) or (Ia), q is 2. In certain embodiments described above or below of a compound of Formula (I) or (Ia), q is 3. In certain embodiments described above or below of a compound of Formula (I) or (Ia), q is 4. In certain embodiments described above or below of a compound of Formula (I) or (Ia), q is 5. In certain embodiments described above or below of a compound of Formula (I) or (Ia), q is 6.

In some embodiments described above or below of a compound of Formula (I) or (Ia), X is

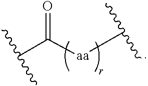

In certain embodiments described above or below of a compound of Formula (I) or (Ia), aa is selected from:

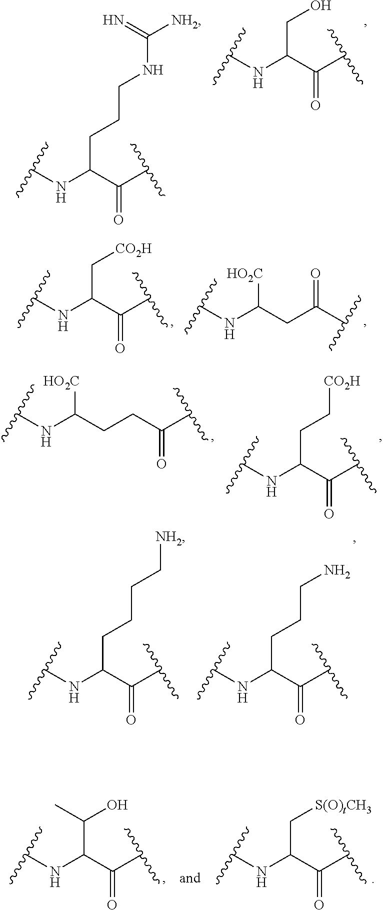

In some embodiments described above or below of a compound of Formula (I) or (Ia), r is 4, 5, or 6.

In some embodiments described above or below of a compound of Formula (I) or (Ia), X is

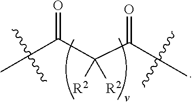

In some embodiments described above or below of a compound of Formula (I) or (Ia), X is

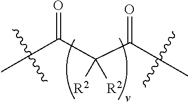

and each R² is independently selected from H, alkyl, —OH, —CO₂H, —CO₂alkyl, and -(alkylene)(NH₂). In certain embodiments described above or below of a compound of Formula (I) or (Ia), X is

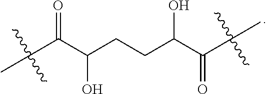 [SMILES: *C(=O)C(O)CCC(O)C(*)=O]

In certain embodiments described above or below of a compound of Formula (I) or (Ia), X is

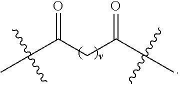

In certain embodiments described above or below of a compound of Formula (I) or (Ia), X is

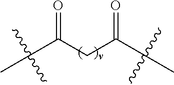

and v is 3. In certain embodiments described above or below of a compound of Formula (I) or (Ia), X is

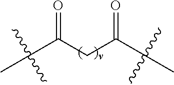

and v is 4. In certain embodiments described above or below of a compound of Formula (I) or (Ia), X is

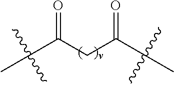

and v is 5. In certain embodiments described above or below of a compound of Formula (I) or (Ia), X is

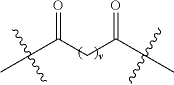

and v is 6.

In some embodiments described above or below of a compound of Formula (I) or (Ia), X is

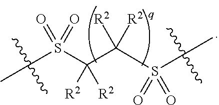

In certain embodiments described above or below of a compound of Formula (I) or (Ia), X is

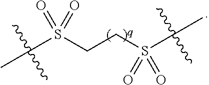

In certain embodiments described above or below of a compound of Formula (I) or (Ia), X is

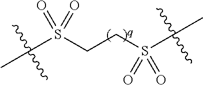

and q is 2. In certain embodiments described above or below of a compound of Formula (I) or (Ia), X is

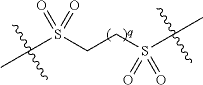

and q is 3. In certain embodiments described above or below of a compound of Formula (I) or (Ia), X is

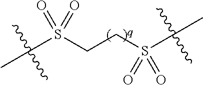

and q is 4.

In some embodiments described above or below of a compound of Formula (I) or (Ia), X is

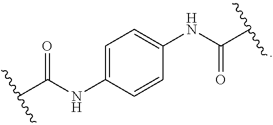 [SMILES: *C(=O)Nc1ccc(NC(*)=O)cc1]

In some embodiments described above or below of a compound of Formula (I) or (Ia), X is

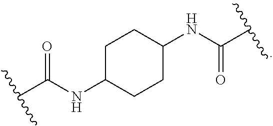 [SMILES: *C(=O)NC1CCC(NC(*)=O)CC1]

In some embodiments are compounds of Formula (I) or (Ia), wherein A¹-X-A² is a linker with the structure shown in Table 1 below and R is Y¹ and R′ is Y² wherein Y¹ and Y² are described above:

TABLE 1

| Linker | |
|---|---|
| # | A¹—X—A² |
| 1 | 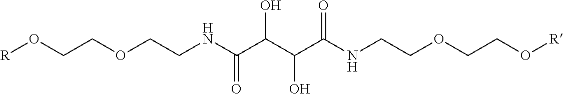 |
| 2 | 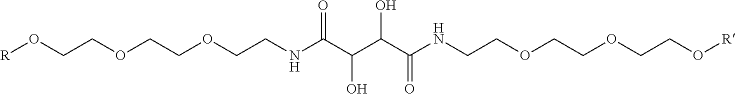 |
| 3 | 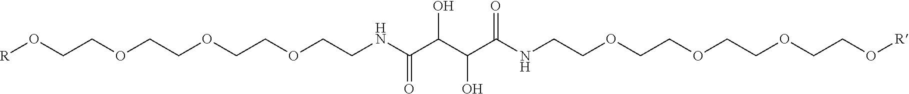 |
| 4 | 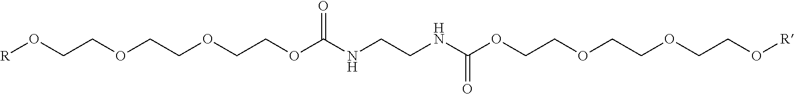 |
| 5 | 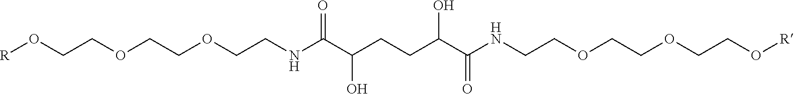 |
| 6 | 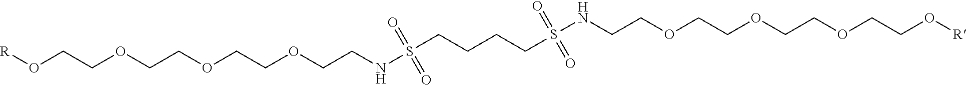 |
| 7 | 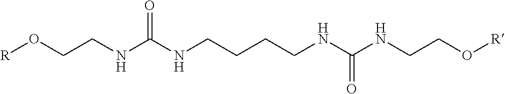 |
| 8 | 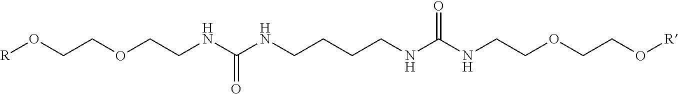 |
| 9 | 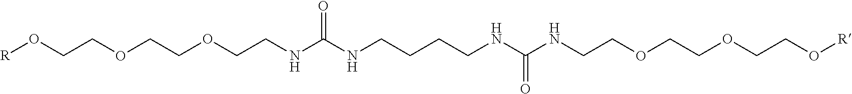 |
| 10 | 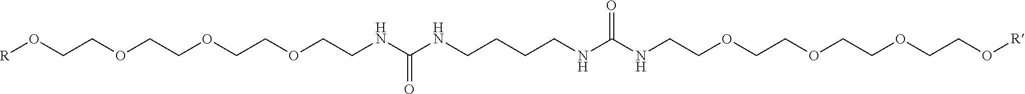 |
| 11 | 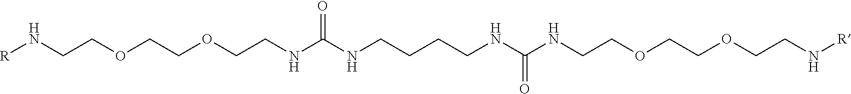 |
| 12 | 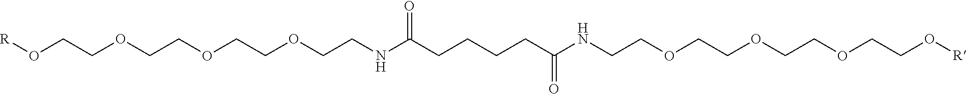 |
| 13 | 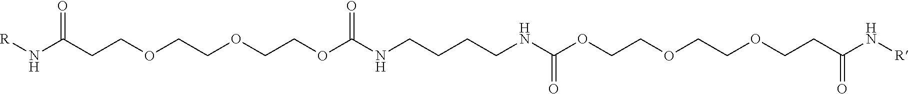 |
| 14 | 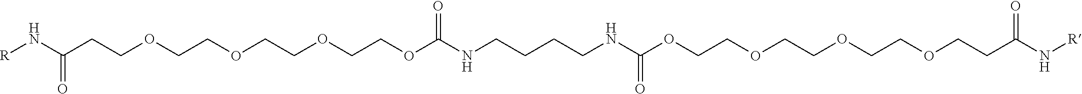 |
| 15 | 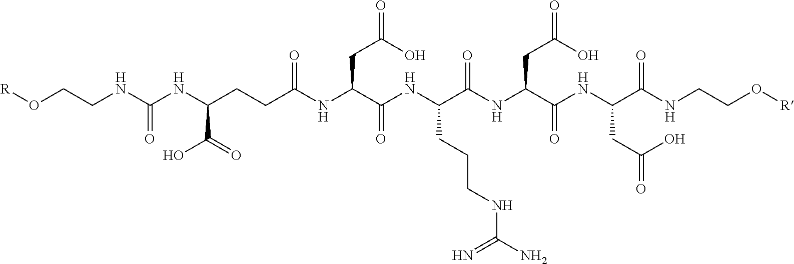 |
| 16 | 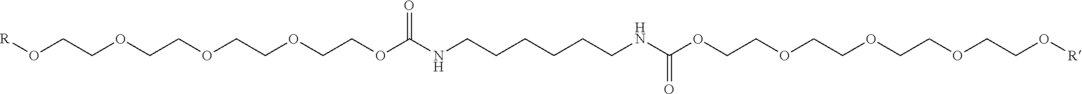 |
| 17 | 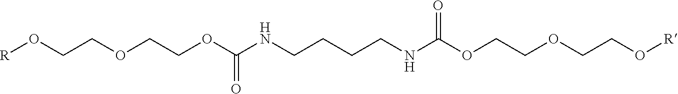 |
| 18 | 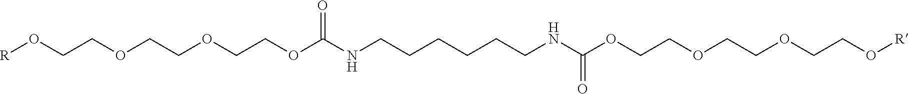 |
| 19 | 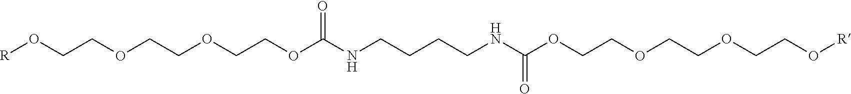 |
| 20 | 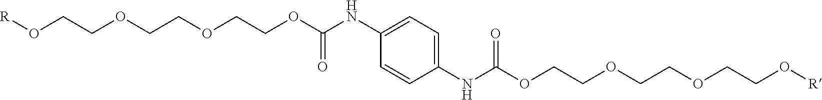 |
| 21 | 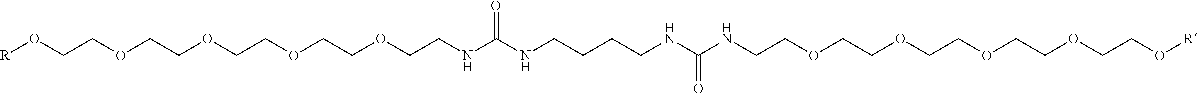 |
| 22 | 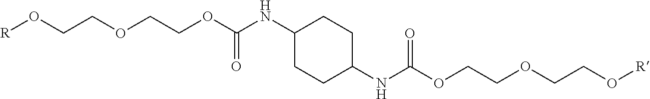 |
| 23 | 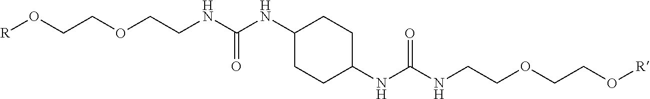 |
| 24 | 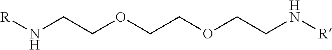 |
| 25 | 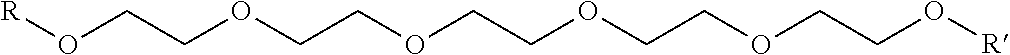 |
| 26 | 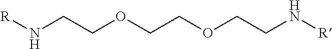 |
| 27 | 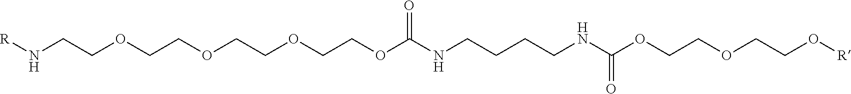 |
| 28 | 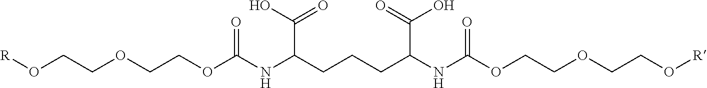 |
| 29 | 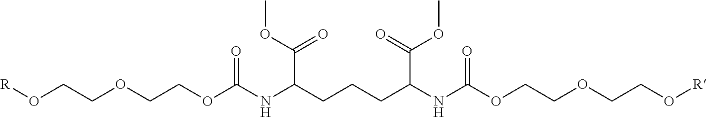 |
| 30 | 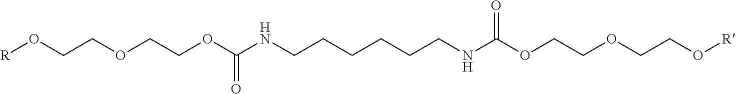 |
| 32 | 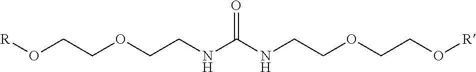 |
| 33 | 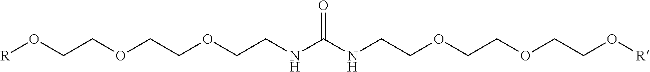 |
| 34 | 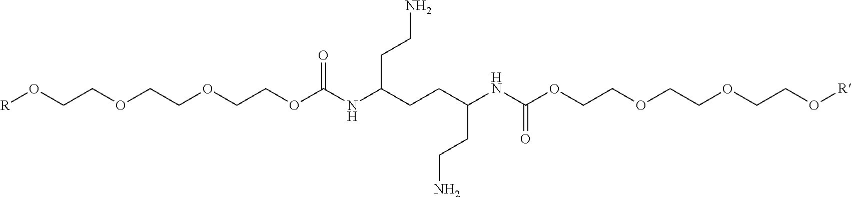 |
| 35 | 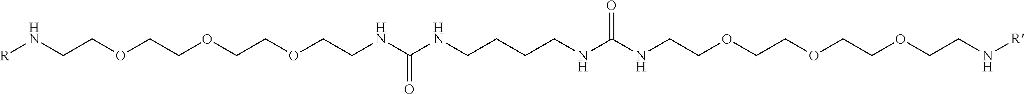 |
| 36 | 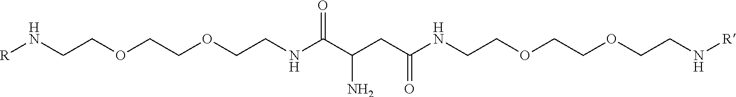 |
| 37 | 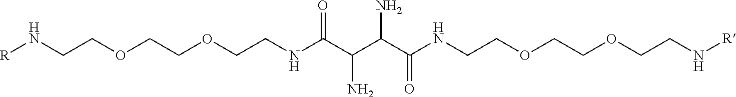 |
| 38 | 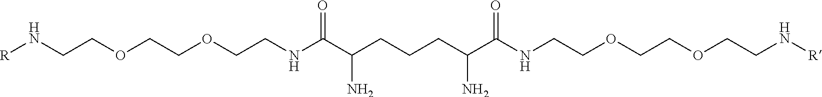 |
| 39 | 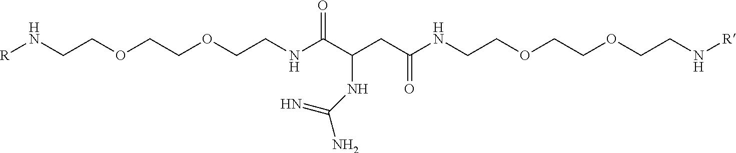 |
| 40 | 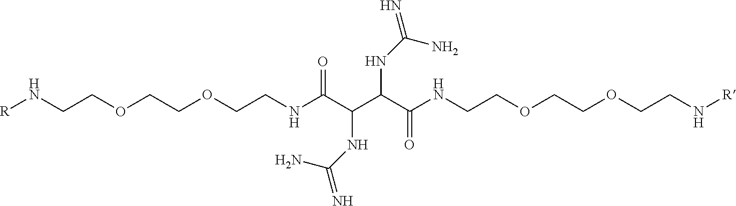 |
| 41 | 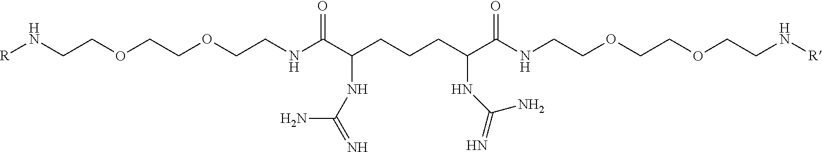 |
| 42 | 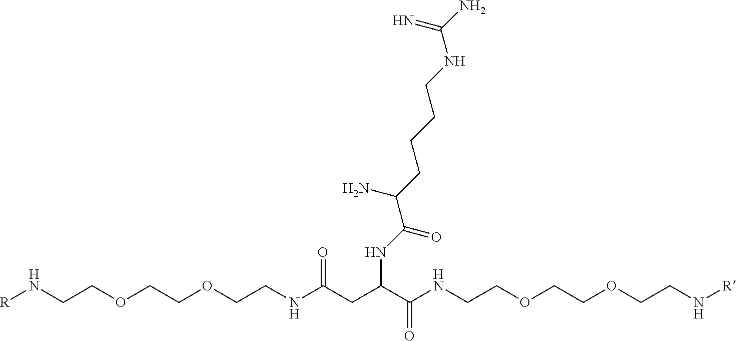 |
| 43 | 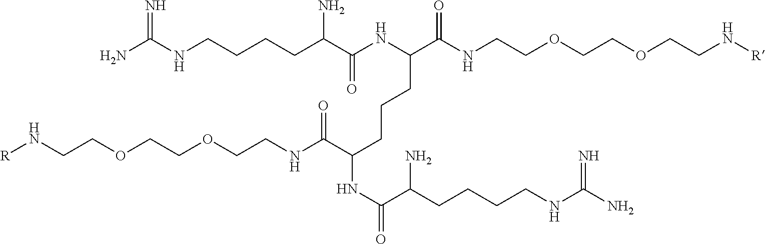 |

In another embodiment, provided herein are compounds of Formula (II), or pharmaceutically acceptable salts, solvates, polymorphs, prodrugs, metabolites, deuterides, N-oxides, stereoisomers, or isomers thereof:

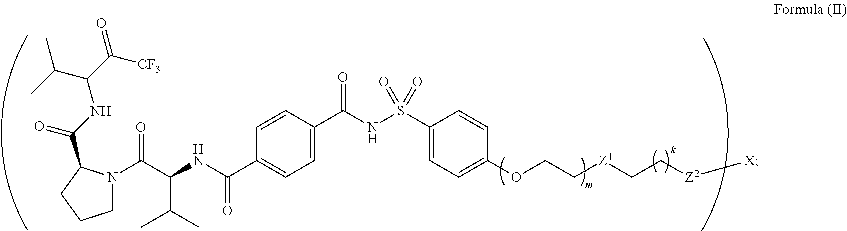

wherein:
X is alkylene, —C(O)—, —C(O)C(O)—,

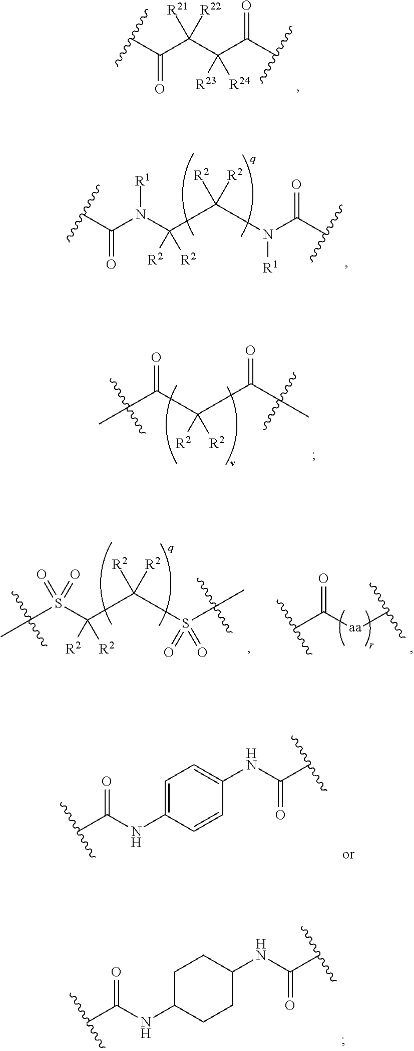

Z¹ is O or CR²R²;
Z² is O or NH;
aa is

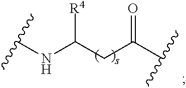

each R¹ is independently selected from H and alkyl;
each R² is independently selected from H, halo, —CN, —SR¹, alkyl, cycloalkyl, haloalkyl, —OR¹, and —R⁴;
each R⁴ is independently selected from alkyl, —CO₂R¹, -(alkylene)-(CO₂R¹), hydroxyalkyl, -(alkylene)(S(O)ₜ)(alkyl), -(alkylene)(NR⁵R⁵), and

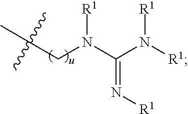

each R⁵ is independently selected from H, alkyl, haloalkyl, arylalkyl, heteroarylalkyl, aryl, and heteroaryl;
R²¹, R²², R²³, and R²⁴ are independently selected from H, halo, —CN, —SR¹, alkyl, cycloalkyl, haloalkyl, —NR¹R¹, and —OR¹;
each k is independently selected from 1, 2, 3, 4, 5, 6, 7, 8, 9, and 10;
each m is independently selected from 0, 1, 2, 3, 4, 5, 6, 7, 8, 9, and 10;
q is 1, 2, 3, 4, 5, 6, 7, 8, 9, or 10;
r is 3, 4, 5, 6, or 7;
each s is independently selected from 0, 1, 2, 3, and 4;
each t is independently selected from 0, 1, and 2; and
each u is independently selected from 1, 2, 3, 4, 5, 6, 7, 8, 9, and 10; and
v is selected from 3, 4, 5, 6, 7, 8, 9, and 10.

In some embodiments is a compound of Formula (II) wherein X is

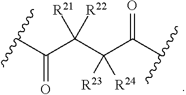

In some embodiments is a compound of Formula (II) wherein X is

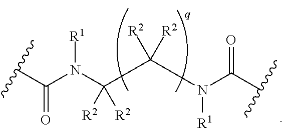

In some embodiments is a compound of Formula (II) wherein X is

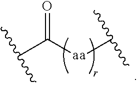

In some embodiments is a compound of Formula (II) wherein X is

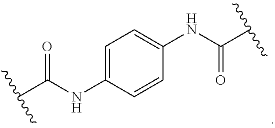 [SMILES: *C(=O)Nc1ccc(NC(*)=O)cc1]

In some embodiments is a compound of Formula (II) wherein X is

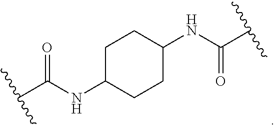 [SMILES: *C(=O)NC1CCC(NC(*)=O)CC1]

In some embodiments is a compound of Formula (II) wherein X is alkylene. In some embodiments is a compound of Formula (II) wherein X is —C(O)—. In some embodiments is a compound of Formula (II) wherein X is —C(O)C(O)—.

In some embodiments is a compound of Formula (II) wherein X is

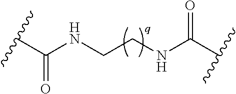

and q is 1. In some embodiments is a compound of Formula (II) wherein X is

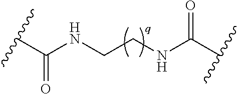

and q is 2. In some embodiments is a compound of Formula (II) wherein X is

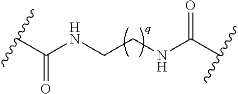

and q is 3. In some embodiments is a compound of Formula (II) wherein X is

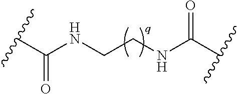

and q is 4. In some embodiments is a compound of Formula (II) wherein X is

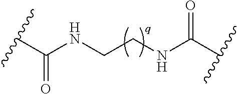

and q is 5. In some embodiments is a compound of Formula (II) wherein X is

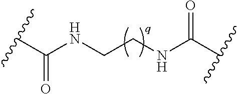

and q is 6. In some embodiments is a compound of Formula (II) wherein X is

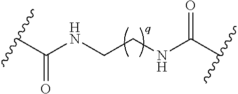

and q is 7.

In some embodiments is a compound of Formula (II) wherein Z¹ is O; and Z² is O. In some embodiments is a compound of Formula (II) wherein Z¹ is O; and Z² is NH. In some embodiments is a compound of Formula (II) wherein Z¹ is CH₂; and Z² is O. In some embodiments is a compound of Formula (II) wherein Z¹ is CH₂; and Z² is NH.

In some embodiments is a compound of Formula (II) wherein k is 1. In some embodiments is a compound of Formula (II) wherein k is 2. In some embodiments is a compound of Formula (II) wherein k is 3. In some embodiments is a compound of Formula (II) wherein k is 4. In some embodiments is a compound of Formula (II) wherein k is 5.

In some embodiments is a compound of Formula (II) wherein m is 0. In some embodiments is a compound of Formula (II) wherein m is 1. In some embodiments is a compound of Formula (II) wherein m is 2. In some embodiments is a compound of Formula (II) wherein m is 3. In some embodiments is a compound of Formula (II) wherein m is 4. In some embodiments is a compound of Formula (II) wherein m is 5. In some embodiments is a compound of Formula (II) wherein m is 6. In some embodiments is a compound of Formula (II) wherein m is 7.

In some embodiments is a compound of Formula (II) wherein X is

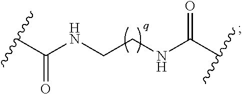

is 1, 2, 3, 4, 5, 6, 7, 8, 9, or 10; Z¹ is O; Z² is O; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (II) wherein X is

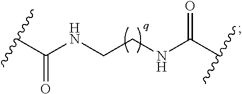

q is 1, 2, 3, 4, 5, or 6; Z¹ is O; Z² is O; each m is independently selected from 0, 1, 2, 3, 4, 5, and 6; and each k is independently selected from 1, 2, 3, 4, and 5. In some embodiments is a compound of Formula (II) wherein X is

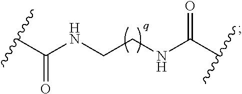

q is 3, 4, or 5; Z¹ is O; Z² is O; each m is independently selected from 1, 2, 3, 4, and 5; and each k is independently selected from 1, 2, and 3. In some embodiments is a compound of Formula (II) wherein X is

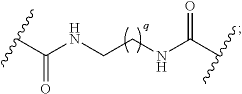

q is 3, 4, or 5; Z¹ is O; Z² is O; each m is independently selected from 1, 2, 3, 4, and 5; and each k is 1.

In some embodiments is a compound of Formula (II) wherein X is

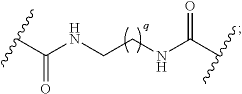

q is 1, 2, 3, 4, 5, 6, 7, 8, 9, or 10; Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (II) wherein X is

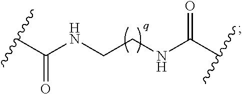

q is 1, 2, 3, 4, 5, or 6; Z¹ is O; Z² is NH; each m is independently selected from 0, 1, 2, 3, 4, 5, and 6; and each k is independently selected from 1, 2, 3, 4, and 5. In some embodiments is a compound of Formula (II) wherein X is

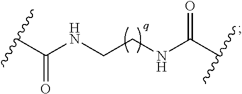

q is 3, 4, or 5; Z¹ is O; Z² is NH; each m is independently selected from 1, 2, 3, 4, and 5; and each k is independently selected from 1, 2, and 3. In some embodiments is a compound of Formula (II) wherein X is

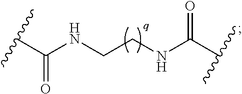

q is 3, 4, or 5; Z¹ is O; Z² is NH; each m is independently selected from 1, 2, 3, 4, and 5; and each k is 1.

In some embodiments is a compound of Formula (II) wherein X is

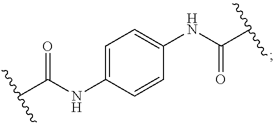 [SMILES: *C(=O)Nc1ccc(NC(*)=O)cc1]

Z¹ is O; Z² is O; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (II) wherein X is

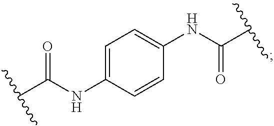 [SMILES: *C(=O)Nc1ccc(NC(*)=O)cc1]

Z¹ is O; Z² is O; each m is independently selected from 0, 1, 2, 3, 4, 5, and 6; and each k is independently selected from 1, 2, 3, 4, and 5. In some embodiments is a compound of Formula (II) wherein X is

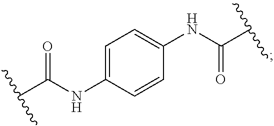 [SMILES: *C(=O)Nc1ccc(NC(*)=O)cc1]

Z¹ is O; Z² is O; each m is independently selected from 1, 2, 3, 4, and 5; and each k is independently selected from 1, 2, and 3. In some embodiments is a compound of Formula (II) wherein X is

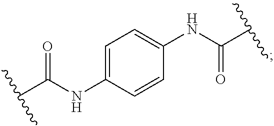 [SMILES: *C(=O)Nc1ccc(NC(*)=O)cc1]

Z¹ is O; Z² is O; each m is independently selected from 1, 2, 3, 4, and 5; and each k is 1.

In some embodiments is a compound of Formula (II) wherein X is

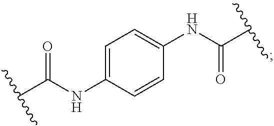 [SMILES: *C(=O)Nc1ccc(NC(*)=O)cc1]

Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (II) wherein X is

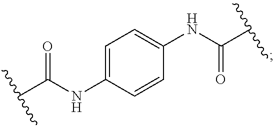 [SMILES: *C(=O)Nc1ccc(NC(*)=O)cc1]

Z¹ is O; Z² is NH; each m is independently selected from 0, 1, 2, 3, 4, 5, and 6; and each k is independently selected from 1, 2, 3, 4, and 5. In some embodiments is a compound of Formula (II) wherein X is

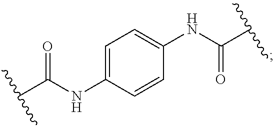 [SMILES: *C(=O)Nc1ccc(NC(*)=O)cc1]

Z¹ is O; Z² is NH; each m is independently selected from 1, 2, 3, 4, and 5; and each k is independently selected from 1, 2, and 3. In some embodiments is a compound of Formula (II) wherein X is

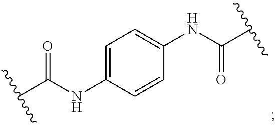 [SMILES: *C(=O)Nc1ccc(NC(*)=O)cc1]

Z¹ is O; Z² is NH; each m is independently selected from 1, 2, 3, 4, and 5; and each k is 1.

In some embodiments is a compound of Formula (II) wherein X is

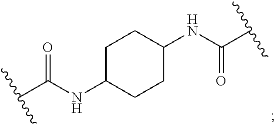 [SMILES: *C(=O)NC1CCC(NC(*)=O)CC1]

Z¹ is O; Z² is O; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (II) wherein X is

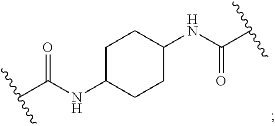 [SMILES: *C(=O)NC1CCC(NC(*)=O)CC1]

Z¹ is O; Z² is O; each m is independently selected from 0, 1, 2, 3, 4, 5, and 6; and each k is independently selected from 1, 2, 3, 4, and 5. In some embodiments is a compound of Formula (II) wherein X is

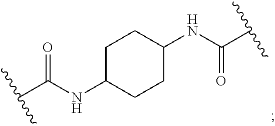 [SMILES: *C(=O)NC1CCC(NC(*)=O)CC1]

Z¹ is O; Z² is O; each m is independently selected from 1, 2, 3, 4, and 5; and each k is independently selected from 1, 2, and 3. In some embodiments is a compound of Formula (II) wherein X is

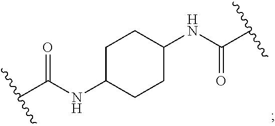 [SMILES: *C(=O)NC1CCC(NC(*)=O)CC1]

Z¹ is O; Z² is O; each m is independently selected from 1, 2, 3, 4, and 5; and each k is 1.

In some embodiments is a compound of Formula (II) wherein X is

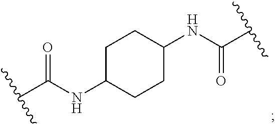 [SMILES: *C(=O)NC1CCC(NC(*)=O)CC1]

Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (II) wherein X is

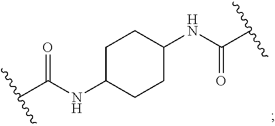 [SMILES: *C(=O)NC1CCC(NC(*)=O)CC1]

Z¹ is O; Z² is NH; each m is independently selected from 0, 1, 2, 3, 4, 5, and 6; and each k is independently selected from 1, 2, 3, 4, and 5. In some embodiments is a compound of Formula (II) wherein X is

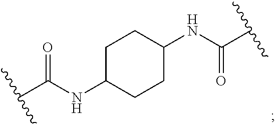 [SMILES: *C(=O)NC1CCC(NC(*)=O)CC1]

Z¹ is O; Z² is NH; each m is independently selected from 1, 2, 3, 4, and 5; and each k is independently selected from 1, 2, and 3. In some embodiments is a compound of Formula (II) wherein X is

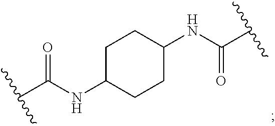 [SMILES: *C(=O)NC1CCC(NC(*)=O)CC1]

Z¹ is O; Z² is NH; each m is independently selected from 1, 2, 3, 4, and 5; and each k is 1.

In some embodiments is a compound of Formula (II) wherein X is alkylene; Z¹ is O; Z² is O; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (II) wherein X is alkylene; Z¹ is O; Z² is O; each m is independently selected from 0, 1, 2, 3, 4, 5, and 6; and each k is independently selected from 1, 2, 3, 4, and 5. In some embodiments is a compound of Formula (II) wherein X is alkylene; Z¹ is O; Z² is O; each m is independently selected from 1, 2, 3, 4, and 5; and each k is independently selected from 1, 2, and 3. In some embodiments is a compound of Formula (II) wherein X is alkylene; Z¹ is O; Z² is O; each m is independently selected from 1, 2, 3, 4, and 5; and each k is 1.

In some embodiments is a compound of Formula (II) wherein X is alkylene; Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (II) wherein X is alkylene; Z¹ is O; Z² is NH; each m is independently selected from 0, 1, 2, 3, 4, 5, and 6; and each k is independently selected from 1, 2, 3, 4, and 5. In some embodiments is a compound of Formula (II) wherein X is alkylene; Z¹ is O; Z² is NH; each m is independently selected from 1, 2, 3, 4, and 5; and each k is independently selected from 1, 2, and 3. In some embodiments is a compound of Formula (II) wherein X is alkylene; Z¹ is O; Z² is NH; each m is independently selected from 1, 2, 3, 4, and 5; and each k is 1.

In some embodiments is a compound of Formula (II) wherein X is —C(O)—; Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (II) wherein X is —C(O)—; Z¹ is O; Z² is NH; each m is independently selected from 0, 1, 2, 3, 4, 5, and 6; and each k is independently selected from 1, 2, 3, 4, and 5. In some embodiments is a compound of Formula (II) wherein X is —C(O)—; Z¹ is O; Z² is NH; each m is independently selected from 1, 2, 3, 4, and 5; and each k is independently selected from 1, 2, and 3. In some embodiments is a compound of Formula (II) wherein X is —C(O)—; Z¹ is O; Z² is NH; each m is independently selected from 1, 2, 3, 4, and 5; and each k is 1.

In some embodiments is a compound of Formula (II) wherein X is —C(O)C(O)—; Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (II) wherein X is —C(O)C(O)—; Z¹ is O; Z² is NH; each m is independently selected from 0, 1, 2, 3, 4, 5, and 6; and each k is independently selected from 1, 2, 3, 4, and 5. In some embodiments is a compound of Formula (II) wherein X is —C(O)C(O)—; Z¹ is O; Z² is NH; each m is independently selected from 1, 2, 3, 4, and 5; and each k is independently selected from 1, 2, and 3. In some embodiments is a compound of Formula (II) wherein X is —C(O)C(O)—; Z¹ is O; Z² is NH; each m is independently selected from 1, 2, 3, 4, and 5; and each k is 1.

In another embodiment, provided herein are compounds of Formula (III), or pharmaceutically acceptable salts, solvates, polymorphs, prodrugs, metabolites, deuterides, N-oxides, stereoisomers, or isomers thereof:

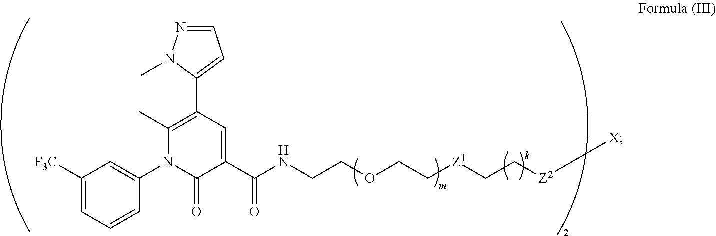

wherein:
X is alkylene, —C(O)—, —C(O)C(O)—,

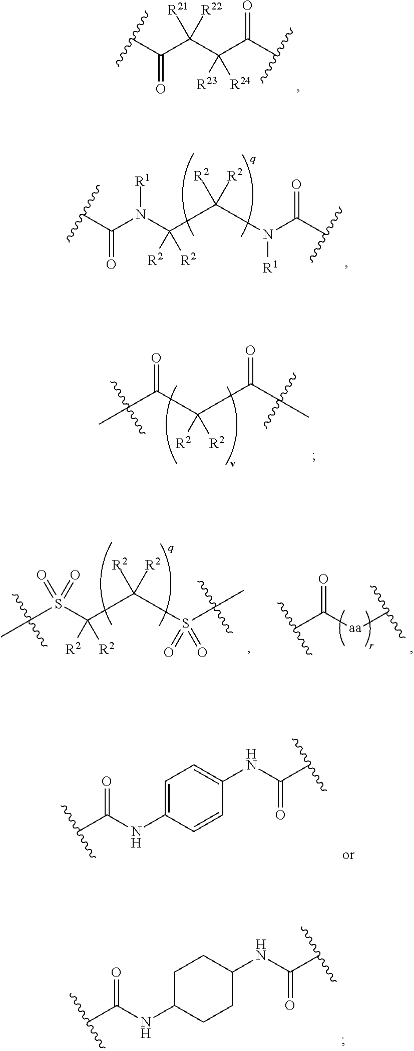

Z¹ is O or CR²R²;
Z² is O or NH;
aa is

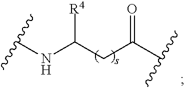

each R¹ is independently selected from H and alkyl;
each R² is independently selected from H, halo, —CN, —SR¹, alkyl, cycloalkyl, haloalkyl, —OR¹, and —R⁴;
each R⁴ is independently selected from alkyl, —CO₂R¹, -(alkylene)-(CO₂R¹), hydroxyalkyl, -(alkylene)(S(O)ₜ)(alkyl), -(alkylene)(NR⁵R⁵), and

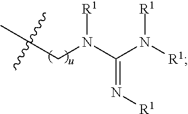

each R⁵ is independently selected from H, alkyl, haloalkyl, arylalkyl, heteroarylalkyl, aryl, and heteroaryl;
R²¹, R²², R²³, and R²⁴ are independently selected from H, halo, —CN, —SR¹, alkyl, cycloalkyl, haloalkyl, —NR¹R¹, and —OR¹;
each k is independently selected from 1, 2, 3, 4, 5, 6, 7, 8, 9, and 10;
each m is independently selected from 0, 1, 2, 3, 4, 5, 6, 7, 8, 9, and 10;
q is 1, 2, 3, 4, 5, 6, 7, 8, 9, or 10;
r is 3, 4, 5, 6, or 7;
each s is independently selected from 0, 1, 2, 3, and 4;
each t is independently selected from 0, 1, and 2;
each u is independently selected from 1, 2, 3, 4, 5, 6, 7, 8, 9, and 10; and
v is selected from 3, 4, 5, 6, 7, 8, 9, and 10.

In some embodiments is a compound of Formula (III) wherein X is

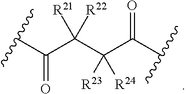

In some embodiments is a compound of Formula (III) wherein X is

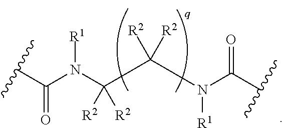

In some embodiments is a compound of Formula (III) wherein X is

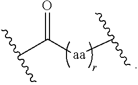

In some embodiments is a compound of Formula (III) wherein X is

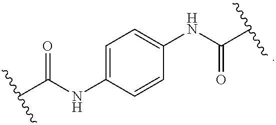 [SMILES: *C(=O)Nc1ccc(NC(*)=O)cc1]

In some embodiments is a compound of Formula (III) wherein X is

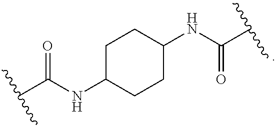 [SMILES: *C(=O)NC1CCC(NC(*)=O)CC1]

In some embodiments is a compound of Formula (III) wherein X is alkylene. In some embodiments is a compound of Formula (III) wherein X is —C(O)—. In some embodiments is a compound of Formula (III) wherein X is —C(O)C(O)—.

In some embodiments is a compound of Formula (III) wherein X is

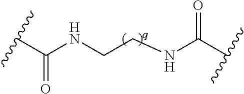

and q is 1. In some embodiments is a compound of Formula (III) wherein X is

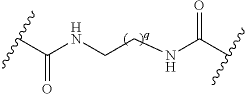

and q is 2. In some embodiments is a compound of Formula (III) wherein X is

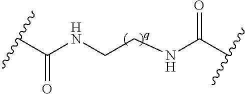

and q is 3. In some embodiments is a compound of Formula (III) wherein X is

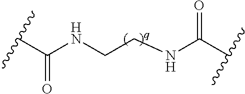

and q is 4. In some embodiments is a compound of Formula (III) wherein X is

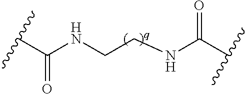

and q is 5. In some embodiments is a compound of Formula (III) wherein X is

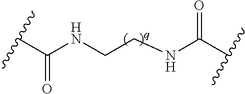

and q is 6. In some embodiments is a compound of Formula (III) wherein X is

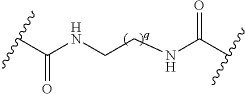

and q is 7.

In some embodiments is a compound of Formula (III) wherein Z¹ is O; and Z² is O. In some embodiments is a compound of Formula (III) wherein Z¹ is O; and Z² is NH. In some embodiments is a compound of Formula (III) wherein Z¹ is CH₂; and Z² is O. In some embodiments is a compound of Formula (III) wherein Z¹ is CH₂; and Z² is NH.

In some embodiments is a compound of Formula (III) wherein k is 1. In some embodiments is a compound of Formula (III) wherein k is 2. In some embodiments is a compound of Formula (III) wherein k is 3. In some embodiments is a compound of Formula (III) wherein k is 4. In some embodiments is a compound of Formula (III) wherein k is 5.

In some embodiments is a compound of Formula (III) wherein m is 0. In some embodiments is a compound of Formula (III) wherein m is 1. In some embodiments is a compound of Formula (III) wherein m is 2. In some embodiments is a compound of Formula (III) wherein m is 3. In some embodiments is a compound of Formula (III) wherein m is 4. In some embodiments is a compound of Formula (III) wherein m is 5. In some embodiments is a compound of Formula (III) wherein m is 6. In some embodiments is a compound of Formula (III) wherein m is 7.

In some embodiments is a compound of Formula (III) wherein X is

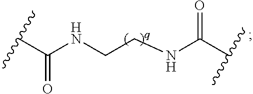

q is 1, 2, 3, 4, 5, 6, 7, 8, 9, or 10; Z¹ is O; Z² is O; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (III) wherein X is

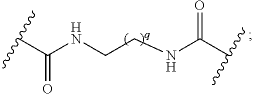

q is 1, 2, 3, 4, 5, or 6; Z¹ is O; Z² is O; each m is independently selected from 0, 1, 2, 3, 4, 5, and 6; and each k is independently selected from 1, 2, 3, 4, and 5. In some embodiments is a compound of Formula (III) wherein X is

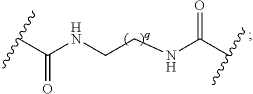

q is 3, 4, or 5; Z¹ is O; Z² is O; each m is independently selected from 1, 2, 3, 4, and 5; and each k is independently selected from 1, 2, and 3. In some embodiments is a compound of Formula (III) wherein X is

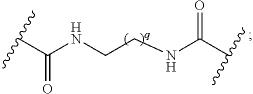

q is 3, 4, or 5; Z¹ is O; Z² is O; each m is independently selected from 1, 2, 3, 4, and 5; and each k is 1.

In some embodiments is a compound of Formula (III) wherein X is

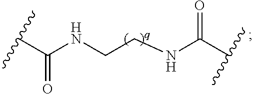

q is 1, 2, 3, 4, 5, 6, 7, 8, 9, or 10; Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (III) wherein X is

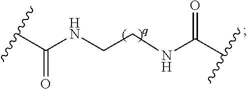

q is 1, 2, 3, 4, 5, or 6; Z¹ is O; Z² is NH; each m is independently selected from 0, 1, 2, 3, 4, 5, and 6; and each k is independently selected from 1, 2, 3, 4, and 5. In some embodiments is a compound of Formula (III) wherein X is

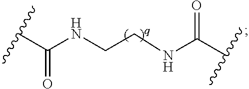

q is 3, 4, or 5; Z¹ is O; Z² is NH; each m is independently selected from 1, 2, 3, 4, and 5; and each k is independently selected from 1, 2, and 3. In some embodiments is a compound of Formula (III) wherein X is

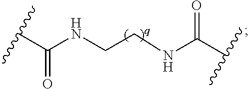

q is 3, 4, or 5; Z¹ is O; Z² is NH; each m is independently selected from 1, 2, 3, 4, and 5; and each k is 1.

In some embodiments is a compound of Formula (III) wherein X is

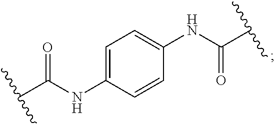 [SMILES: *C(=O)Nc1ccc(NC(*)=O)cc1]

Z¹ is O; Z² is O; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (III) wherein X is

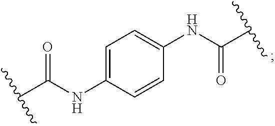 [SMILES: *C(=O)Nc1ccc(NC(*)=O)cc1]

Z¹ is O; Z² is O; each m is independently selected from 0, 1, 2, 3, 4, 5, and 6; and each k is independently selected from 1, 2, 3, 4, and 5. In some embodiments is a compound of Formula (III) wherein X is

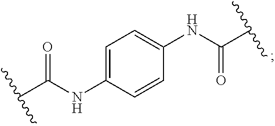 [SMILES: *C(=O)Nc1ccc(NC(*)=O)cc1]

Z¹ is O; Z² is O; each m is independently selected from 1, 2, 3, 4, and 5; and each k is independently selected from 1, 2, and 3. In some embodiments is a compound of Formula (III) wherein X is

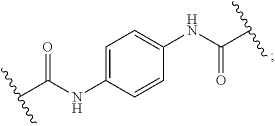 [SMILES: *C(=O)Nc1ccc(NC(*)=O)cc1]

Z¹ is O; Z² is O; each m is independently selected from 1, 2, 3, 4, and 5; and each k is 1.

In some embodiments is a compound of Formula (III) wherein X is

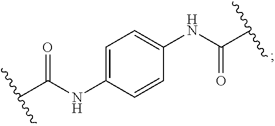 [SMILES: *C(=O)Nc1ccc(NC(*)=O)cc1]

Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (III) wherein X is

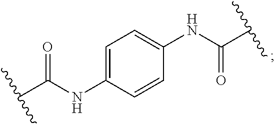 [SMILES: *C(=O)Nc1ccc(NC(*)=O)cc1]

Z¹ is O; Z² is NH; each m is independently selected from 0, 1, 2, 3, 4, 5, and 6; and each k is independently selected from 1, 2, 3, 4, and 5. In some embodiments is a compound of Formula (III) wherein X is

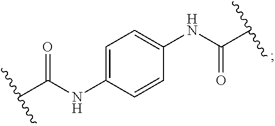 [SMILES: *C(=O)Nc1ccc(NC(*)=O)cc1]

Z¹ is O; Z² is NH; each m is independently selected from 1, 2, 3, 4, and 5; and each k is independently selected from 1, 2, and 3. In some embodiments is a compound of Formula (III) wherein X is

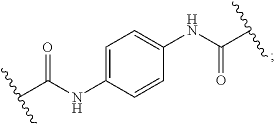 [SMILES: *C(=O)Nc1ccc(NC(*)=O)cc1]

Z¹ is O; Z² is NH; each m is independently selected from 1, 2, 3, 4, and 5; and each k is 1.

In some embodiments is a compound of Formula (III) wherein X is

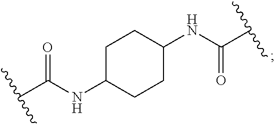 [SMILES: *C(=O)NC1CCC(NC(*)=O)CC1]

Z¹ is O; Z² is O; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (III) wherein X is

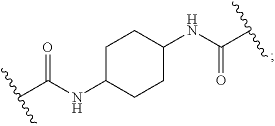 [SMILES: *C(=O)NC1CCC(NC(*)=O)CC1]

Z¹ is O; Z² is O; each m is independently selected from 0, 1, 2, 3, 4, 5, and 6; and each k is independently selected from 1, 2, 3, 4, and 5. In some embodiments is a compound of Formula (III) wherein X is

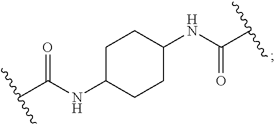 [SMILES: *C(=O)NC1CCC(NC(*)=O)CC1]

Z¹ is O; Z² is O; each m is independently selected from 1, 2, 3, 4, and 5; and each k is independently selected from 1, 2, and 3. In some embodiments is a compound of Formula (III) wherein X is

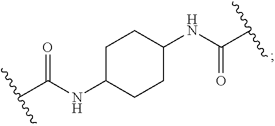 [SMILES: *C(=O)NC1CCC(NC(*)=O)CC1]

Z¹ is O; Z² is O; each m is independently selected from 1, 2, 3, 4, and 5; and each k is 1.

In some embodiments is a compound of Formula (III) wherein X is

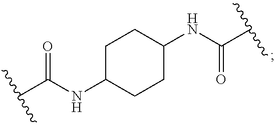 [SMILES: *C(=O)NC1CCC(NC(*)=O)CC1]

Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (III) wherein X is

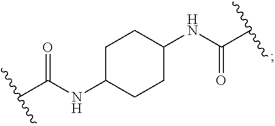 [SMILES: *C(=O)NC1CCC(NC(*)=O)CC1]

Z¹ is O; Z² is NH; each m is independently selected from 0, 1, 2, 3, 4, 5, and 6; and each k is independently selected from 1, 2, 3, 4, and 5. In some embodiments is a compound of Formula (III) wherein X is

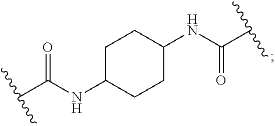 [SMILES: *C(=O)NC1CCC(NC(*)=O)CC1]

Z¹ is O; Z² is NH; each m is independently selected from 1, 2, 3, 4, and 5; and each k is independently selected from 1, 2, and 3. In some embodiments is a compound of Formula (III) wherein X is

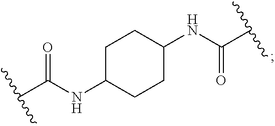 [SMILES: *C(=O)NC1CCC(NC(*)=O)CC1]

Z¹ is O; Z² is NH; each m is independently selected from 1, 2, 3, 4, and 5; and each k is 1.

In some embodiments is a compound of Formula (III) wherein X is alkylene; Z¹ is O; Z² is O; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (III) wherein X is alkylene; Z¹ is O; Z² is O; each m is independently selected from 0, 1, 2, 3, 4, 5, and 6; and each k is independently selected from 1, 2, 3, 4, and 5. In some embodiments is a compound of Formula (III) wherein X is alkylene; Z¹ is O; Z² is O; each m is independently selected from 1, 2, 3, 4, and 5; and each k is independently selected from 1, 2, and 3. In some embodiments is a compound of Formula (III) wherein X is alkylene; Z¹ is O; Z² is O; each m is independently selected from 1, 2, 3, 4, and 5; and each k is 1.

In some embodiments is a compound of Formula (III) wherein X is alkylene; Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (III) wherein X is alkylene; Z¹ is O; Z² is NH; each m is independently selected from 0, 1, 2, 3, 4, 5, and 6; and each k is independently selected from 1, 2, 3, 4, and 5. In some embodiments is a compound of Formula (III) wherein X is alkylene; Z¹ is O; Z² is NH; each m is independently selected from 1, 2, 3, 4, and 5; and each k is independently selected from 1, 2, and 3. In some embodiments is a compound of Formula (III) wherein X is alkylene; Z¹ is O; Z² is NH; each m is independently selected from 1, 2, 3, 4, and 5; and each k is 1.

In some embodiments is a compound of Formula (III) wherein X is —C(O)—; Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (III) wherein X is —C(O)—; Z¹ is O; Z² is NH; each m is independently selected from 0, 1, 2, 3, 4, 5, and 6; and each k is independently selected from 1, 2, 3, 4, and 5. In some embodiments is a compound of Formula (III) wherein X is —C(O)—; Z¹ is O; Z² is NH; each m is independently selected from 1, 2, 3, 4, and 5; and each k is independently selected from 1, 2, and 3. In some embodiments is a compound of Formula (III) wherein X is —C(O)—; Z¹ is O; Z² is NH; each m is independently selected from 1, 2, 3, 4, and 5; and each k is 1.

In some embodiments is a compound of Formula (III) wherein X is —C(O)C(O)—; Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (III) wherein X is —C(O)C(O)—; Z¹ is O; Z² is NH; each m is independently selected from 0, 1, 2, 3, 4, 5, and 6; and each k is independently selected from 1, 2, 3, 4, and 5. In some embodiments is a compound of Formula (III) wherein X is —C(O)C(O)—; Z¹ is O; Z² is NH; each m is independently selected from 1, 2, 3, 4, and 5; and each k is independently selected from 1, 2, and 3. In some embodiments is a compound of Formula (III) wherein X is —C(O)C(O)—; Z¹ is O; Z² is NH; each m is independently selected from 1, 2, 3, 4, and 5; and each k is 1.

In another embodiment, provided herein are compounds of Formula (IV), or pharmaceutically acceptable salts, solvates, polymorphs, prodrugs, metabolites, deuterides, N-oxides, stereoisomers, or isomers thereof:

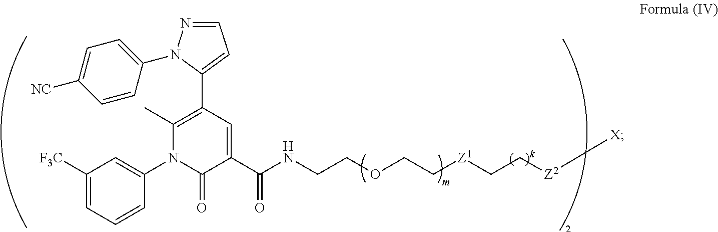

wherein:
X is alkylene, —C(O)—, —C(O)C(O)—,

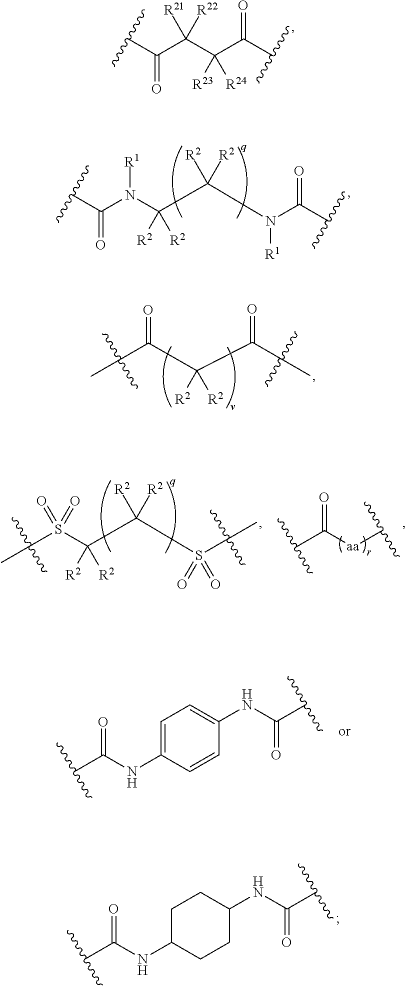

Z¹ is O or CR²R²;
Z² is O or NH;
aa is

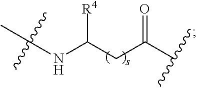

each R¹ is independently selected from H and alkyl;
each R² is independently selected from H, halo, —CN, —SR¹, alkyl, cycloalkyl, haloalkyl, —OR¹, and —R⁴;
each R⁴ is independently selected from alkyl, —CO₂R¹, -(alkylene)-(CO₂R¹), hydroxyalkyl, -(alkylene)(S(O)ₜ)(alkyl), -(alkylene)(NR⁵R⁵), and

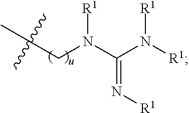

each R⁵ is independently selected from H, alkyl, haloalkyl, arylalkyl, heteroarylalkyl, aryl, and heteroaryl;
R²¹, R²², R²³, and R²⁴ are independently selected from H, halo, —CN, —SR¹, alkyl, cycloalkyl, haloalkyl, —NR¹R¹, and —OR¹;
each k is independently selected from 1, 2, 3, 4, 5, 6, 7, 8, 9, and 10;
each m is independently selected from 0, 1, 2, 3, 4, 5, 6, 7, 8, 9, and 10;
q is 1, 2, 3, 4, 5, 6, 7, 8, 9, or 10;
r is 3, 4, 5, 6, or 7;
each s is independently selected from 0, 1, 2, 3, and 4;
each t is independently selected from 0, 1, and 2;
each u is independently selected from 1, 2, 3, 4, 5, 6, 7, 8, 9, and 10; and
v is selected from 3, 4, 5, 6, 7, 8, 9, and 10.

In some embodiments is a compound of Formula (IV) wherein X is

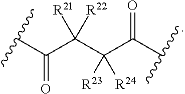

In some embodiments is a compound of Formula (IV) wherein X is

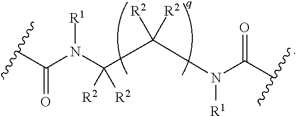

In some embodiments is a compound of Formula (IV) wherein X is

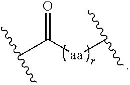

In some embodiments is a compound of Formula (IV) wherein X is

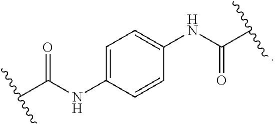 [SMILES: *C(=O)Nc1ccc(NC(*)=O)cc1]

In some embodiments is a compound of Formula (IV) wherein X is

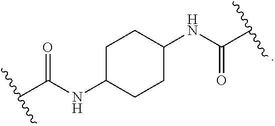 [SMILES: *C(=O)NC1CCC(NC(*)=O)CC1]

In some embodiments is a compound of Formula (IV) wherein X is alkylene. In some embodiments is a compound of Formula (IV) wherein X is —C(O)—. In some embodiments is a compound of Formula (IV) wherein X is —C(O)C(O)—.

In some embodiments is a compound of Formula (IV) wherein X is

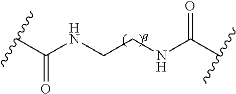

and q is 1. In some embodiments is a compound of Formula (IV) wherein X is

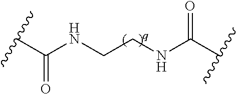

and q is 2. In some embodiments is a compound of Formula (IV) wherein X is

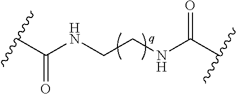

and q is 3. In some embodiments is a compound of Formula (IV) wherein X is

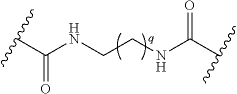

and q is 4. In some embodiments is a compound of Formula (IV) wherein X is

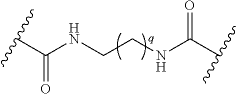

and q is 5. In some embodiments is a compound of Formula (IV) wherein X is

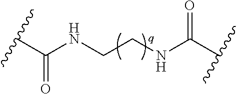

and q is 6. In some embodiments is a compound of Formula (IV) wherein X is

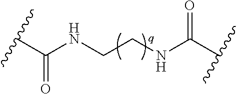

and q is 7.

In some embodiments is a compound of Formula (IV) wherein Z¹ is O; and Z² is O. In some embodiments is a compound of Formula (IV) wherein Z¹ is O; and Z² is NH. In some embodiments is a compound of Formula (IV) wherein Z¹ is CH₂; and Z² is O. In some embodiments is a compound of Formula (IV) wherein Z¹ is CH₂; and Z² is NH.

In some embodiments is a compound of Formula (IV) wherein k is 1. In some embodiments is a compound of Formula (IV) wherein k is 2. In some embodiments is a compound of Formula (IV) wherein k is 3. In some embodiments is a compound of Formula (IV) wherein k is 4. In some embodiments is a compound of Formula (IV) wherein k is 5.

In some embodiments is a compound of Formula (IV) wherein m is 0. In some embodiments is a compound of Formula (IV) wherein m is 1. In some embodiments is a compound of Formula (IV) wherein m is 2. In some embodiments is a compound of Formula (IV) wherein m is 3. In some embodiments is a compound of Formula (IV) wherein m is 4. In some embodiments is a compound of Formula (IV) wherein m is 5. In some embodiments is a compound of Formula (IV) wherein m is 6. In some embodiments is a compound of Formula (IV) wherein m is 7.

In some embodiments is a compound of Formula (IV) wherein X is

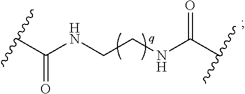

q is 1, 2, 3, 4, 5, 6, 7, 8, 9, or 10; Z¹ is O; Z² is O; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (IV) wherein X is

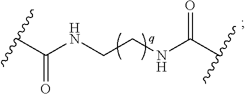

q is 1, 2, 3, 4, 5, or 6; Z¹ is O; Z² is O; each m is independently selected from 0, 1, 2, 3, 4, 5, and 6; and each k is independently selected from 1, 2, 3, 4, and 5. In some embodiments is a compound of Formula (IV) wherein X is

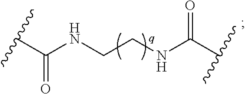

q is 3, 4, or 5; Z¹ is O; Z² is O; each m is independently selected from 1, 2, 3, 4, and 5; and each k is independently selected from 1, 2, and 3. In some embodiments is a compound of Formula (IV) wherein X is

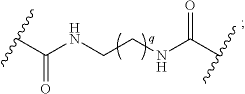

q is 3, 4, or 5; Z¹ is O; Z² is O; each m is independently selected from 1, 2, 3, 4, and 5; and each k is 1.

In some embodiments is a compound of Formula (IV) wherein X is

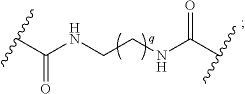

q is 1, 2, 3, 4, 5, 6, 7, 8, 9, or 10; Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (IV) wherein X is

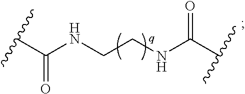

q is 1, 2, 3, 4, 5, or 6; Z¹ is O; Z² is NH; each m is independently selected from 0, 1, 2, 3, 4, 5, and 6; and each k is independently selected from 1, 2, 3, 4, and 5. In some embodiments is a compound of Formula (IV) wherein X is

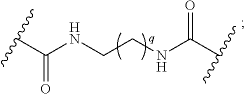

q is 3, 4, or 5; Z¹ is O; Z² is NH; each m is independently selected from 1, 2, 3, 4, and 5; and each k is independently selected from 1, 2, and 3. In some embodiments is a compound of Formula (IV) wherein X is

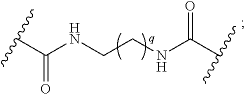

q is 3, 4, or 5; Z¹ is O; Z² is NH; each m is independently selected from 1, 2, 3, 4, and 5; and each k is 1.

In some embodiments is a compound of Formula (IV) wherein X is

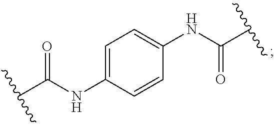 [SMILES: *C(=O)Nc1ccc(NC(*)=O)cc1]

Z¹ is O; Z² is O; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (IV) wherein X is

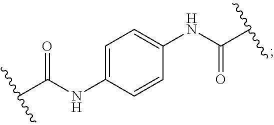 [SMILES: *C(=O)Nc1ccc(NC(*)=O)cc1]

Z¹ is O; Z² is O; each m is independently selected from 0, 1, 2, 3, 4, 5, and 6; and each k is independently selected from 1, 2, 3, 4, and 5. In some embodiments is a compound of Formula (IV) wherein X is

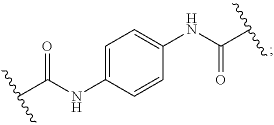 [SMILES: *C(=O)Nc1ccc(NC(*)=O)cc1]

Z¹ is O; Z² is O; each m is independently selected from 1, 2, 3, 4, and 5; and each k is independently selected from 1, 2, and 3. In some embodiments is a compound of Formula (IV) wherein X is

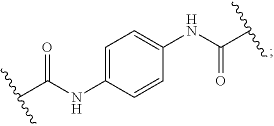 [SMILES: *C(=O)Nc1ccc(NC(*)=O)cc1]

Z¹ is O; Z² is O; each m is independently selected from 1, 2, 3, 4, and 5; and each k is 1.

In some embodiments is a compound of Formula (IV) wherein X is

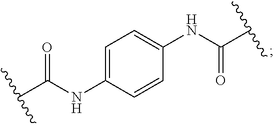 [SMILES: *C(=O)Nc1ccc(NC(*)=O)cc1]

Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (IV) wherein X is

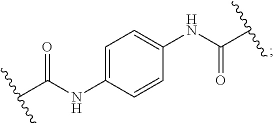 [SMILES: *C(=O)Nc1ccc(NC(*)=O)cc1]

Z¹ is O; Z² is NH; each m is independently selected from 0, 1, 2, 3, 4, 5, and 6; and each k is independently selected from 1, 2, 3, 4, and 5. In some embodiments is a compound of Formula (IV) wherein X is

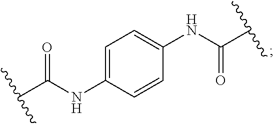 [SMILES: *C(=O)Nc1ccc(NC(*)=O)cc1]

Z¹ is O; Z² is NH; each m is independently selected from 1, 2, 3, 4, and 5; and each k is independently selected from 1, 2, and 3. In some embodiments is a compound of Formula (IV) wherein X is

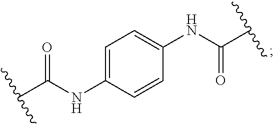 [SMILES: *C(=O)Nc1ccc(NC(*)=O)cc1]

Z¹ is O; Z² is NH; each m is independently selected from 1, 2, 3, 4, and 5; and each k is 1.

In some embodiments is a compound of Formula (IV) wherein X is

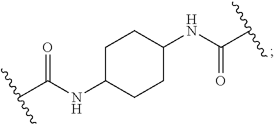 [SMILES: *C(=O)NC1CCC(NC(*)=O)CC1]

Z¹ is O; Z² is O; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (IV) wherein X is

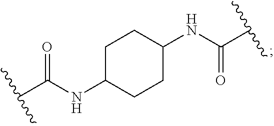 [SMILES: *C(=O)NC1CCC(NC(*)=O)CC1]

Z¹ is O; Z² is O; each m is independently selected from 0, 1, 2, 3, 4, 5, and 6; and each k is independently selected from 1, 2, 3, 4, and 5. In some embodiments is a compound of Formula (IV) wherein X is

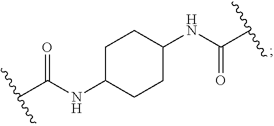 [SMILES: *C(=O)NC1CCC(NC(*)=O)CC1]

Z¹ is O; Z² is O; each m is independently selected from 1, 2, 3, 4, and 5; and each k is independently selected from 1, 2, and 3. In some embodiments is a compound of Formula (IV) wherein X is

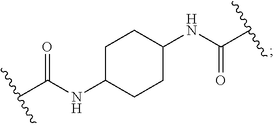 [SMILES: *C(=O)NC1CCC(NC(*)=O)CC1]

Z¹ is O; Z² is O; each m is independently selected from 1, 2, 3, 4, and 5; and each k is 1.

In some embodiments is a compound of Formula (IV) wherein X is

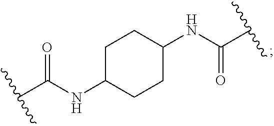 [SMILES: *C(=O)NC1CCC(NC(*)=O)CC1]

Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (IV) wherein X is

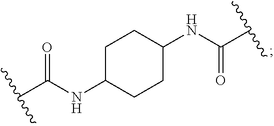 [SMILES: *C(=O)NC1CCC(NC(*)=O)CC1]

Z¹ is O; Z² is NH; each m is independently selected from 0, 1, 2, 3, 4, 5, and 6; and each k is independently selected from 1, 2, 3, 4, and 5. In some embodiments is a compound of Formula (IV) wherein X is

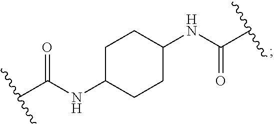 [SMILES: *C(=O)NC1CCC(NC(*)=O)CC1]

Z¹ is O; Z² is NH; each m is independently selected from 1, 2, 3, 4, and 5; and each k is independently selected from 1, 2, and 3. In some embodiments is a compound of Formula (IV) wherein X is

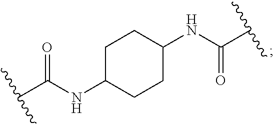 [SMILES: *C(=O)NC1CCC(NC(*)=O)CC1]

Z¹ is O; Z² is NH; each m is independently selected from 1, 2, 3, 4, and 5; and each k is 1.

In some embodiments is a compound of Formula (IV) wherein X is alkylene; Z¹ is O; Z² is O; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (IV) wherein X is alkylene; Z¹ is O; Z² is O; each m is independently selected from 0, 1, 2, 3, 4, 5, and 6; and each k is independently selected from 1, 2, 3, 4, and 5. In some embodiments is a compound of Formula (IV) wherein X is alkylene; Z¹ is O; Z² is O; each m is independently selected from 1, 2, 3, 4, and 5; and each k is independently selected from 1, 2, and 3. In some embodiments is a compound of Formula (IV) wherein X is alkylene; Z¹ is O; Z² is O; each m is independently selected from 1, 2, 3, 4, and 5; and each k is 1.

In some embodiments is a compound of Formula (IV) wherein X is alkylene; Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (IV) wherein X is alkylene; Z¹ is O; Z² is NH; each m is independently selected from 0, 1, 2, 3, 4, 5, and 6; and each k is independently selected from 1, 2, 3, 4, and 5. In some embodiments is a compound of Formula (IV) wherein X is alkylene; Z¹ is O; Z² is NH; each m is independently selected from 1, 2, 3, 4, and 5; and each k is independently selected from 1, 2, and 3. In some embodiments is a compound of Formula (IV) wherein X is alkylene; Z¹ is O; Z² is NH; each m is independently selected from 1, 2, 3, 4, and 5; and each k is 1.

In some embodiments is a compound of Formula (IV) wherein X is —C(O)—; Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (IV) wherein X is —C(O)—; Z¹ is O; Z² is NH; each m is independently selected from 0, 1, 2, 3, 4, 5, and 6; and each k is independently selected from 1, 2, 3, 4, and 5. In some embodiments is a compound of Formula (IV) wherein X is —C(O)—; Z¹ is O; Z² is NH; each m is independently selected from 1, 2, 3, 4, and 5; and each k is independently selected from 1, 2, and 3. In some embodiments is a compound of Formula (IV) wherein X is —C(O)—; Z¹ is O; Z² is NH; each m is independently selected from 1, 2, 3, 4, and 5; and each k is 1.

In some embodiments is a compound of Formula (IV) wherein X is —C(O)C(O)—; Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (IV) wherein X is —C(O)C(O)—; Z¹ is O; Z² is NH; each m is independently selected from 0, 1, 2, 3, 4, 5, and 6; and each k is independently selected from 1, 2, 3, 4, and 5. In some embodiments is a compound of Formula (IV) wherein X is —C(O)C(O)—; Z¹ is O; Z² is NH; each m is independently selected from 1, 2, 3, 4, and 5; and each k is independently selected from 1, 2, and 3. In some embodiments is a compound of Formula (IV) wherein X is —C(O)C(O)—; Z¹ is O; Z² is NH; each m is independently selected from 1, 2, 3, 4, and 5; and each k is 1.

In another embodiment, provided herein are compounds of Formula (V), or pharmaceutically acceptable salts, solvates, polymorphs, prodrugs, metabolites, deuterides, N-oxides, stereoisomers, or isomers thereof:

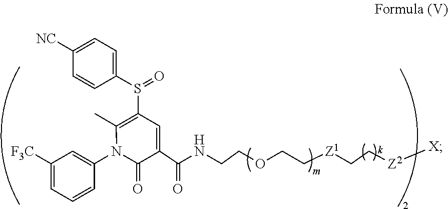

wherein:
X is alkylene, —C(O)—, —C(O)C(O)—,

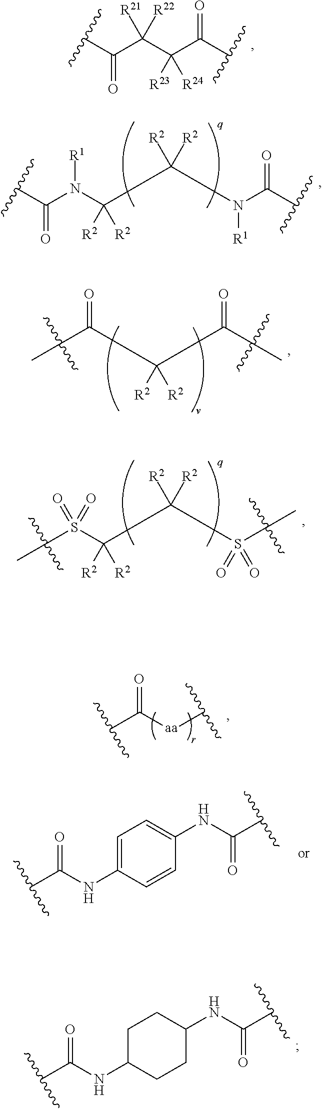

Z¹ is O or CR²R²;
Z² is O or NH;
aa is

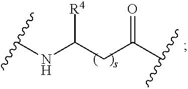

each R¹ is independently selected from H and alkyl;
each R² is independently selected from H, halo, —CN, —SR¹, alkyl, cycloalkyl, haloalkyl, —OR¹, and —R⁴;
each R⁴ is independently selected from alkyl, —CO₂R¹, -(alkylene)-(CO₂R¹), hydroxyalkyl, -(alkylene)(S(O)ₜ)(alkyl), -(alkylene)(NR⁵R⁵), and

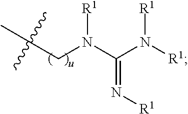

each R⁵ is independently selected from H, alkyl, haloalkyl, arylalkyl, heteroarylalkyl, aryl, and heteroaryl;
R²¹, R²², R²³, and R²⁴ are independently selected from H, halo, —CN, —SR¹, alkyl, cycloalkyl, haloalkyl, —NR¹R¹, and —OR¹;
each k is independently selected from 1, 2, 3, 4, 5, 6, 7, 8, 9, and 10;
each m is independently selected from 0, 1, 2, 3, 4, 5, 6, 7, 8, 9, and 10;
q is 1, 2, 3, 4, 5, 6, 7, 8, 9, or 10;
r is 3, 4, 5, 6, or 7;
each s is independently selected from 0, 1, 2, 3, and 4;
each t is independently selected from 0, 1, and 2;
each u is independently selected from 1, 2, 3, 4, 5, 6, 7, 8, 9, and 10; and
v is selected from 3, 4, 5, 6, 7, 8, 9, and 10.

In some embodiments is a compound of Formula (V) wherein X is

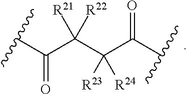

In some embodiments is a compound of Formula (V) wherein X is

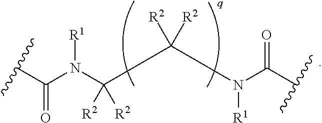

In some embodiments is a compound of Formula (V) wherein X is

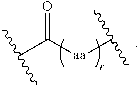

In some embodiments is a compound of Formula (V) wherein X is

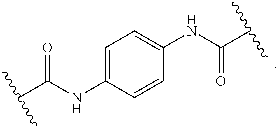 [SMILES: *C(=O)Nc1ccc(NC(*)=O)cc1]

In some embodiments is a compound of Formula (V) wherein X is

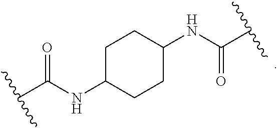 [SMILES: *C(=O)NC1CCC(NC(*)=O)CC1]

In some embodiments is a compound of Formula (V) wherein X is alkylene. In some embodiments is a compound of Formula (V) wherein X is —C(O)—. In some embodiments is a compound of Formula (V) wherein X is —C(O)C(O)—.

In some embodiments is a compound of Formula (V) wherein X is

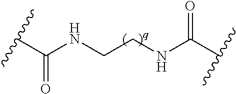

and q is 1. In some embodiments is a compound of Formula (V) wherein X is

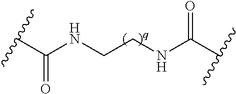

and q is 2. In some embodiments is a compound of Formula (V) wherein X is

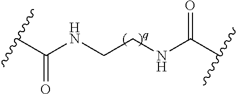

and q is 3. In some embodiments is a compound of Formula (V) wherein X is

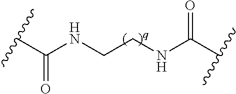

and q is 4. In some embodiments is a compound of Formula (V) wherein X is

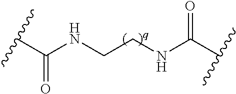

and q is 5. In some embodiments is a compound of Formula (V) wherein X is

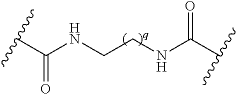

and q is 6. In some embodiments is a compound of Formula (V) wherein X is

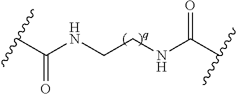

and q is 7.

In some embodiments is a compound of Formula (V) wherein Z¹ is O; and Z² is O. In some embodiments is a compound of Formula (V) wherein Z¹ is O; and Z² is NH. In some embodiments is a compound of Formula (V) wherein Z¹ is CH₂; and Z² is O. In some embodiments is a compound of Formula (V) wherein Z¹ is CH₂; and Z² is NH.

In some embodiments is a compound of Formula (V) wherein k is 1. In some embodiments is a compound of Formula (V) wherein k is 2. In some embodiments is a compound of Formula (V) wherein k is 3. In some embodiments is a compound of Formula (V) wherein k is 4. In some embodiments is a compound of Formula (V) wherein k is 5.

In some embodiments is a compound of Formula (V) wherein m is 0. In some embodiments is a compound of Formula (V) wherein m is 1. In some embodiments is a compound of Formula (V) wherein m is 2. In some embodiments is a compound of Formula (V) wherein m is 3. In some embodiments is a compound of Formula (V) wherein m is 4. In some embodiments is a compound of Formula (V) wherein m is 5. In some embodiments is a compound of Formula (V) wherein m is 6. In some embodiments is a compound of Formula (V) wherein m is 7.

In some embodiments is a compound of Formula (V) wherein X is

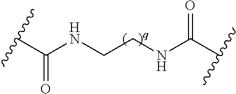

q is 1, 2, 3, 4, 5, 6, 7, 8, 9, or 10; Z¹ is O; Z² is O; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (V) wherein X is

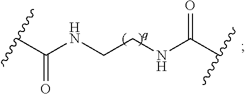

q is 1, 2, 3, 4, 5, or 6; Z¹ is O; Z² is O; each m is independently selected from 0, 1, 2, 3, 4, 5, and 6; and each k is independently selected from 1, 2, 3, 4, and 5. In some embodiments is a compound of Formula (V) wherein X is

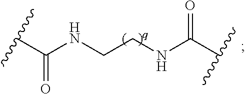

q is 3, 4, or 5; Z¹ is O; Z² is O; each m is independently selected from 1, 2, 3, 4, and 5; and each k is independently selected from 1, 2, and 3. In some embodiments is a compound of Formula (V) wherein X is

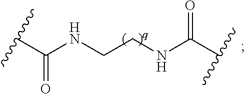

q is 3, 4, or 5; Z¹ is O; Z² is O; each m is independently selected from 1, 2, 3, 4, and 5; and each k is 1.

In some embodiments is a compound of Formula (V) wherein X is

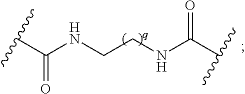

q is 1, 2, 3, 4, 5, 6, 7, 8, 9, or 10; Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (V) wherein X is

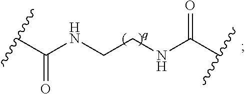

q is 1, 2, 3, 4, 5, or 6; Z¹ is O; Z² is NH; each m is independently selected from 0, 1, 2, 3, 4, 5, and 6; and each k is independently selected from 1, 2, 3, 4, and 5. In some embodiments is a compound of Formula (V) wherein X is

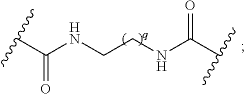

q is 3, 4, or 5; Z¹ is O; Z² is NH; each m is independently selected from 1, 2, 3, 4, and 5; and each k is independently selected from 1, 2, and 3. In some embodiments is a compound of Formula (V) wherein X is

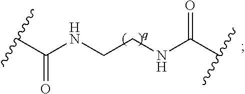

q is 3, 4, or 5; Z¹ is O; Z² is NH; each m is independently selected from 1, 2, 3, 4, and 5; and each k is 1.

In some embodiments is a compound of Formula (V) wherein X is

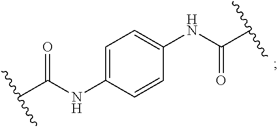 [SMILES: *C(=O)Nc1ccc(NC(*)=O)cc1]

Z¹ is O; Z² is O; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (V) wherein X is

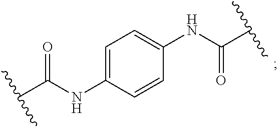 [SMILES: *C(=O)Nc1ccc(NC(*)=O)cc1]

Z¹ is O; Z² is O; each m is independently selected from 0, 1, 2, 3, 4, 5, and 6; and each k is independently selected from 1, 2, 3, 4, and 5. In some embodiments is a compound of Formula (V) wherein X is

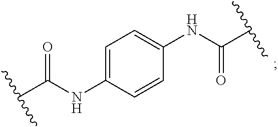 [SMILES: *C(=O)Nc1ccc(NC(*)=O)cc1]

Z¹ is O; Z² is O; each m is independently selected from 1, 2, 3, 4, and 5; and each k is independently selected from 1, 2, and 3. In some embodiments is a compound of Formula (V) wherein X is

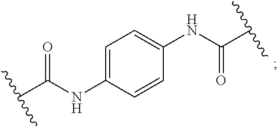 [SMILES: *C(=O)Nc1ccc(NC(*)=O)cc1]

Z¹ is O; Z² is O; each m is independently selected from 1, 2, 3, 4, and 5; and each k is 1.

In some embodiments is a compound of Formula (V) wherein X is

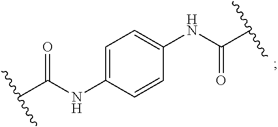 [SMILES: *C(=O)Nc1ccc(NC(*)=O)cc1]

Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (V) wherein X is

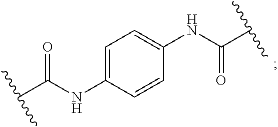 [SMILES: *C(=O)Nc1ccc(NC(*)=O)cc1]

Z¹ is O; Z² is NH; each m is independently selected from 0, 1, 2, 3, 4, 5, and 6; and each k is independently selected from 1, 2, 3, 4, and 5. In some embodiments is a compound of Formula (V) wherein X is

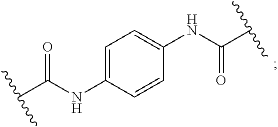 [SMILES: *C(=O)Nc1ccc(NC(*)=O)cc1]

Z¹ is O; Z² is NH; each m is independently selected from 1, 2, 3, 4, and 5; and each k is independently selected from 1, 2, and 3. In some embodiments is a compound of Formula (V) wherein X is

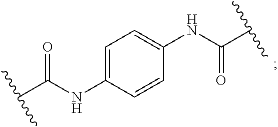 [SMILES: *C(=O)Nc1ccc(NC(*)=O)cc1]

Z¹ is O; Z² is NH; each m is independently selected from 1, 2, 3, 4, and 5; and each k is 1.

In some embodiments is a compound of Formula (V) wherein X is

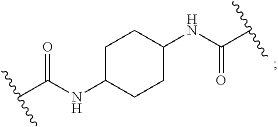 [SMILES: *C(=O)NC1CCC(NC(*)=O)CC1]

Z¹ is O; Z² is O; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (V) wherein X is

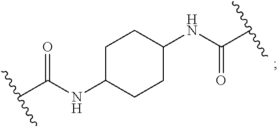 [SMILES: *C(=O)NC1CCC(NC(*)=O)CC1]

Z¹ is O; Z² is O; each m is independently selected from 0, 1, 2, 3, 4, 5, and 6; and each k is independently selected from 1, 2, 3, 4, and 5. In some embodiments is a compound of Formula (V) wherein X is

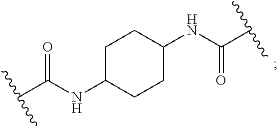 [SMILES: *C(=O)NC1CCC(NC(*)=O)CC1]

Z¹ is O; Z² is O; each m is independently selected from 1, 2, 3, 4, and 5; and each k is independently selected from 1, 2, and 3. In some embodiments is a compound of Formula (V) wherein X is

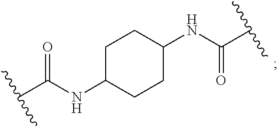 [SMILES: *C(=O)NC1CCC(NC(*)=O)CC1]

Z¹ is O; Z² is O; each m is independently selected from 1, 2, 3, 4, and 5; and each k is 1.

In some embodiments is a compound of Formula (V) wherein X is

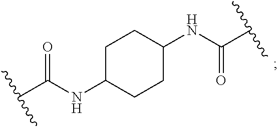 [SMILES: *C(=O)NC1CCC(NC(*)=O)CC1]

Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (V) wherein X is

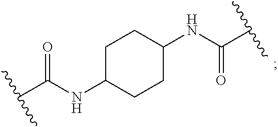 [SMILES: *C(=O)NC1CCC(NC(*)=O)CC1]

Z¹ is O; Z² is NH; each m is independently selected from 0, 1, 2, 3, 4, 5, and 6; and each k is independently selected from 1, 2, 3, 4, and 5. In some embodiments is a compound of Formula (V) wherein X is

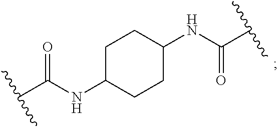 [SMILES: *C(=O)NC1CCC(NC(*)=O)CC1]

Z¹ is O; Z² is NH; each m is independently selected from 1, 2, 3, 4, and 5; and each k is independently selected from 1, 2, and 3. In some embodiments is a compound of Formula (V) wherein X is

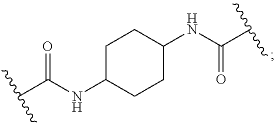 [SMILES: *C(=O)NC1CCC(NC(*)=O)CC1]

Z¹ is O; Z² is NH; each m is independently selected from 1, 2, 3, 4, and 5; and each k is 1.

In some embodiments is a compound of Formula (V) wherein X is alkylene; Z¹ is O; Z² is O; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (V) wherein X is alkylene; Z¹ is O; Z² is O; each m is independently selected from 0, 1, 2, 3, 4, 5, and 6; and each k is independently selected from 1, 2, 3, 4, and 5. In some embodiments is a compound of Formula (V) wherein X is alkylene; Z¹ is O; Z² is O; each m is independently selected from 1, 2, 3, 4, and 5; and each k is independently selected from 1, 2, and 3. In some embodiments is a compound of Formula (V) wherein X is alkylene; Z¹ is O; Z² is O; each m is independently selected from 1, 2, 3, 4, and 5; and each k is 1.

In some embodiments is a compound of Formula (V) wherein X is alkylene; Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (V) wherein X is alkylene; Z¹ is O; Z² is NH; each m is independently selected from 0, 1, 2, 3, 4, 5, and 6; and each k is independently selected from 1, 2, 3, 4, and 5. In some embodiments is a compound of Formula (V) wherein X is alkylene; Z¹ is O; Z² is NH; each m is independently selected from 1, 2, 3, 4, and 5; and each k is independently selected from 1, 2, and 3. In some embodiments is a compound of Formula (V) wherein X is alkylene; Z¹ is O; Z² is NH; each m is independently selected from 1, 2, 3, 4, and 5; and each k is 1.

In some embodiments is a compound of Formula (V) wherein X is —C(O)—; Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (V) wherein X is —C(O)—; Z¹ is O; Z² is NH; each m is independently selected from 0, 1, 2, 3, 4, 5, and 6; and each k is independently selected from 1, 2, 3, 4, and 5. In some embodiments is a compound of Formula (V) wherein X is —C(O)—; Z¹ is O; Z² is NH; each m is independently selected from 1, 2, 3, 4, and 5; and each k is independently selected from 1, 2, and 3. In some embodiments is a compound of Formula (V) wherein X is —C(O)—; Z¹ is O; Z² is NH; each m is independently selected from 1, 2, 3, 4, and 5; and each k is 1.

In some embodiments is a compound of Formula (V) wherein X is —C(O)C(O)—; Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (V) wherein X is —C(O)C(O)—; Z¹ is O; Z² is NH; each m is independently selected from 0, 1, 2, 3, 4, 5, and 6; and each k is independently selected from 1, 2, 3, 4, and 5. In some embodiments is a compound of Formula (V) wherein X is —C(O)C(O)—; Z¹ is O; Z² is NH; each m is independently selected from 1, 2, 3, 4, and 5; and each k is independently selected from 1, 2, and 3. In some embodiments is a compound of Formula (V) wherein X is —C(O)C(O)—; Z¹ is O; Z² is NH; each m is independently selected from 1, 2, 3, 4, and 5; and each k is 1.

In another embodiment, provided herein are compounds of Formula (VI), or pharmaceutically acceptable salts, solvates, polymorphs, prodrugs, metabolites, deuterides, N-oxides, stereoisomers, or isomers thereof:

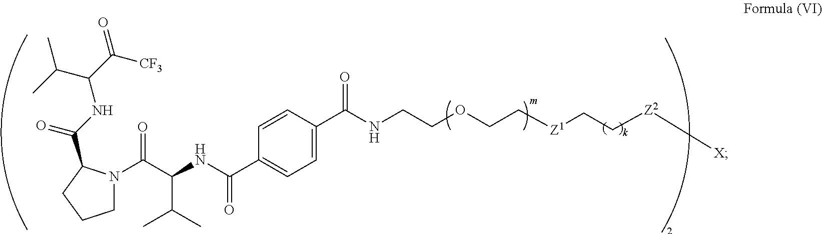

wherein:
X is alkylene, —C(O)—, —C(O)C(O)—,

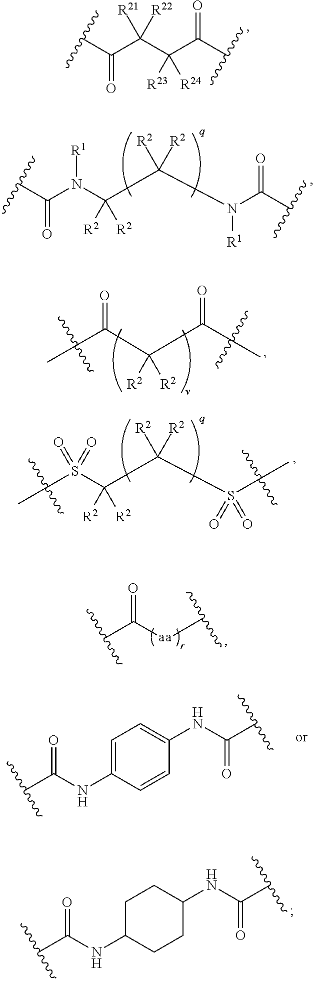

Z¹ is O or CR²R²;
Z² is O or NH;
aa is

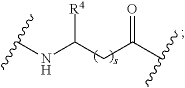

each R¹ is independently selected from H and alkyl;
each R² is independently selected from H, halo, —CN, —SR¹, alkyl, cycloalkyl, haloalkyl, —OR¹, and —R⁴;
each R⁴ is independently selected from alkyl, —CO₂R¹, -(alkylene)-(CO₂R¹), hydroxyalkyl, -(alkylene)(S(O)ₜ)(alkyl), -(alkylene)(NR⁵R⁵), and

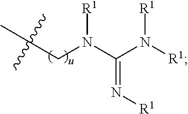

each R⁵ is independently selected from H, alkyl, haloalkyl, arylalkyl, heteroarylalkyl, aryl, and heteroaryl;
R²¹, R²², R²³, and R²⁴ are independently selected from H, halo, —CN, —SR¹, alkyl, cycloalkyl, haloalkyl, —NR¹R¹, and —OR¹;
each k is independently selected from 1, 2, 3, 4, 5, 6, 7, 8, 9, and 10;
each m is independently selected from 0, 1, 2, 3, 4, 5, 6, 7, 8, 9, and 10;
q is 1, 2, 3, 4, 5, 6, 7, 8, 9, or 10;
r is 3, 4, 5, 6, or 7;
each s is independently selected from 0, 1, 2, 3, and 4;
each t is independently selected from 0, 1, and 2;
each u is independently selected from 1, 2, 3, 4, 5, 6, 7, 8, 9, and 10; and
v is selected from 3, 4, 5, 6, 7, 8, 9, and 10.

In some embodiments is a compound of Formula (VI) wherein X is

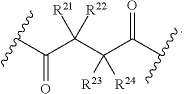

In some embodiments is a compound of Formula (VI) wherein X is

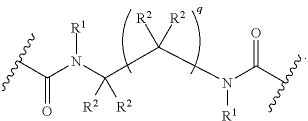

In some embodiments is a compound of Formula (VI) wherein X is

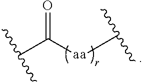

In some embodiments is a compound of Formula (VI) wherein X is

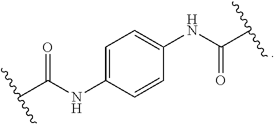 [SMILES: *C(=O)Nc1ccc(NC(*)=O)cc1]

In some embodiments is a compound of Formula (VI) wherein X is

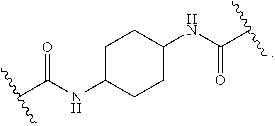 [SMILES: *C(=O)NC1CCC(NC(*)=O)CC1]

In some embodiments is a compound of Formula (VI) wherein X is alkylene. In some embodiments is a compound of Formula (VI) wherein X is —C(O)—. In some embodiments is a compound of Formula (VI) wherein X is —C(O)C(O)—.

In some embodiments is a compound of Formula (VI) wherein X is

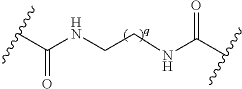

and q is 1. In some embodiments is a compound of Formula (VI) wherein X is

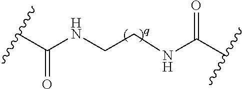

and q is 2. In some embodiments is a compound of Formula (VI) wherein X is

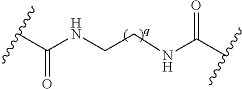

and q is 3. In some embodiments is a compound of Formula (VI) wherein X is

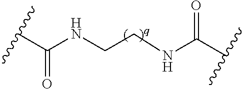

and q is 4. In some embodiments is a compound of Formula (VI) wherein X is

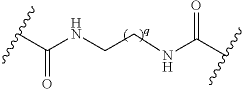

and q is 5. In some embodiments is a compound of Formula (VI) wherein X is

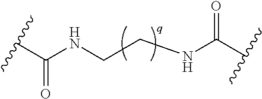

and q is 6. In some embodiments is a compound of Formula (VI) wherein X is

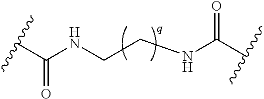

and q is 7.

In some embodiments is a compound of Formula (VI) wherein Z¹ is O; and Z² is O. In some embodiments is a compound of Formula (VI) wherein Z¹ is O; and Z² is NH. In some embodiments is a compound of Formula (VI) wherein Z¹ is CH₂; and Z² is O. In some embodiments is a compound of Formula (VI) wherein Z¹ is CH₂; and Z² is NH.

In some embodiments is a compound of Formula (VI) wherein k is 1. In some embodiments is a compound of Formula (VI) wherein k is 2. In some embodiments is a compound of Formula (VI) wherein k is 3. In some embodiments is a compound of Formula (VI) wherein k is 4. In some embodiments is a compound of Formula (VI) wherein k is 5.

In some embodiments is a compound of Formula (VI) wherein m is 0. In some embodiments is a compound of Formula (VI) wherein m is 1. In some embodiments is a compound of Formula (VI) wherein m is 2. In some embodiments is a compound of Formula (VI) wherein m is 3. In some embodiments is a compound of Formula (VI) wherein m is 4. In some embodiments is a compound of Formula (VI) wherein m is 5. In some embodiments is a compound of Formula (VI) wherein m is 6. In some embodiments is a compound of Formula (VI) wherein m is 7.

In some embodiments is a compound of Formula (VI) wherein X is

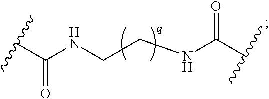

q is 1, 2, 3, 4, 5, 6, 7, 8, 9, or 10; Z¹ is O; Z² is O; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (VI) wherein X is

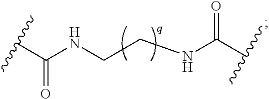

q is 1, 2, 3, 4, 5, or 6; Z¹ is O; Z² is O; each m is independently selected from 0, 1, 2, 3, 4, 5, and 6; and each k is independently selected from 1, 2, 3, 4, and 5. In some embodiments is a compound of Formula (VI) wherein X is

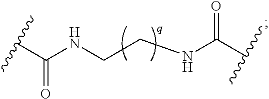

q is 3, 4, or 5; Z¹ is O; Z² is O; each m is independently selected from 1, 2, 3, 4, and 5; and each k is independently selected from 1, 2, and 3. In some embodiments is a compound of Formula (VI) wherein X is

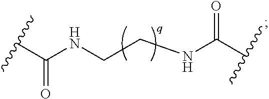

q is 3, 4, or 5; Z¹ is O; Z² is O; each m is independently selected from 1, 2, 3, 4, and 5; and each k is 1.

In some embodiments is a compound of Formula (VI) wherein X is

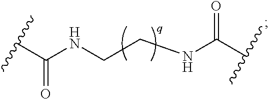

q is 1, 2, 3, 4, 5, 6, 7, 8, 9, or 10; Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (VI) wherein X is

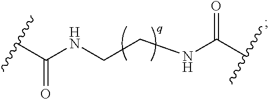

q is 1, 2, 3, 4, 5, or 6; Z¹ is O; Z² is NH; each m is independently selected from 0, 1, 2, 3, 4, 5, and 6; and each k is independently selected from 1, 2, 3, 4, and 5. In some embodiments is a compound of Formula (VI) wherein X is

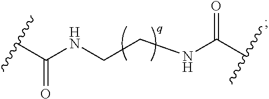

q is 3, 4, or 5; Z¹ is O; Z² is NH; each m is independently selected from 1, 2, 3, 4, and 5; and each k is independently selected from 1, 2, and 3. In some embodiments is a compound of Formula (VI) wherein X is

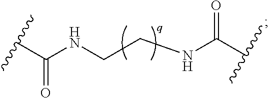

q is 3, 4, or 5; Z¹ is O; Z² is NH; each m is independently selected from 1, 2, 3, 4, and 5; and each k is 1.

In some embodiments is a compound of Formula (VI) wherein X is

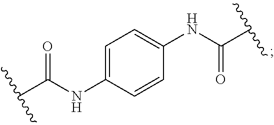 [SMILES: *C(=O)Nc1ccc(NC(*)=O)cc1]

Z¹ is O; Z² is O; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (VI) wherein X is

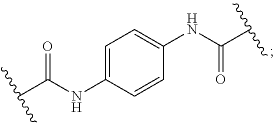 [SMILES: *C(=O)Nc1ccc(NC(*)=O)cc1]

Z¹ is O; Z² is O; each m is independently selected from 0, 1, 2, 3, 4, 5, and 6; and each k is independently selected from 1, 2, 3, 4, and 5. In some embodiments is a compound of Formula (VI) wherein X is

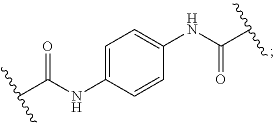 [SMILES: *C(=O)Nc1ccc(NC(*)=O)cc1]

Z¹ is O; Z² is O; each m is independently selected from 1, 2, 3, 4, and 5; and each k is independently selected from 1, 2, and 3. In some embodiments is a compound of Formula (VI) wherein X is

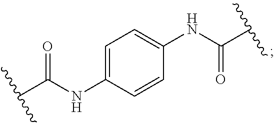 [SMILES: *C(=O)Nc1ccc(NC(*)=O)cc1]

Z¹ is O; Z² is O; each m is independently selected from 1, 2, 3, 4, and 5; and each k is 1.

In some embodiments is a compound of Formula (VI) wherein X is

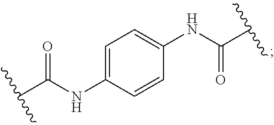 [SMILES: *C(=O)Nc1ccc(NC(*)=O)cc1]

Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (VI) wherein X is

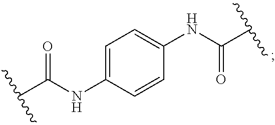 [SMILES: *C(=O)Nc1ccc(NC(*)=O)cc1]

Z¹ is O; Z² is NH; each m is independently selected from 0, 1, 2, 3, 4, 5, and 6; and each k is independently selected from 1, 2, 3, 4, and 5. In some embodiments is a compound of Formula (VI) wherein X is

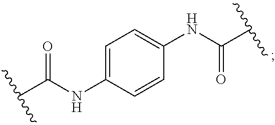 [SMILES: *C(=O)Nc1ccc(NC(*)=O)cc1]

Z¹ is O; Z² is NH; each m is independently selected from 1, 2, 3, 4, and 5; and each k is independently selected from 1, 2, and 3. In some embodiments is a compound of Formula (VI) wherein X is

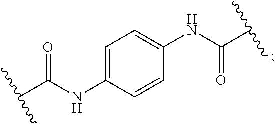 [SMILES: *C(=O)Nc1ccc(NC(*)=O)cc1]

Z¹ is O; Z² is NH; each m is independently selected from 1, 2, 3, 4, and 5; and each k is 1.

In some embodiments is a compound of Formula (VI) wherein X is

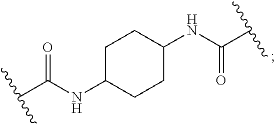 [SMILES: *C(=O)NC1CCC(NC(*)=O)CC1]

Z¹ is O; Z² is O; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (VI) wherein X is

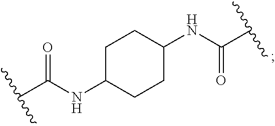 [SMILES: *C(=O)NC1CCC(NC(*)=O)CC1]

Z¹ is O; Z² is O; each m is independently selected from 0, 1, 2, 3, 4, 5, and 6; and each k is independently selected from 1, 2, 3, 4, and 5. In some embodiments is a compound of Formula (VI) wherein X is

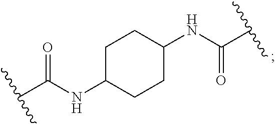 [SMILES: *C(=O)NC1CCC(NC(*)=O)CC1]

Z¹ is O; Z² is O; each m is independently selected from 1, 2, 3, 4, and 5; and each k is independently selected from 1, 2, and 3. In some embodiments is a compound of Formula (VI) wherein X is

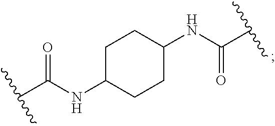 [SMILES: *C(=O)NC1CCC(NC(*)=O)CC1]

Z¹ is O; Z² is O; each m is independently selected from 1, 2, 3, 4, and 5; and each k is 1.

In some embodiments is a compound of Formula (VI) wherein X is

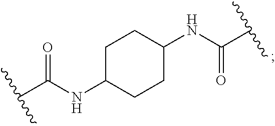 [SMILES: *C(=O)NC1CCC(NC(*)=O)CC1]

Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (VI) wherein X is

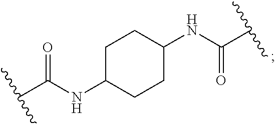 [SMILES: *C(=O)NC1CCC(NC(*)=O)CC1]

Z¹ is O; Z² is NH; each m is independently selected from 0, 1, 2, 3, 4, 5, and 6; and each k is independently selected from 1, 2, 3, 4, and 5. In some embodiments is a compound of Formula (VI) wherein X is

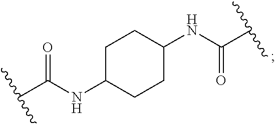 [SMILES: *C(=O)NC1CCC(NC(*)=O)CC1]

Z¹ is O; Z² is NH; each m is independently selected from 1, 2, 3, 4, and 5; and each k is independently selected from 1, 2, and 3. In some embodiments is a compound of Formula (VI) wherein X is

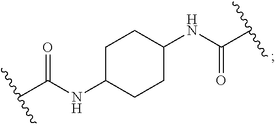 [SMILES: *C(=O)NC1CCC(NC(*)=O)CC1]

Z¹ is O; Z² is NH; each m is independently selected from 1, 2, 3, 4, and 5; and each k is 1.

In some embodiments is a compound of Formula (VI) wherein X is alkylene; Z¹ is O; Z² is O; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (VI) wherein X is alkylene; Z¹ is O; Z² is O; each m is independently selected from 0, 1, 2, 3, 4, 5, and 6; and each k is independently selected from 1, 2, 3, 4, and 5. In some embodiments is a compound of Formula (VI) wherein X is alkylene; Z¹ is O; Z² is O; each m is independently selected from 1, 2, 3, 4, and 5; and each k is independently selected from 1, 2, and 3. In some embodiments is a compound of Formula (VI) wherein X is alkylene; Z¹ is O; Z² is O; each m is independently selected from 1, 2, 3, 4, and 5; and each k is 1.

In some embodiments is a compound of Formula (VI) wherein X is alkylene; Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (VI) wherein X is alkylene; Z¹ is O; Z² is NH; each m is independently selected from 0, 1, 2, 3, 4, 5, and 6; and each k is independently selected from 1, 2, 3, 4, and 5. In some embodiments is a compound of Formula (VI) wherein X is alkylene; Z¹ is O; Z² is NH; each m is independently selected from 1, 2, 3, 4, and 5; and each k is independently selected from 1, 2, and 3. In some embodiments is a compound of Formula (VI) wherein X is alkylene; Z¹ is O; Z² is NH; each m is independently selected from 1, 2, 3, 4, and 5; and each k is 1.

In some embodiments is a compound of Formula (VI) wherein X is —C(O)—; Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (VI) wherein X is —C(O)—; Z¹ is O; Z² is NH; each m is independently selected from 0, 1, 2, 3, 4, 5, and 6; and each k is independently selected from 1, 2, 3, 4, and 5. In some embodiments is a compound of Formula (VI) wherein X is —C(O)—; Z¹ is O; Z² is NH; each m is independently selected from 1, 2, 3, 4, and 5; and each k is independently selected from 1, 2, and 3. In some embodiments is a compound of Formula (VI) wherein X is —C(O)—; Z¹ is O; Z² is NH; each m is independently selected from 1, 2, 3, 4, and 5; and each k is 1.

In some embodiments is a compound of Formula (VI) wherein X is —C(O)C(O)—; Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (VI) wherein X is —C(O)C(O)—; Z¹ is O; Z² is NH; each m is independently selected from 0, 1, 2, 3, 4, 5, and 6; and each k is independently selected from 1, 2, 3, 4, and 5. In some embodiments is a compound of Formula (VI) wherein X is —C(O)C(O)—; Z¹ is O; Z² is NH; each m is independently selected from 1, 2, 3, 4, and 5; and each k is independently selected from 1, 2, and 3. In some embodiments is a compound of Formula (VI) wherein X is —C(O)C(O)—; Z¹ is O; Z² is NH; each m is independently selected from 1, 2, 3, 4, and 5; and each k is 1.

In another embodiment, provided herein are compounds of Formula (VII), or pharmaceutically acceptable salts, solvates, polymorphs, prodrugs, metabolites, deuterides, N-oxides, stereoisomers, or isomers thereof:

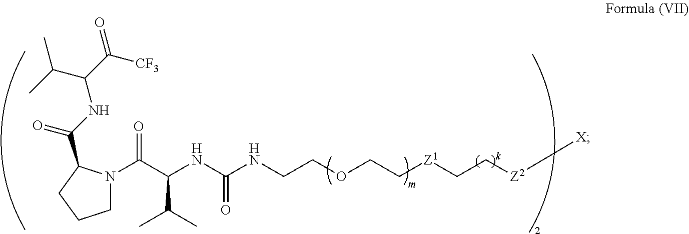

wherein:
X is alkylene, —C(O)—, —C(O)C(O)—,

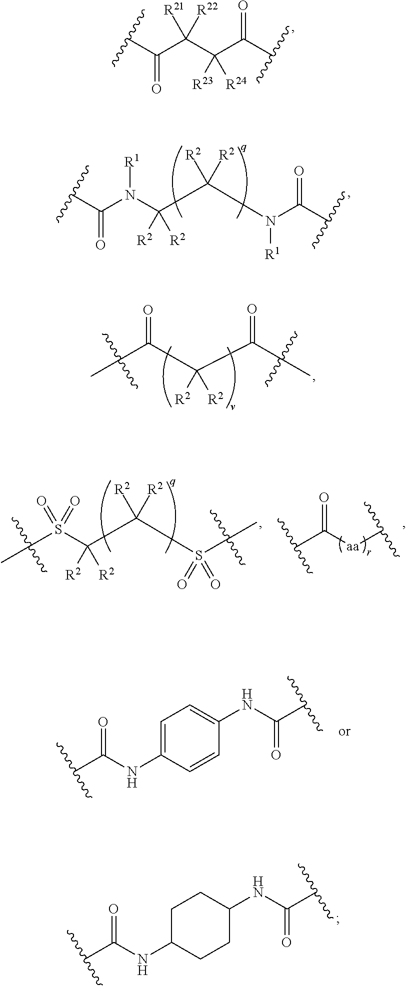

Z¹ is O or CR²R²;
Z² is O or NH;
aa is

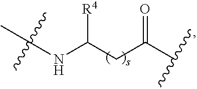

each R¹ is independently selected from H and alkyl;
each R² is independently selected from H, halo, —CN, —SR¹, alkyl, cycloalkyl, haloalkyl, —OR¹, and —R⁴;
each R⁴ is independently selected from alkyl, —CO₂R¹, -(alkylene)-(CO₂R¹), hydroxyalkyl, -(alkylene)(S(O)ₜ)(alkyl), -(alkylene)(NR⁵R⁵), and

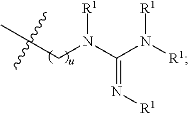

each R⁵ is independently selected from H, alkyl, haloalkyl, arylalkyl, heteroarylalkyl, aryl, and heteroaryl;
R²¹, R²², R²³, and R²⁴ are independently selected from H, halo, —CN, —SR¹, alkyl, cycloalkyl, haloalkyl, —NR¹R¹, and —OR¹;
each k is independently selected from 1, 2, 3, 4, 5, 6, 7, 8, 9, and 10;
each m is independently selected from 0, 1, 2, 3, 4, 5, 6, 7, 8, 9, and 10;
q is 1, 2, 3, 4, 5, 6, 7, 8, 9, or 10;
r is 3, 4, 5, 6, or 7;
each s is independently selected from 0, 1, 2, 3, and 4;
each t is independently selected from 0, 1, and 2;
each u is independently selected from 1, 2, 3, 4, 5, 6, 7, 8, 9, and 10; and
v is selected from 3, 4, 5, 6, 7, 8, 9, and 10.

In some embodiments is a compound of Formula (VII) wherein X is

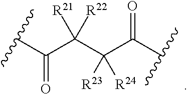

In some embodiments is a compound of Formula (VII) wherein X is

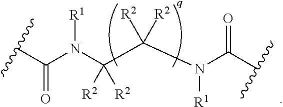

In some embodiments is a compound of Formula (VII) wherein X is

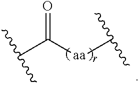

In some embodiments is a compound of Formula (VII) wherein X is

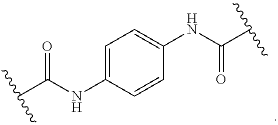 [SMILES: *C(=O)Nc1ccc(NC(*)=O)cc1]

In some embodiments is a compound of Formula (VII) wherein X is

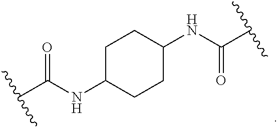 [SMILES: *C(=O)NC1CCC(NC(*)=O)CC1]

In some embodiments is a compound of Formula (VII) wherein X is alkylene. In some embodiments is a compound of Formula (VII) wherein X is —C(O)—. In some embodiments is a compound of Formula (VII) wherein X is —C(O)C(O)—.

In some embodiments is a compound of Formula (VII) wherein X is

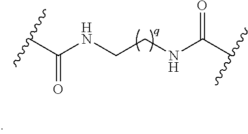

and q is 1. In some embodiments is a compound of Formula (VII) wherein X is

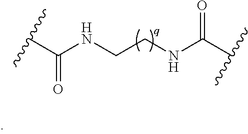

and q is 2. In some embodiments is a compound of Formula (VII) wherein X is

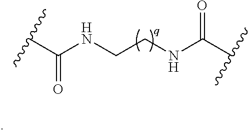

and q is 3. In some embodiments is a compound of Formula (VII) wherein X is

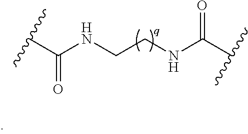

and q is 4. In some embodiments is a compound of Formula (VII) wherein X is

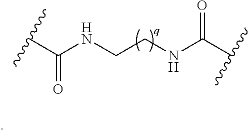

and q is 5. In some embodiments is a compound of Formula (VII) wherein X is

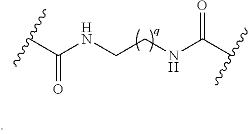

and q is 6. In some embodiments is a compound of Formula (VII) wherein X is

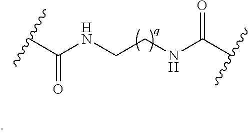

and q is 7.

In some embodiments is a compound of Formula (VII) wherein Z¹ is O; and Z² is O. In some embodiments is a compound of Formula (VII) wherein Z¹ is O; and Z² is NH. In some embodiments is a compound of Formula (VII) wherein Z¹ is CH₂; and Z² is O. In some embodiments is a compound of Formula (VII) wherein Z¹ is CH₂; and Z² is NH.

In some embodiments is a compound of Formula (VII) wherein k is 1. In some embodiments is a compound of Formula (VII) wherein k is 2. In some embodiments is a compound of Formula (VII) wherein k is 3. In some embodiments is a compound of Formula (VII) wherein k is 4. In some embodiments is a compound of Formula (VII) wherein k is 5.

In some embodiments is a compound of Formula (VII) wherein m is 0. In some embodiments is a compound of Formula (VII) wherein m is 1. In some embodiments is a compound of Formula (VII) wherein m is 2. In some embodiments is a compound of Formula (VII) wherein m is 3. In some embodiments is a compound of Formula (VII) wherein m is 4. In some embodiments is a compound of Formula (VII) wherein m is 5. In some embodiments is a compound of Formula (VII) wherein m is 6. In some embodiments is a compound of Formula (VII) wherein m is 7.

In some embodiments is a compound of Formula (VII) wherein X is

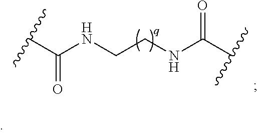

q is 1, 2, 3, 4, 5, 6, 7, 8, 9, or 10; Z¹ is O; Z² is O; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (VII) wherein X is

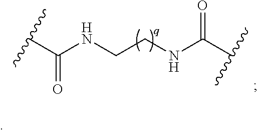

q is 1, 2, 3, 4, 5, or 6; Z¹ is O; Z² is O; each m is independently selected from 0, 1, 2, 3, 4, 5, and 6; and each k is independently selected from 1, 2, 3, 4, and 5. In some embodiments is a compound of Formula (VII) wherein X is

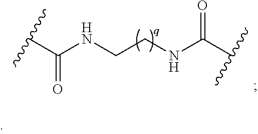

q is 3, 4, or 5; Z¹ is O; Z² is O; each m is independently selected from 1, 2, 3, 4, and 5; and each k is independently selected from 1, 2, and 3. In some embodiments is a compound of Formula (VII) wherein X is

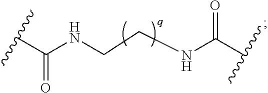

q is 3, 4, or 5; Z¹ is O; Z² is O; each m is independently selected from 1, 2, 3, 4, and 5; and each k is 1.

In some embodiments is a compound of Formula (VII) wherein X is

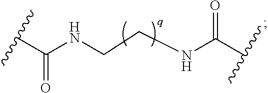

q is 1, 2, 3, 4, 5, 6, 7, 8, 9, or 10; Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (VII) wherein X is

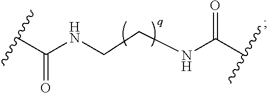

q is 1, 2, 3, 4, 5, or 6; Z¹ is O; Z² is NH; each m is independently selected from 0, 1, 2, 3, 4, 5, and 6; and each k is independently selected from 1, 2, 3, 4, and 5. In some embodiments is a compound of Formula (VII) wherein X is

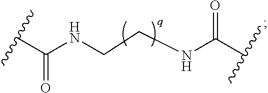

q is 3, 4, or 5; Z¹ is O; Z² is NH; each m is independently selected from 1, 2, 3, 4, and 5; and each k is independently selected from 1, 2, and 3. In some embodiments is a compound of Formula (VII) wherein X is

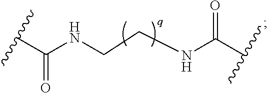

q is 3, 4, or 5; Z¹ is O; Z² is NH; each m is independently selected from 1, 2, 3, 4, and 5; and each k is 1.

In some embodiments is a compound of Formula (VII) wherein X is

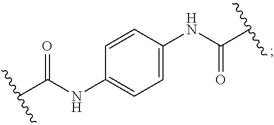 [SMILES: *C(=O)Nc1ccc(NC(*)=O)cc1]

Z¹ is O; Z² is O; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (VII) wherein X is

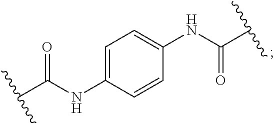 [SMILES: *C(=O)Nc1ccc(NC(*)=O)cc1]

Z¹ is O; Z² is O; each m is independently selected from 0, 1, 2, 3, 4, 5, and 6; and each k is independently selected from 1, 2, 3, 4, and 5. In some embodiments is a compound of Formula (VII) wherein X is

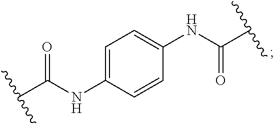 [SMILES: *C(=O)Nc1ccc(NC(*)=O)cc1]

Z¹ is O; Z² is O; each m is independently selected from 1, 2, 3, 4, and 5; and each k is independently selected from 1, 2, and 3. In some embodiments is a compound of Formula (VII) wherein X is

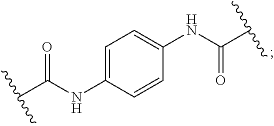 [SMILES: *C(=O)Nc1ccc(NC(*)=O)cc1]

Z¹ is O; Z² is O; each m is independently selected from 1, 2, 3, 4, and 5; and each k is 1.

In some embodiments is a compound of Formula (VII) wherein X is

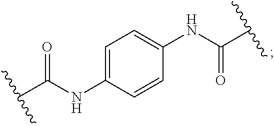 [SMILES: *C(=O)Nc1ccc(NC(*)=O)cc1]

Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (VII) wherein X is

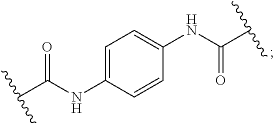 [SMILES: *C(=O)Nc1ccc(NC(*)=O)cc1]

Z¹ is O; Z² is NH; each m is independently selected from 0, 1, 2, 3, 4, 5, and 6; and each k is independently selected from 1, 2, 3, 4, and 5. In some embodiments is a compound of Formula (VII) wherein X is

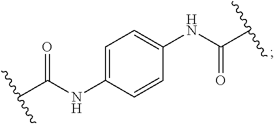 [SMILES: *C(=O)Nc1ccc(NC(*)=O)cc1]

Z¹ is O; Z² is NH; each m is independently selected from 1, 2, 3, 4, and 5; and each k is independently selected from 1, 2, and 3. In some embodiments is a compound of Formula (VII) wherein X is

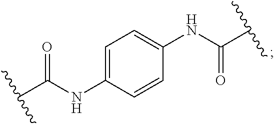 [SMILES: *C(=O)Nc1ccc(NC(*)=O)cc1]

Z¹ is O; Z² is NH; each m is independently selected from 1, 2, 3, 4, and 5; and each k is 1.

In some embodiments is a compound of Formula (VII) wherein X is

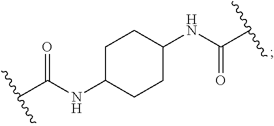 [SMILES: *C(=O)NC1CCC(NC(*)=O)CC1]

Z¹ is O; Z² is O; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (VII) wherein X is

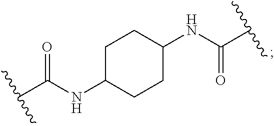 [SMILES: *C(=O)NC1CCC(NC(*)=O)CC1]

Z¹ is O; Z² is O; each m is independently selected from 0, 1, 2, 3, 4, 5, and 6; and each k is independently selected from 1, 2, 3, 4, and 5. In some embodiments is a compound of Formula (VII) wherein X is

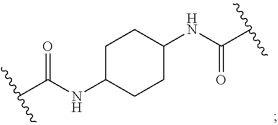 [SMILES: *C(=O)NC1CCC(NC(*)=O)CC1]

Z¹ is O; Z² is O; each m is independently selected from 1, 2, 3, 4, and 5; and each k is independently selected from 1, 2, and 3. In some embodiments is a compound of Formula (VII) wherein X is

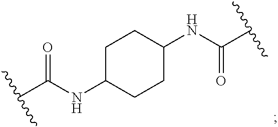 [SMILES: *C(=O)NC1CCC(NC(*)=O)CC1]

Z¹ is O; Z² is O; each m is independently selected from 1, 2, 3, 4, and 5; and each k is 1.

In some embodiments is a compound of Formula (VII) wherein X is

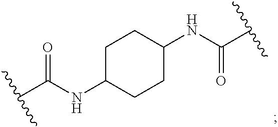 [SMILES: *C(=O)NC1CCC(NC(*)=O)CC1]

Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (VII) wherein X is

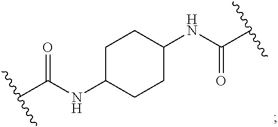 [SMILES: *C(=O)NC1CCC(NC(*)=O)CC1]

Z¹ is O; Z² is NH; each m is independently selected from 0, 1, 2, 3, 4, 5, and 6; and each k is independently selected from 1, 2, 3, 4, and 5. In some embodiments is a compound of Formula (VII) wherein X is

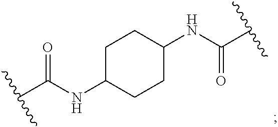 [SMILES: *C(=O)NC1CCC(NC(*)=O)CC1]

Z¹ is O; Z² is NH; each m is independently selected from 1, 2, 3, 4, and 5; and each k is independently selected from 1, 2, and 3. In some embodiments is a compound of Formula (VII) wherein X is

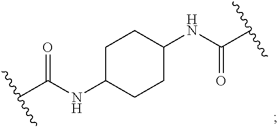 [SMILES: *C(=O)NC1CCC(NC(*)=O)CC1]

Z¹ is O; Z² is NH; each m is independently selected from 1, 2, 3, 4, and 5; and each k is 1.

In some embodiments is a compound of Formula (VII) wherein X is alkylene; Z¹ is O; Z² is O; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (VII) wherein X is alkylene; Z¹ is O; Z² is O; each m is independently selected from 0, 1, 2, 3, 4, 5, and 6; and each k is independently selected from 1, 2, 3, 4, and 5. In some embodiments is a compound of Formula (VII) wherein X is alkylene; Z¹ is O; Z² is O; each m is independently selected from 1, 2, 3, 4, and 5; and each k is independently selected from 1, 2, and 3. In some embodiments is a compound of Formula (VII) wherein X is alkylene; Z¹ is O; Z² is O; each m is independently selected from 1, 2, 3, 4, and 5; and each k is 1.

In some embodiments is a compound of Formula (VII) wherein X is alkylene; Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (VII) wherein X is alkylene; Z¹ is O; Z² is NH; each m is independently selected from 0, 1, 2, 3, 4, 5, and 6; and each k is independently selected from 1, 2, 3, 4, and 5. In some embodiments is a compound of Formula (VII) wherein X is alkylene; Z¹ is O; Z² is NH; each m is independently selected from 1, 2, 3, 4, and 5; and each k is independently selected from 1, 2, and 3. In some embodiments is a compound of Formula (VII) wherein X is alkylene; Z¹ is O; Z² is NH; each m is independently selected from 1, 2, 3, 4, and 5; and each k is 1.

In some embodiments is a compound of Formula (VII) wherein X is —C(O)—; Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (VII) wherein X is —C(O)—; Z¹ is O; Z² is NH; each m is independently selected from 0, 1, 2, 3, 4, 5, and 6; and each k is independently selected from 1, 2, 3, 4, and 5. In some embodiments is a compound of Formula (VII) wherein X is —C(O)—; Z¹ is O; Z² is NH; each m is independently selected from 1, 2, 3, 4, and 5; and each k is independently selected from 1, 2, and 3. In some embodiments is a compound of Formula (VII) wherein X is —C(O)—; Z¹ is O; Z² is NH; each m is independently selected from 1, 2, 3, 4, and 5; and each k is 1.

In some embodiments is a compound of Formula (VII) wherein X is —C(O)C(O)—; Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (VII) wherein X is —C(O)C(O)—; Z¹ is O; Z² is NH; each m is independently selected from 0, 1, 2, 3, 4, 5, and 6; and each k is independently selected from 1, 2, 3, 4, and 5. In some embodiments is a compound of Formula (VII) wherein X is —C(O)C(O)—; Z¹ is O; Z² is NH; each m is independently selected from 1, 2, 3, 4, and 5; and each k is independently selected from 1, 2, and 3. In some embodiments is a compound of Formula (VII) wherein X is —C(O)C(O)—; Z¹ is O; Z² is NH; each m is independently selected from 1, 2, 3, 4, and 5; and each k is 1.

In another embodiment, provided herein are compounds of Formula (VIII), or pharmaceutically acceptable salts, solvates, polymorphs, prodrugs, metabolites, deuterides, N-oxides, stereoisomers, or isomers thereof:

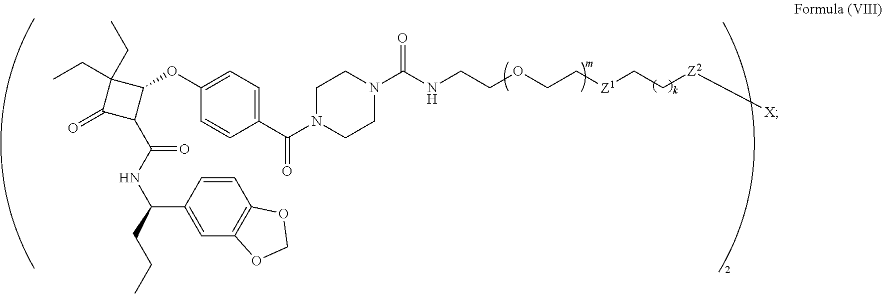

wherein:
X is alkylene, —C(O)—, —C(O)C(O)—,

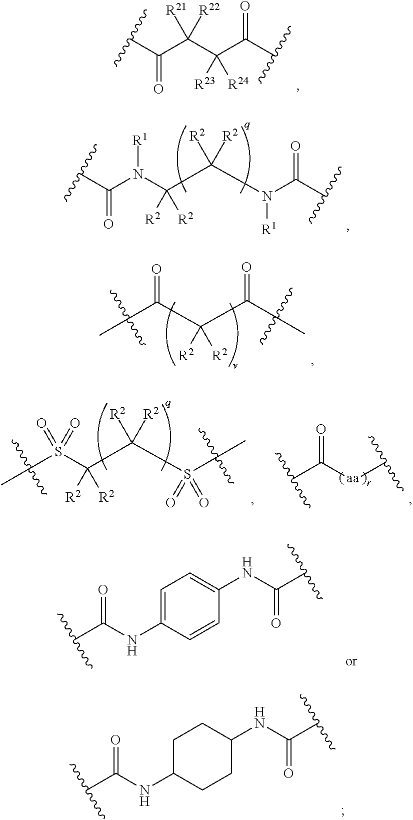

Z¹ is O or CR²R²;
Z² is O or NH;
aa is

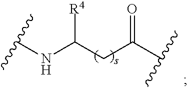

each R¹ is independently selected from H and alkyl;
each R² is independently selected from H, halo, —CN, —SR¹, alkyl, cycloalkyl, haloalkyl, —OR¹, and —R⁴;
each R⁴ is independently selected from alkyl, —CO₂R¹, -(alkylene)-(CO₂R¹), hydroxyalkyl, -(alkylene)(S(O)ₜ)(alkyl), -(alkylene)(NR⁵R⁵), and

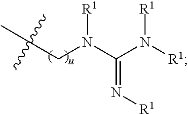

each R⁵ is independently selected from H, alkyl, haloalkyl, arylalkyl, heteroarylalkyl, aryl, and heteroaryl;
R²¹, R²², R²³, and R²⁴ are independently selected from H, halo, —CN, —SR¹, alkyl, cycloalkyl, haloalkyl, —NR¹R¹, and —OR¹;
each k is independently selected from 1, 2, 3, 4, 5, 6, 7, 8, 9, and 10;
each m is independently selected from 0, 1, 2, 3, 4, 5, 6, 7, 8, 9, and 10;
q is 1, 2, 3, 4, 5, 6, 7, 8, 9, or 10;
r is 3, 4, 5, 6, or 7;
each s is independently selected from 0, 1, 2, 3, and 4;
each t is independently selected from 0, 1, and 2;
each u is independently selected from 1, 2, 3, 4, 5, 6, 7, 8, 9, and 10; and
v is selected from 3, 4, 5, 6, 7, 8, 9, and 10.

In some embodiments is a compound of Formula (VIII) wherein X is

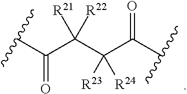

In some embodiments is a compound of Formula (VIII) wherein X is

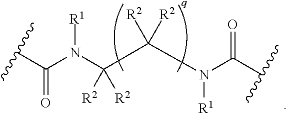

In some embodiments is a compound of Formula (VIII) wherein X is

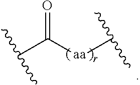

In some embodiments is a compound of Formula (VIII) wherein X is

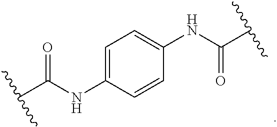 [SMILES: *C(=O)Nc1ccc(NC(*)=O)cc1]

In some embodiments is a compound of Formula (VIII) wherein X is

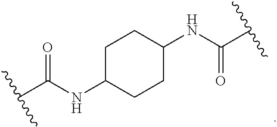 [SMILES: *C(=O)NC1CCC(NC(*)=O)CC1]

In some embodiments is a compound of Formula (VIII) wherein X is alkylene. In some embodiments is a compound of Formula (VIII) wherein X is —C(O)—. In some embodiments is a compound of Formula (VIII) wherein X is —C(O)C(O)—.

In some embodiments is a compound of Formula (VIII) wherein X is

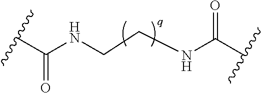

and q is 1. In some embodiments is a compound of Formula (VIII) wherein X is

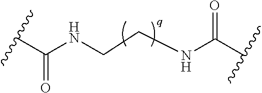

and q is 2. In some embodiments is a compound of Formula (VIII) wherein X is

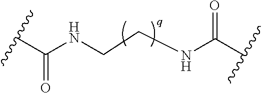

and q is 3. In some embodiments is a compound of Formula (VIII) wherein X is

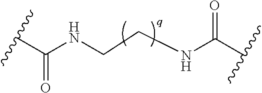

and q is 4. In some embodiments is a compound of Formula (VIII) wherein X is

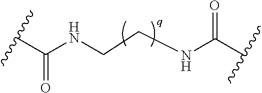

and q is 5. In some embodiments is a compound of Formula (VIII) wherein X is

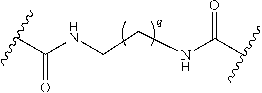

and q is 6. In some embodiments is a compound of Formula (VIII) wherein X is

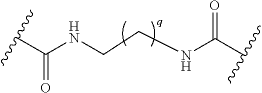

and q is 7.

In some embodiments is a compound of Formula (VIII) wherein Z¹ is O; and Z² is O. In some embodiments is a compound of Formula (VIII) wherein Z¹ is O; and Z² is NH. In some embodiments is a compound of Formula (VIII) wherein Z¹ is CH₂; and Z² is O. In some embodiments is a compound of Formula (VIII) wherein Z¹ is CH₂; and Z² is NH.

In some embodiments is a compound of Formula (VIII) wherein k is 1. In some embodiments is a compound of Formula (VIII) wherein k is 2. In some embodiments is a compound of Formula (VIII) wherein k is 3. In some embodiments is a compound of Formula (VIII) wherein k is 4. In some embodiments is a compound of Formula (VIII) wherein k is 5.

In some embodiments is a compound of Formula (VIII) wherein m is 0. In some embodiments is a compound of Formula (VIII) wherein m is 1. In some embodiments is a compound of Formula (VIII) wherein m is 2. In some embodiments is a compound of Formula (VIII) wherein m is 3. In some embodiments is a compound of Formula (VIII) wherein m is 4. In some embodiments is a compound of Formula (VIII) wherein m is 5. In some embodiments is a compound of Formula (VIII) wherein m is 6. In some embodiments is a compound of Formula (VIII) wherein m is 7.

In some embodiments is a compound of Formula (VIII) wherein X is

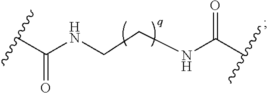

q is 1, 2, 3, 4, 5, 6, 7, 8, 9, or 10; Z¹ is O; Z² is O; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (VIII) wherein X is

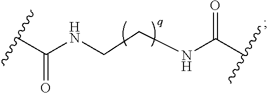

q is 1, 2, 3, 4, 5, or 6; Z¹ is O; Z² is O; each m is independently selected from 0, 1, 2, 3, 4, 5, and 6; and each k is independently selected from 1, 2, 3, 4, and 5. In some embodiments is a compound of Formula (VIII) wherein X is

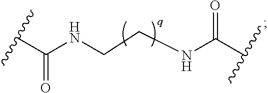

q is 3, 4, or 5; Z¹ is O; Z² is O; each m is independently selected from 1, 2, 3, 4, and 5; and each k is independently selected from 1, 2, and 3. In some embodiments is a compound of Formula (VIII) wherein X is

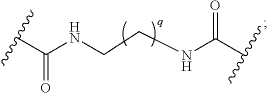

q is 3, 4, or 5; Z¹ is O; Z² is O; each m is independently selected from 1, 2, 3, 4, and 5; and each k is 1.

In some embodiments is a compound of Formula (VIII) wherein X is

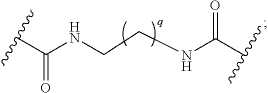

q is 1, 2, 3, 4, 5, 6, 7, 8, 9, or 10; Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (VIII) wherein X is

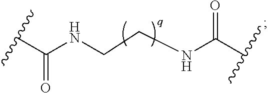

q is 1, 2, 3, 4, 5, or 6; Z¹ is O; Z² is NH; each m is independently selected from 0, 1, 2, 3, 4, 5, and 6; and each k is independently selected from 1, 2, 3, 4, and 5. In some embodiments is a compound of Formula (VIII) wherein X is

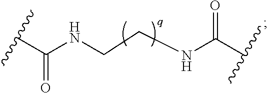

q is 3, 4, or 5; Z¹ is O; Z² is NH; each m is independently selected from 1, 2, 3, 4, and 5; and each k is independently selected from 1, 2, and 3. In some embodiments is a compound of Formula (VIII) wherein X is

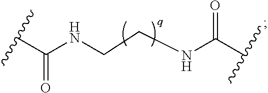

q is 3, 4, or 5; Z¹ is O; Z² is NH; each m is independently selected from 1, 2, 3, 4, and 5; and each k is 1.

In some embodiments is a compound of Formula (VIII) wherein X is

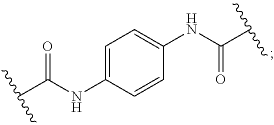 [SMILES: *C(=O)Nc1ccc(NC(*)=O)cc1]

Z¹ is O; Z² is O; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (VIII) wherein X is

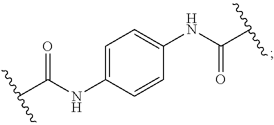 [SMILES: *C(=O)Nc1ccc(NC(*)=O)cc1]

Z¹ is O; Z² is O; each m is independently selected from 0, 1, 2, 3, 4, 5, and 6; and each k is independently selected from 1, 2, 3, 4, and 5. In some embodiments is a compound of Formula (VIII) wherein X is

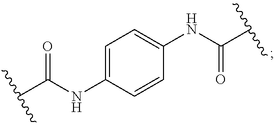 [SMILES: *C(=O)Nc1ccc(NC(*)=O)cc1]

Z¹ is O; Z² is O; each m is independently selected from 1, 2, 3, 4, and 5; and each k is independently selected from 1, 2, and 3. In some embodiments is a compound of Formula (VIII) wherein X is

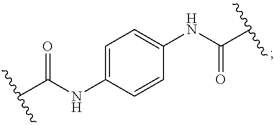 [SMILES: *C(=O)Nc1ccc(NC(*)=O)cc1]

Z¹ is O; Z² is O; each m is independently selected from 1, 2, 3, 4, and 5; and each k is 1.

In some embodiments is a compound of Formula (VIII) wherein X is

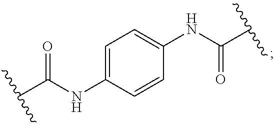 [SMILES: *C(=O)Nc1ccc(NC(*)=O)cc1]

Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (VIII) wherein X is

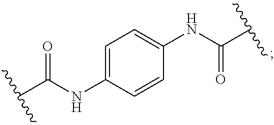 [SMILES: *C(=O)Nc1ccc(NC(*)=O)cc1]

Z¹ is O; Z² is NH; each m is independently selected from 0, 1, 2, 3, 4, 5, and 6; and each k is independently selected from 1, 2, 3, 4, and 5. In some embodiments is a compound of Formula (VIII) wherein X is

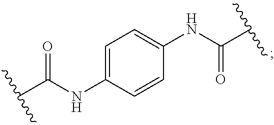 [SMILES: *C(=O)Nc1ccc(NC(*)=O)cc1]

Z¹ is O; Z² is NH; each m is independently selected from 1, 2, 3, 4, and 5; and each k is independently selected from 1, 2, and 3. In some embodiments is a compound of Formula (VIII) wherein X is

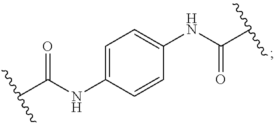 [SMILES: *C(=O)Nc1ccc(NC(*)=O)cc1]

Z¹ is O; Z² is NH; each m is independently selected from 1, 2, 3, 4, and 5; and each k is 1.

In some embodiments is a compound of Formula (VIII) wherein X is

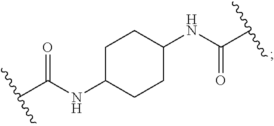 [SMILES: *C(=O)NC1CCC(NC(*)=O)CC1]

Z¹ is O; Z² is O; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (VIII) wherein X is

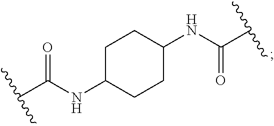 [SMILES: *C(=O)NC1CCC(NC(*)=O)CC1]

Z¹ is O; Z² is O; each m is independently selected from 0, 1, 2, 3, 4, 5, and 6; and each k is independently selected from 1, 2, 3, 4, and 5. In some embodiments is a compound of Formula (VIII) wherein X is

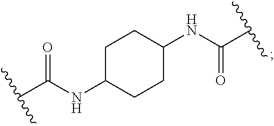 [SMILES: *C(=O)NC1CCC(NC(*)=O)CC1]

Z¹ is O; Z² is O; each m is independently selected from 1, 2, 3, 4, and 5; and each k is independently selected from 1, 2, and 3. In some embodiments is a compound of Formula (VIII) wherein X is

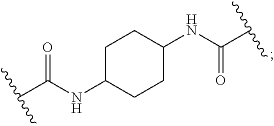 [SMILES: *C(=O)NC1CCC(NC(*)=O)CC1]

Z¹ is O; Z² is O; each m is independently selected from 1, 2, 3, 4, and 5; and each k is 1.

In some embodiments is a compound of Formula (VIII) wherein X is

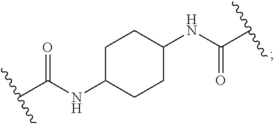 [SMILES: *C(=O)NC1CCC(NC(*)=O)CC1]

Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (VIII) wherein X is

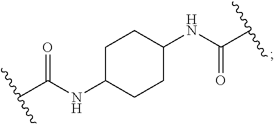 [SMILES: *C(=O)NC1CCC(NC(*)=O)CC1]

Z¹ is O; Z² is NH; each m is independently selected from 0, 1, 2, 3, 4, 5, and 6; and each k is independently selected from 1, 2, 3, 4, and 5. In some embodiments is a compound of Formula (VIII) wherein X is

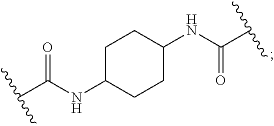 [SMILES: *C(=O)NC1CCC(NC(*)=O)CC1]

Z¹ is O; Z² is NH; each m is independently selected from 1, 2, 3, 4, and 5; and each k is independently selected from 1, 2, and 3. In some embodiments is a compound of Formula (VIII) wherein X is

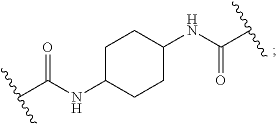 [SMILES: *C(=O)NC1CCC(NC(*)=O)CC1]

Z¹ is O; Z² is NH; each m is independently selected from 1, 2, 3, 4, and 5; and each k is 1.

In some embodiments is a compound of Formula (VIII) wherein X is alkylene; Z¹ is O; Z² is O; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (VIII) wherein X is alkylene; Z¹ is O; Z² is O; each m is independently selected from 0, 1, 2, 3, 4, 5, and 6; and each k is independently selected from 1, 2, 3, 4, and 5. In some embodiments is a compound of Formula (VIII) wherein X is alkylene; Z¹ is O; Z² is O; each m is independently selected from 1, 2, 3, 4, and 5; and each k is independently selected from 1, 2, and 3. In some embodiments is a compound of Formula (VIII) wherein X is alkylene; Z¹ is O; Z² is O; each m is independently selected from 1, 2, 3, 4, and 5; and each k is 1.

In some embodiments is a compound of Formula (VIII) wherein X is alkylene; Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (VIII) wherein X is alkylene; Z¹ is O; Z² is NH; each m is independently selected from 0, 1, 2, 3, 4, 5, and 6; and each k is independently selected from 1, 2, 3, 4, and 5. In some embodiments is a compound of Formula (VIII) wherein X is alkylene; Z¹ is O; Z² is NH; each m is independently selected from 1, 2, 3, 4, and 5; and each k is independently selected from 1, 2, and 3. In some embodiments is a compound of Formula (VIII) wherein X is alkylene; Z¹ is O; Z² is NH; each m is independently selected from 1, 2, 3, 4, and 5; and each k is 1.

In some embodiments is a compound of Formula (VIII) wherein X is —C(O)—; Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (VIII) wherein X is —C(O)—; Z¹ is O; Z² is NH; each m is independently selected from 0, 1, 2, 3, 4, 5, and 6; and each k is independently selected from 1, 2, 3, 4, and 5. In some embodiments is a compound of Formula (VIII) wherein X is —C(O)—; Z¹ is O; Z² is NH; each m is independently selected from 1, 2, 3, 4, and 5; and each k is independently selected from 1, 2, and 3. In some embodiments is a compound of Formula (VIII) wherein X is —C(O)—; Z¹ is O; Z² is NH; each m is independently selected from 1, 2, 3, 4, and 5; and each k is 1.

In some embodiments is a compound of Formula (VIII) wherein X is —C(O)C(O)—; Z¹ is O; Z² is NH; each m is independently selected from 0, 1, 2, 3, 4, 5, 6, 7, 8, 9, and 10; and each k is independently selected from 1, 2, 3, 4, 5, 6, 7, 8, 9, and 10. In some embodiments is a compound of Formula (VIII) wherein X is —C(O)C(O)—; Z¹ is O; Z² is NH; each m is independently selected from 0, 1, 2, 3, 4, 5, and 6; and each k is independently selected from 1, 2, 3, 4, and 5. In some embodiments is a compound of Formula (VIII) wherein X is —C(O)C(O)—; Z¹ is O; Z² is NH; each m is independently selected from 1, 2, 3, 4, and 5; and each k is independently selected from 1, 2, and 3. In some embodiments is a compound of Formula (VIII) wherein X is —C(O)C(O)—; Z¹ is O; Z² is NH; each m is independently selected from 1, 2, 3, 4, and 5; and each k is 1.

In another aspect, provided herein are compounds of Formula (IX), or pharmaceutically acceptable salts, solvates, polymorphs, prodrugs, metabolites, deuterides, N-oxides, stereoisomers, or isomers thereof:

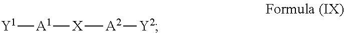

wherein:
A¹ and A² are independently selected from:

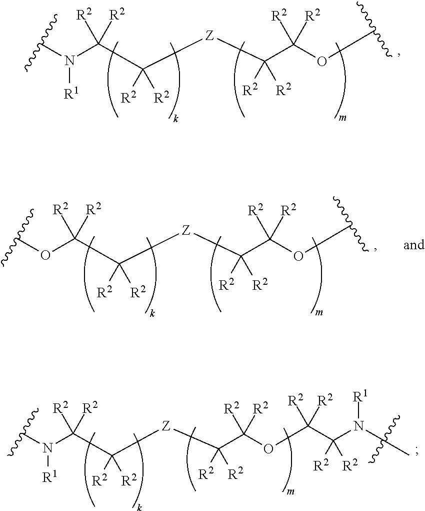

Y¹ and Y² are independently selected from:

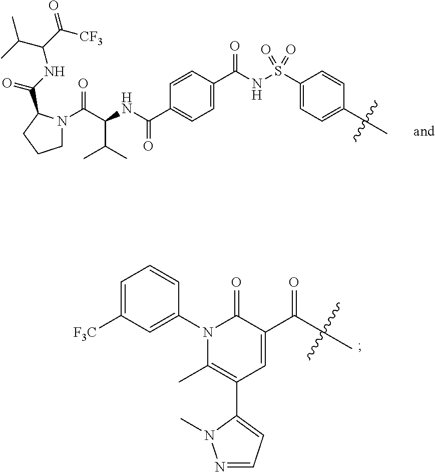 [SMILES: *c1ccc(S(=O)(=O)NC(=O)c2ccc(C(=O)N[C@H](C(=O)N3CCC[C@H]3C(=O)NC(C(=O)C(F)(F)F)C(C)C)C(C)C)cc2)cc1 ; *C(=O)c1cc(-c2ccnn2C)c(C)n(-c2cccc(C(F)(F)F)c2)c1=O]

X is alkylene, —C(O)—,

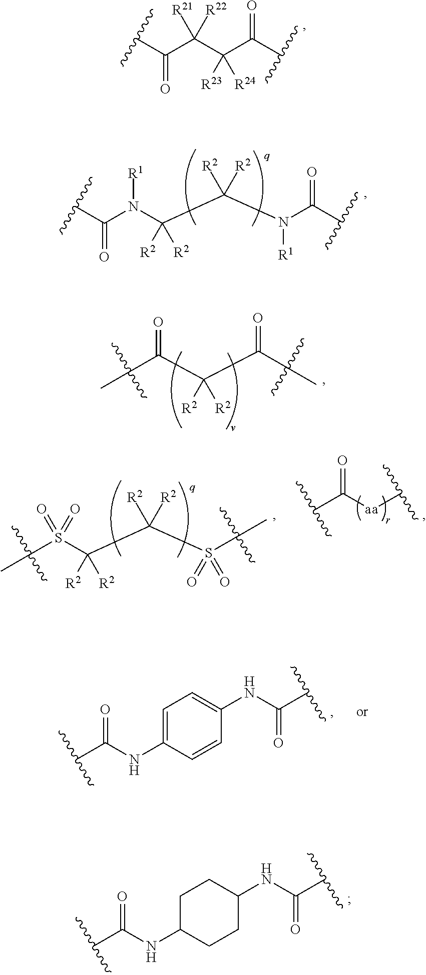

Z is O or CR²R²;
aa is

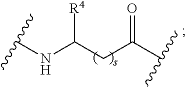

each R¹ is independently selected from H, alkyl, haloalkyl, arylalkyl, and heteroarylalkyl;
each R² is independently selected from H, halo, —CN, —SR¹, alkyl, cycloalkyl, haloalkyl, —OR¹, and —R⁴;
each R⁴ is independently selected from —CO₂R¹, -(alkylene)-(CO₂R¹), hydroxyalkyl, -(alkylene)(S(O)ₜ)(alkyl), -(alkylene)(NR⁵R⁵), and

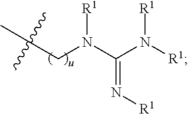

each R⁵ is independently selected from H, alkyl, haloalkyl, arylalkyl, heteroarylalkyl, aryl, and heteroaryl;
R²¹, R²², R²³, and R²⁴ are independently selected from H, halo, —CN, —SR¹, alkyl, cycloalkyl, haloalkyl, —NR¹R¹, and —OR¹;
each k is independently selected from 1, 2, 3, 4, 5, 6, 7, 8, 9, and 10;
each m is independently selected from 0, 1, 2, 3, 4, 5, 6, 7, 8, 9, and 10;
q is 1, 2, 3, 4, 5, 6, 7, 8, 9, or 10;
r is 3, 4, 5, 6, or 7;
each s is independently selected from 0, 1, 2, 3, and 4;
each t is independently selected from 0, 1, and 2;
each u is independently selected from 1, 2, 3, 4, 5, 6, 7, 8, 9, and 10; and
v is selected from 3, 4, 5, 6, 7, 8, 9, and 10.

In some embodiments described above or below of a compound of Formula (IX), Y¹ and Y² are the same. In some embodiments described above or below of a compound of Formula (IX), Y¹ and Y² are

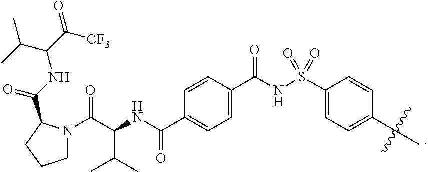 [SMILES: *c1ccc(S(=O)(=O)NC(=O)c2ccc(C(=O)N[C@H](C(=O)N3CCC[C@H]3C(=O)NC(C(=O)C(F)(F)F)C(C)C)C(C)C)cc2)cc1]

In some embodiments described above or below of a compound of Formula (IX), Y¹ and Y² are

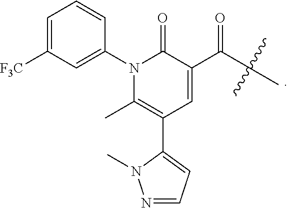 [SMILES: *C(=O)c1cc(-c2ccnn2C)c(C)n(-c2cccc(C(F)(F)F)c2)c1=O]

In some embodiments described above or below of a compound of Formula (IX), A¹ and A² are the same. In some embodiments described above or below of a compound of Formula (IX), A¹ and A² are both

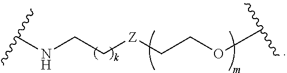

In some embodiments described above or below of a compound of Formula (IX), A¹ and A² are both

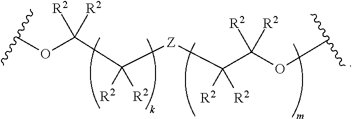

In some embodiments described above or below of a compound of Formula (IX), A¹ and A² are both

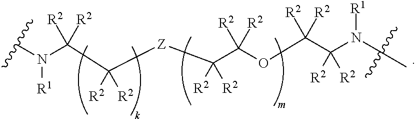

In some embodiments described above or below of a compound of Formula (IX), Z is O. In certain embodiments described above or below of a compound of Formula (IX), each k is independently 1, 2, or 3. In certain embodiments described above or below of a compound of Formula (IX), each k is 1. In certain embodiments described above or below of a compound of Formula (IX), each m is independently 0, 1, 2, or 3. In certain embodiments described above or below of a compound of Formula (IX), each m is 0. In certain embodiments described above or below of a compound of Formula (IX), each m is 1. In certain embodiments described above or below of a compound of Formula (IX), each m is 2. In certain embodiments described above or below of a compound of Formula (IX), each m is 3.

In some embodiments described above or below of a compound of Formula (IX), Z is CR²R². In some embodiments described above or below of a compound of Formula (IX), Z is CH₂.

In some embodiments described above or below of a compound of Formula (IX), L¹ and L² are the same.

In some embodiments described above or below of a compound of Formula (IX), L¹ and L² are both

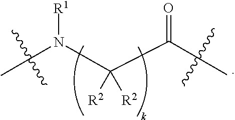

In some embodiments described above or below of a compound of Formula (IX), L¹ and L² are both

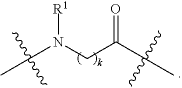

In some embodiments described above or below of a compound of Formula (IX), L¹ and L² are both

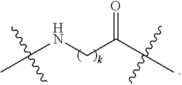

In some embodiments described above or below of a compound of Formula (IX), L¹ and L² are both

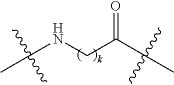

and k is 2. In some embodiments described above or below of a compound of Formula (IX), L¹ and L² are both

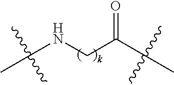

and k is 3. In some embodiments described above or below of a compound of Formula (IX), L¹ and L² are both

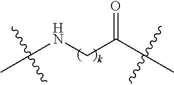

and k is 4. In some embodiments described above or below of a compound of Formula (IX), L¹ and L² are both

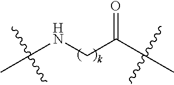

and k is 5.

In some embodiments described above or below of a compound of Formula (IX), X is

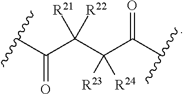

In some embodiments described above or below of a compound of Formula (IX), R²¹ and R²³ are the same and R²² and R²⁴ are the same. In certain embodiments described above or below of a compound of Formula (IX), X is

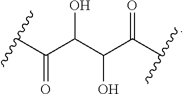 [SMILES: *C(=O)C(O)C(O)C(*)=O]

In certain embodiments described above or below of a compound of Formula (IX), X is

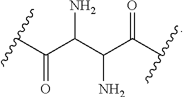 [SMILES: *C(=O)C(N)C(N)C(*)=O]

In some embodiments described above or below of a compound of Formula (IX), R²¹ and R²² are the same. In some embodiments described above or below of a compound of Formula (IX), R²³ and R²⁴ are the same. In some embodiments described above or below of a compound of Formula (IX), R²¹, R²², R²³, and R²⁴ are the same.

In some embodiments described above or below of a compound of Formula (IX), X is

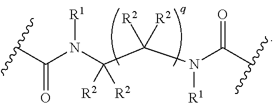

In some embodiments described above or below of a compound of Formula (IX), X is

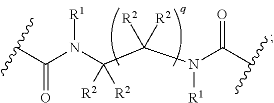

each R² is independently selected from H, alkyl, —OH, —CO₂H, —CO₂alkyl, and -(alkylene)(NH₂); and q is 1, 2, 3, 4, 5, or 6. In certain embodiments described above or below of a compound of Formula (IX), X is

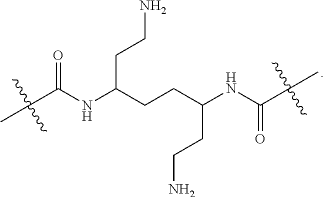 [SMILES: *C(=O)NC(CCN)CCC(CCN)NC(*)=O]

In certain embodiments described above or below of a compound of Formula (IX), X is

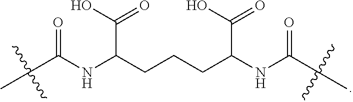 [SMILES: *C(=O)NC(CCCC(NC(*)=O)C(=O)O)C(=O)O]

In certain embodiments described above or below of a compound of Formula (IX), X is

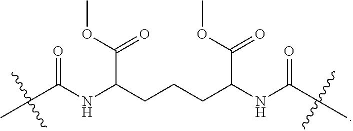 [SMILES: *C(=O)NC(CCCC(NC(*)=O)C(=O)OC)C(=O)OC]

In certain embodiments described above or below of a compound of Formula (IX), X is

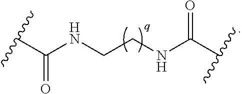

In certain embodiments described above or below of a compound of Formula (IX), q is 1, 2, 3, 4, 5, or 6. In certain embodiments described above or below of a compound of Formula (IX), q is 1. In certain embodiments described above or below of a compound of Formula (IX), q is 2. In certain embodiments described above or below of a compound of Formula (IX), q is 3. In certain embodiments described above or below of a compound of Formula (IX), q is 4. In certain embodiments described above or below of a compound of Formula (IX), q is 5. In certain embodiments described above or below of a compound of Formula (IX), q is 6.

In some embodiments described above or below of a compound of Formula (IX), X is

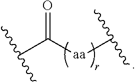

In certain embodiments described above or below of a compound of Formula (IX), aa is selected from:

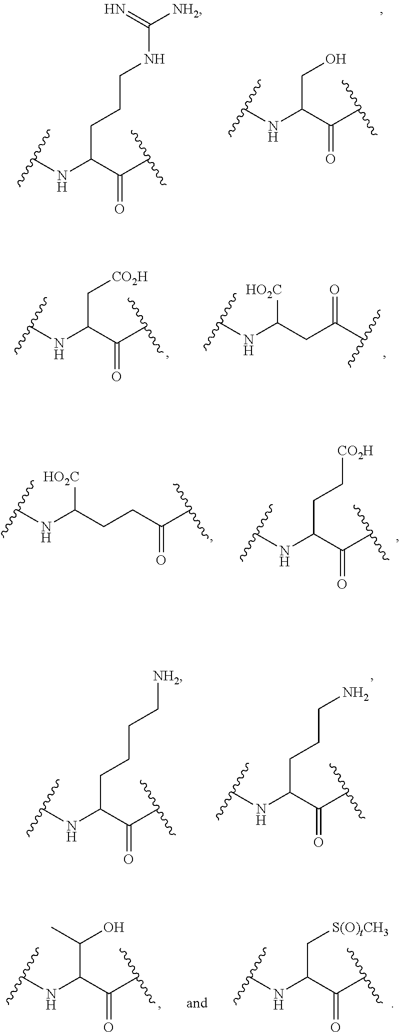

In some embodiments described above or below of a compound of Formula (IX), r is 4, 5, or 6.

In some embodiments described above or below of a compound of Formula (IX), X is

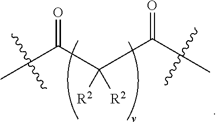

In some embodiments described above or below of a compound of Formula (IX), X is

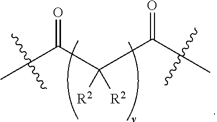

and each R² is independently selected from H, alkyl, —OH, —CO₂H, —CO₂alkyl, and -(alkylene)(NH₂). In certain embodiments described above or below of a compound of Formula (IX), X is

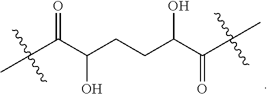 [SMILES: *C(=O)C(O)CCC(O)C(*)=O]

In certain embodiments described above or below of a compound of Formula (IX), X is

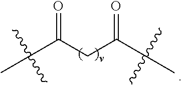

In certain embodiments described above or below of a compound of Formula (IX), X is

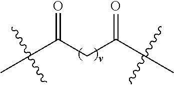

and v is 3. In certain embodiments described above or below of a compound of Formula (IX), X is

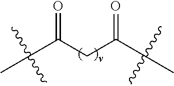

and v is 4. In certain embodiments described above or below of a compound of Formula (IX), X is

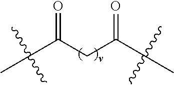

and v is 5. In certain embodiments described above or below of a compound of Formula (IX), X is

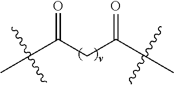

and v is 6.

In some embodiments described above or below of a compound of Formula (IX), X is

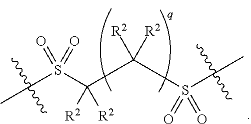

In certain embodiments described above or below of a compound of Formula (IX), X is

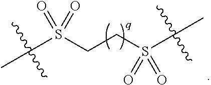

In certain embodiments described above or below of a compound of Formula (IX), X is

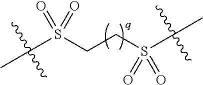

and q is 2. In certain embodiments described above or below of a compound of Formula (IX), X is

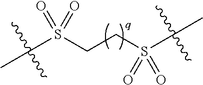

and q is 3. In certain embodiments described above or below of a compound of Formula (IX), X is

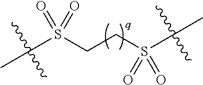

and q is 4.

In some embodiments described above or below of a compound of Formula (IX), X is

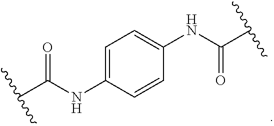 [SMILES: *C(=O)Nc1ccc(NC(*)=O)cc1]

In some embodiments described above or below of a compound of Formula (IX), X is

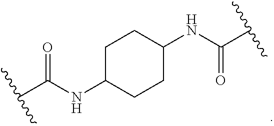 [SMILES: *C(=O)NC1CCC(NC(*)=O)CC1]

In another aspect, provided herein are compounds, or pharmaceutically acceptable salts, solvates, polymorphs, prodrugs, metabolites, N-oxides, stereoisomers, or isomers thereof, selected from:

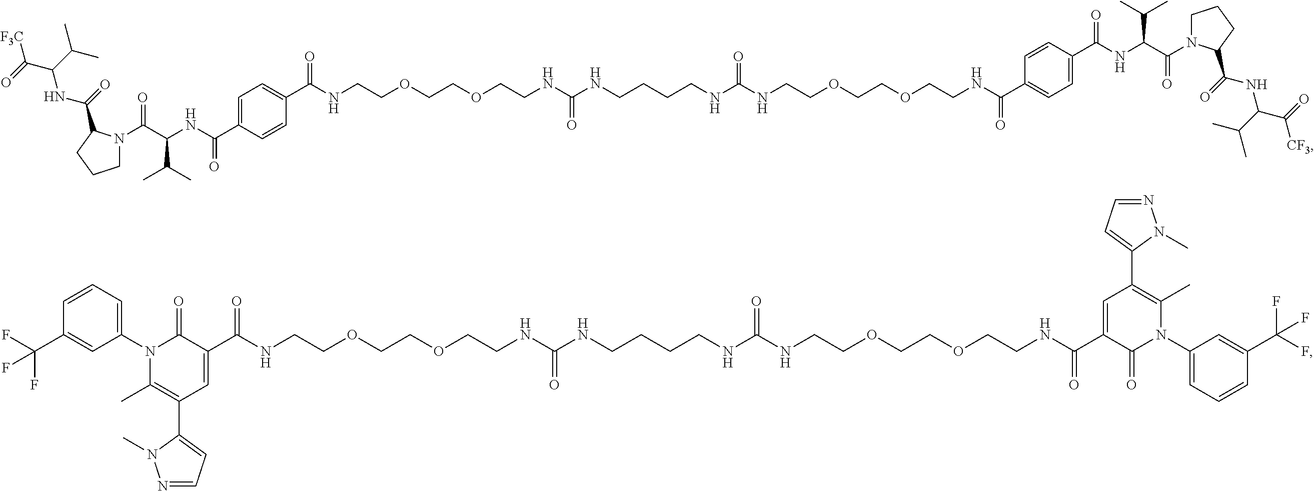

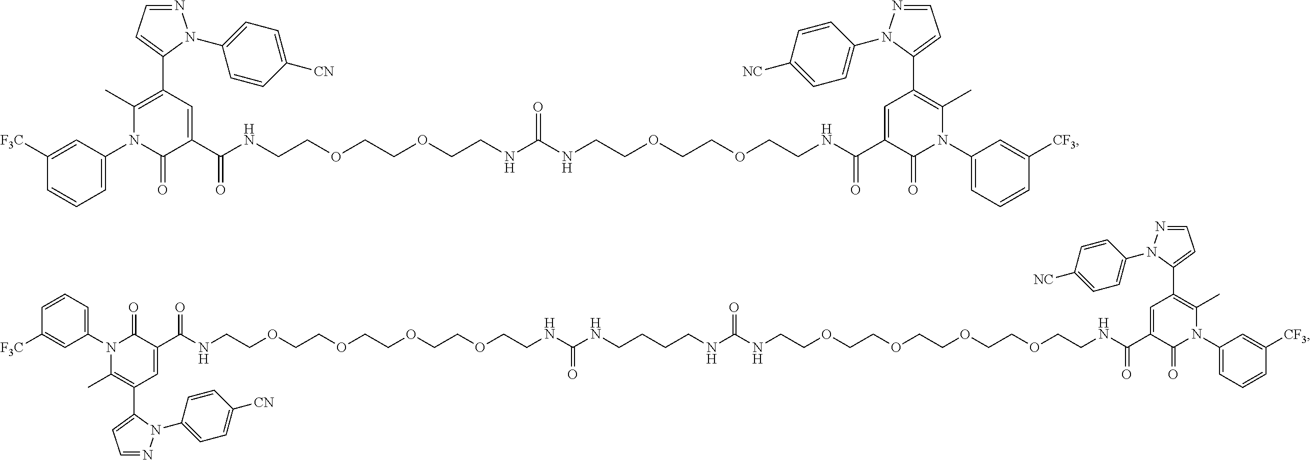 [SMILES: Cc1c(-c2ccnn2-c2ccc(C#N)cc2)cc(C(=O)NCCOCCOCCNC(=O)NCCOCCOCCNC(=O)c2cc(-c3ccnn3-c3ccc(C#N)cc3)c(C)n(-c3cccc(C(F)(F)F)c3)c2=O)c(=O)n1-c1cccc(C(F)(F)F)c1 ; Cc1c(-c2ccnn2-c2ccc(C#N)cc2)cc(C(=O)NCCOCCOCCOCCOCCNC(=O)NCCCCNC(=O)NCCOCCOCCOCCOCCNC(=O)c2cc(-c3ccnn3-c3ccc(C#N)cc3)c(C)n(-c3cccc(C(F)(F)F)c3)c2=O)c(=O)n1-c1cccc(C(F)(F)F)c1]

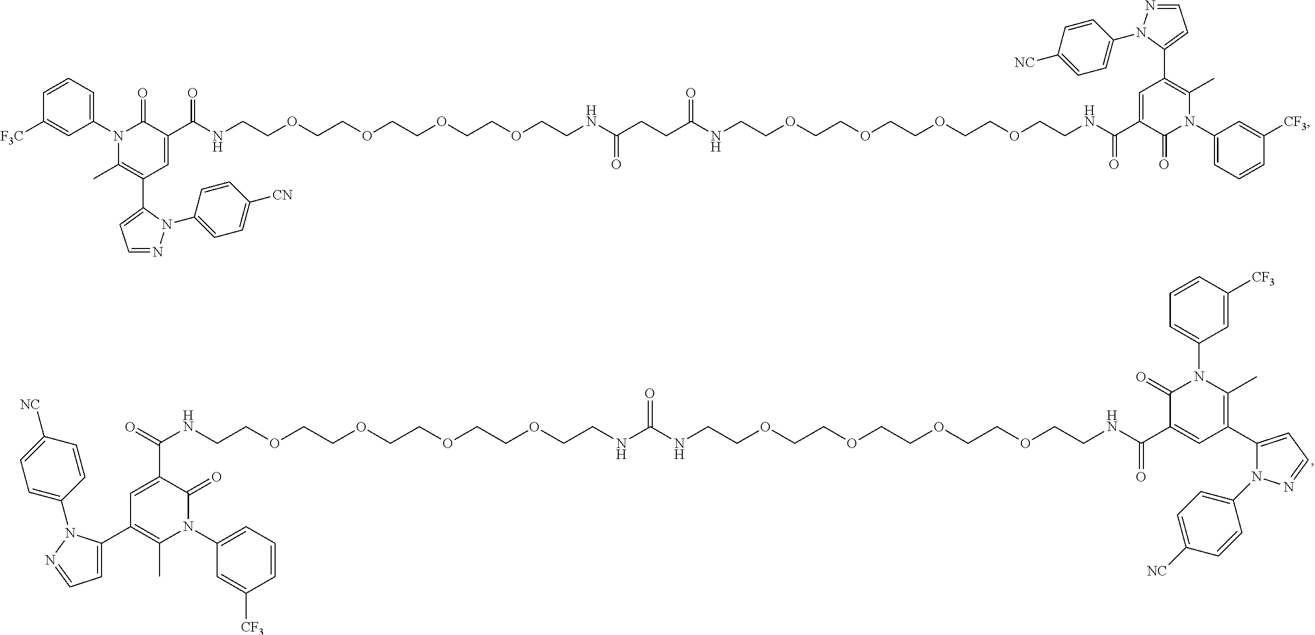 [SMILES: Cc1c(-c2ccnn2-c2ccc(C#N)cc2)cc(C(=O)NCCOCCOCCOCCOCCNC(=O)CCC(=O)NCCOCCOCCOCCOCCNC(=O)c2cc(-c3ccnn3-c3ccc(C#N)cc3)c(C)n(-c3cccc(C(F)(F)F)c3)c2=O)c(=O)n1-c1cccc(C(F)(F)F)c1 ; Cc1c(-c2ccnn2-c2ccc(C#N)cc2)cc(C(=O)NCCOCCOCCOCCOCCNC(=O)NCCOCCOCCOCCOCCNC(=O)c2cc(-c3ccnn3-c3ccc(C#N)cc3)c(C)n(-c3cccc(C(F)(F)F)c3)c2=O)c(=O)n1-c1cccc(C(F)(F)F)c1]

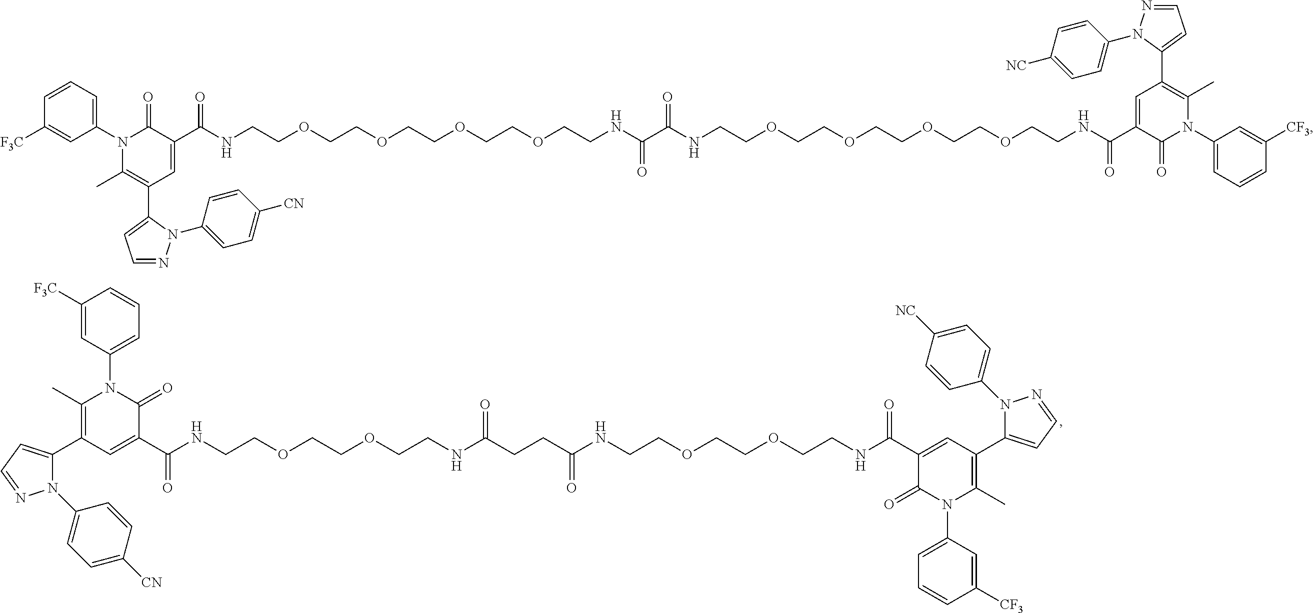

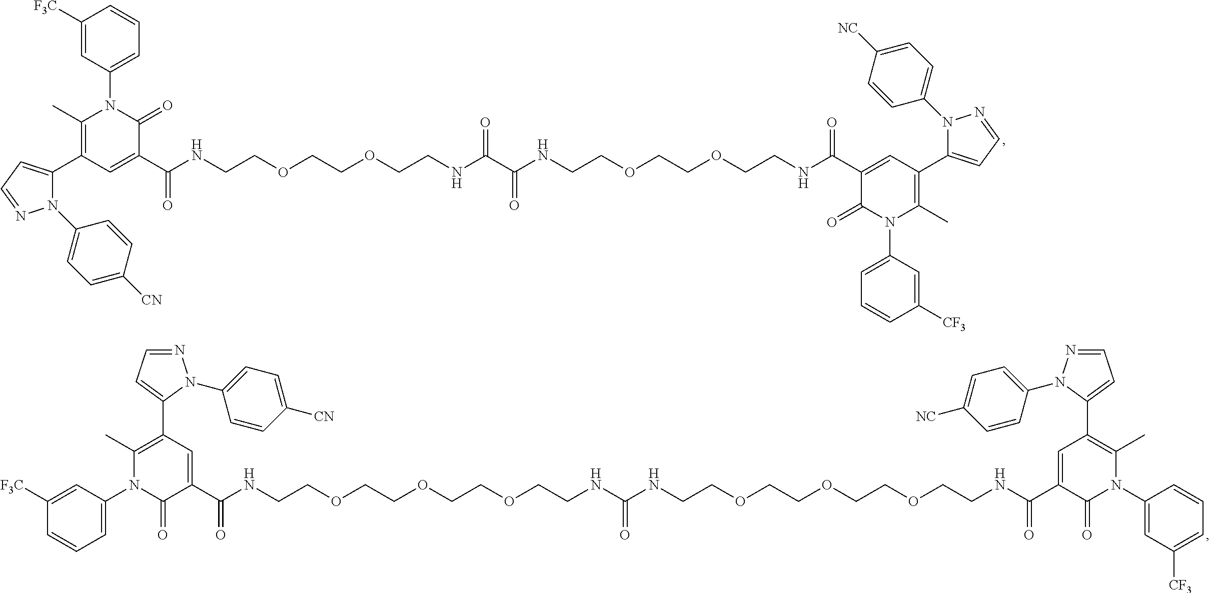 [SMILES: Cc1c(-c2ccnn2-c2ccc(C#N)cc2)cc(C(=O)NCCOCCOCCNC(=O)C(=O)NCCOCCOCCNC(=O)c2cc(-c3ccnn3-c3ccc(C#N)cc3)c(C)n(-c3cccc(C(F)(F)F)c3)c2=O)c(=O)n1-c1cccc(C(F)(F)F)c1 ; Cc1c(-c2ccnn2-c2ccc(C#N)cc2)cc(C(=O)NCCOCCOCCOCCNC(=O)NCCOCCOCCOCCNC(=O)c2cc(-c3ccnn3-c3ccc(C#N)cc3)c(C)n(-c3cccc(C(F)(F)F)c3)c2=O)c(=O)n1-c1cccc(C(F)(F)F)c1]

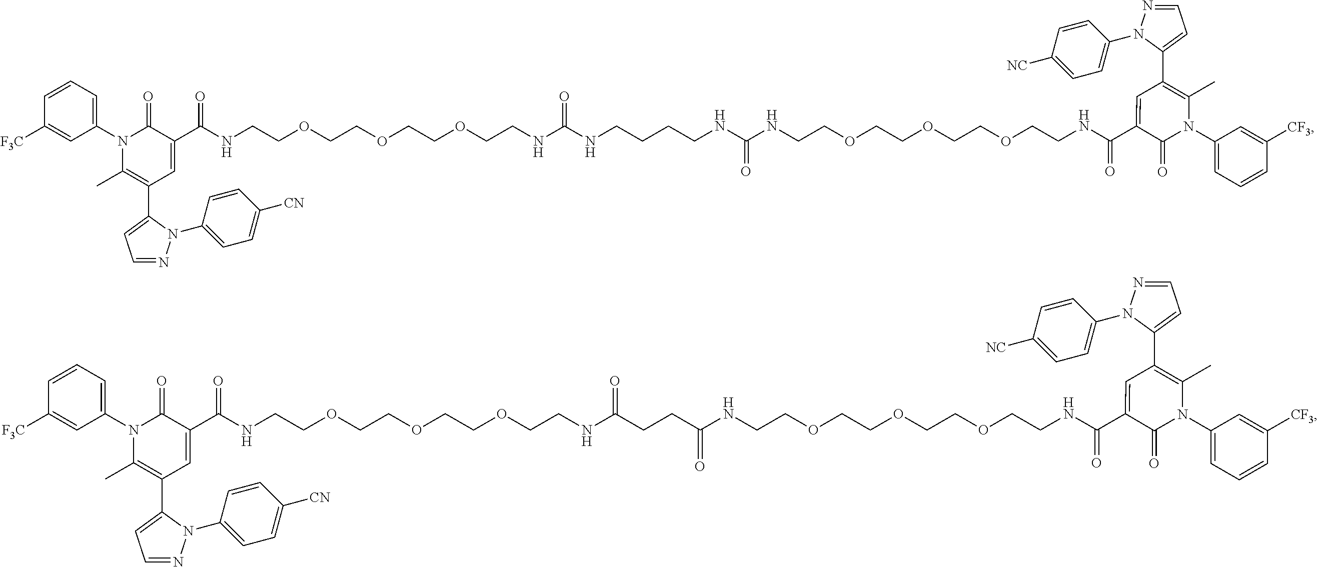 [SMILES: Cc1c(-c2ccnn2-c2ccc(C#N)cc2)cc(C(=O)NCCOCCOCCOCCNC(=O)NCCCCNC(=O)NCCOCCOCCOCCNC(=O)c2cc(-c3ccnn3-c3ccc(C#N)cc3)c(C)n(-c3cccc(C(F)(F)F)c3)c2=O)c(=O)n1-c1cccc(C(F)(F)F)c1 ; Cc1c(-c2ccnn2-c2ccc(C#N)cc2)cc(C(=O)NCCOCCOCCOCCNC(=O)CCC(=O)NCCOCCOCCOCCNC(=O)c2cc(-c3ccnn3-c3ccc(C#N)cc3)c(C)n(-c3cccc(C(F)(F)F)c3)c2=O)c(=O)n1-c1cccc(C(F)(F)F)c1]

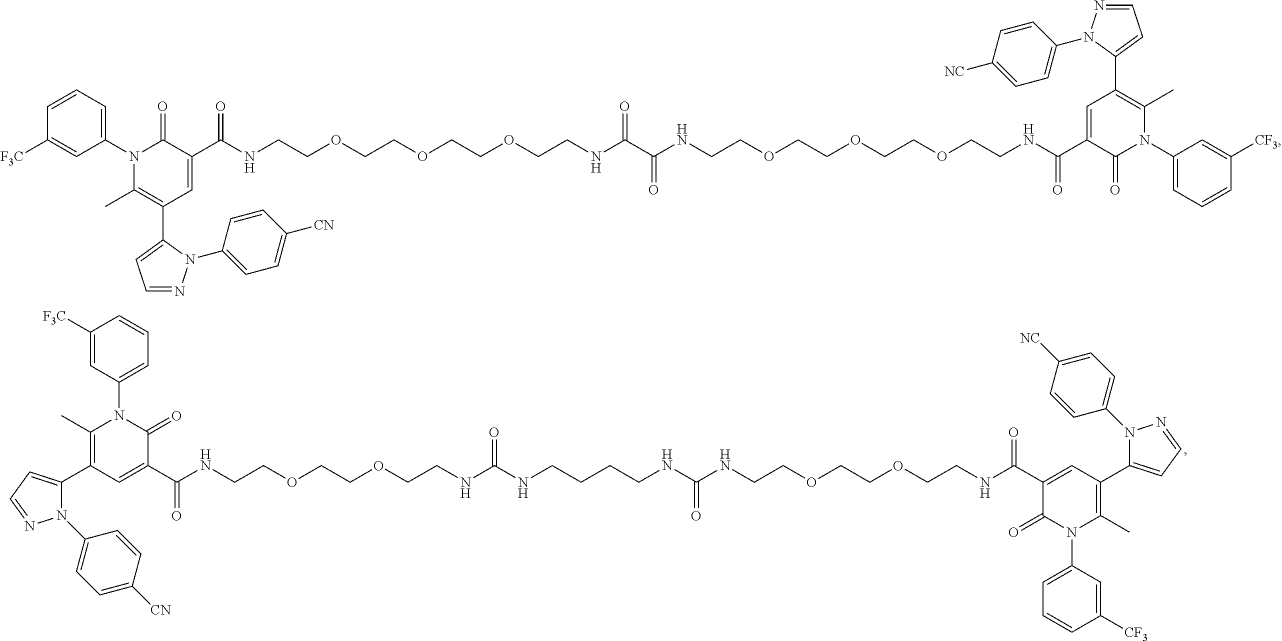

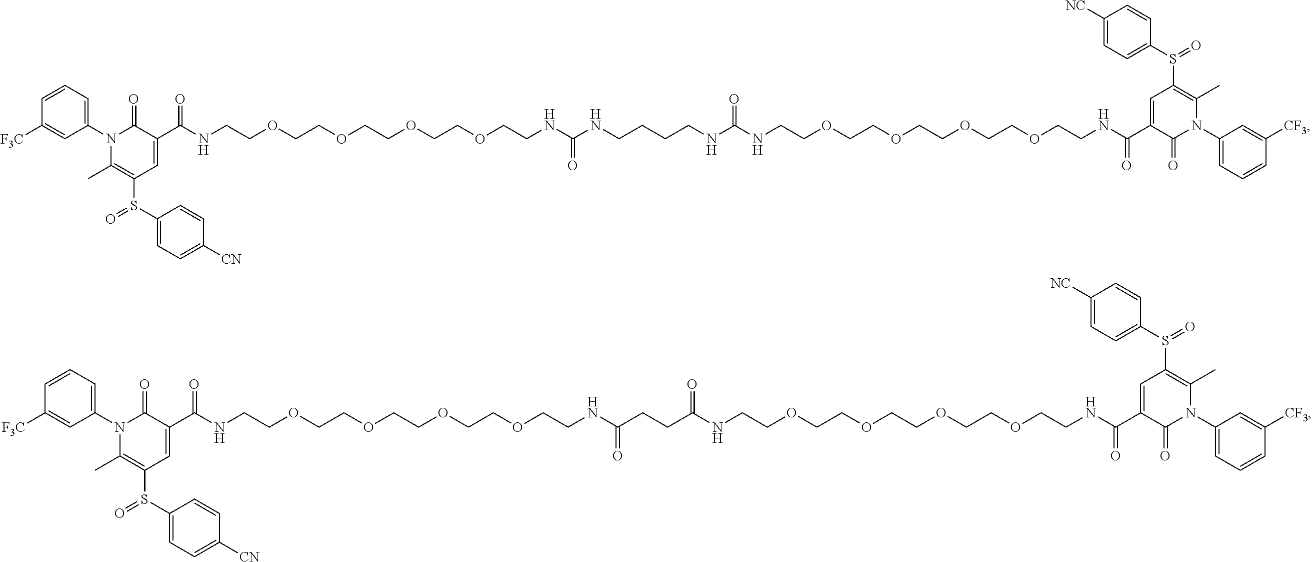 [SMILES: Cc1c(S(=O)c2ccc(C#N)cc2)cc(C(=O)NCCOCCOCCOCCOCCNC(=O)NCCCCNC(=O)NCCOCCOCCOCCOCCNC(=O)c2cc(S(=O)c3ccc(C#N)cc3)c(C)n(-c3cccc(C(F)(F)F)c3)c2=O)c(=O)n1-c1cccc(C(F)(F)F)c1 ; Cc1c(S(=O)c2ccc(C#N)cc2)cc(C(=O)NCCOCCOCCOCCOCCNC(=O)CCC(=O)NCCOCCOCCOCCOCCNC(=O)c2cc(S(=O)c3ccc(C#N)cc3)c(C)n(-c3cccc(C(F)(F)F)c3)c2=O)c(=O)n1-c1cccc(C(F)(F)F)c1]

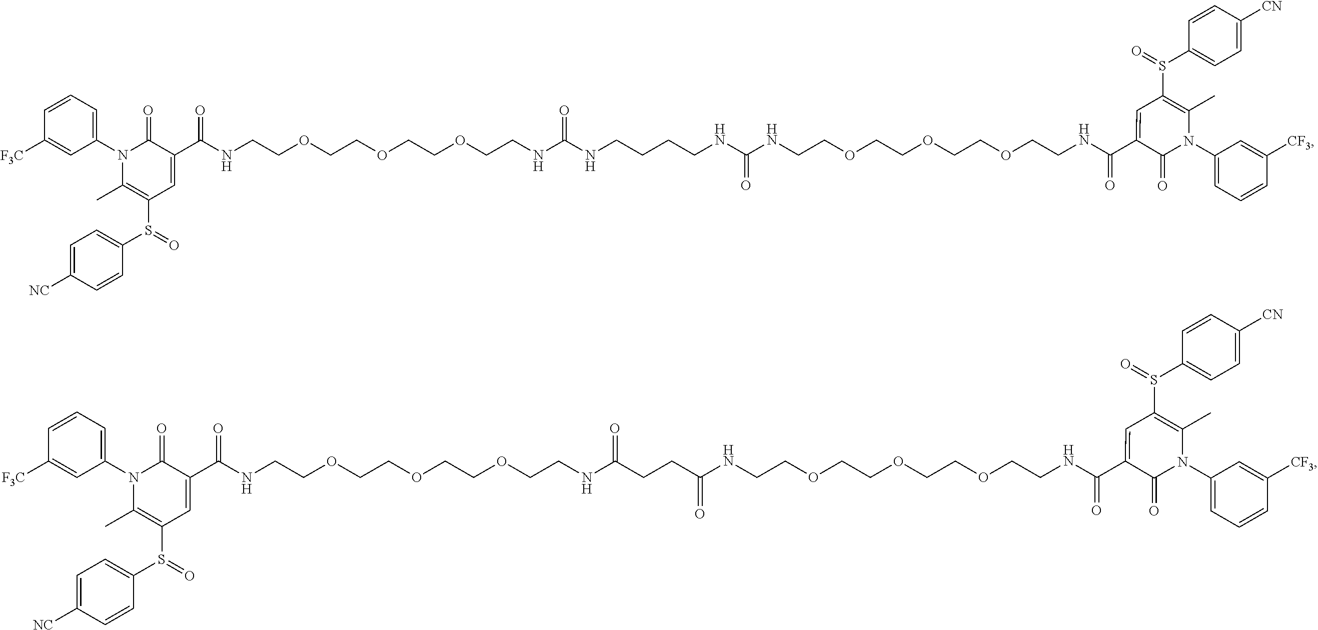 [SMILES: Cc1c(S(=O)c2ccc(C#N)cc2)cc(C(=O)NCCOCCOCCOCCNC(=O)NCCCCNC(=O)NCCOCCOCCOCCNC(=O)c2cc(S(=O)c3ccc(C#N)cc3)c(C)n(-c3cccc(C(F)(F)F)c3)c2=O)c(=O)n1-c1cccc(C(F)(F)F)c1 ; Cc1c(S(=O)c2ccc(C#N)cc2)cc(C(=O)NCCOCCOCCOCCNC(=O)CCC(=O)NCCOCCOCCOCCNC(=O)c2cc(S(=O)c3ccc(C#N)cc3)c(C)n(-c3cccc(C(F)(F)F)c3)c2=O)c(=O)n1-c1cccc(C(F)(F)F)c1]

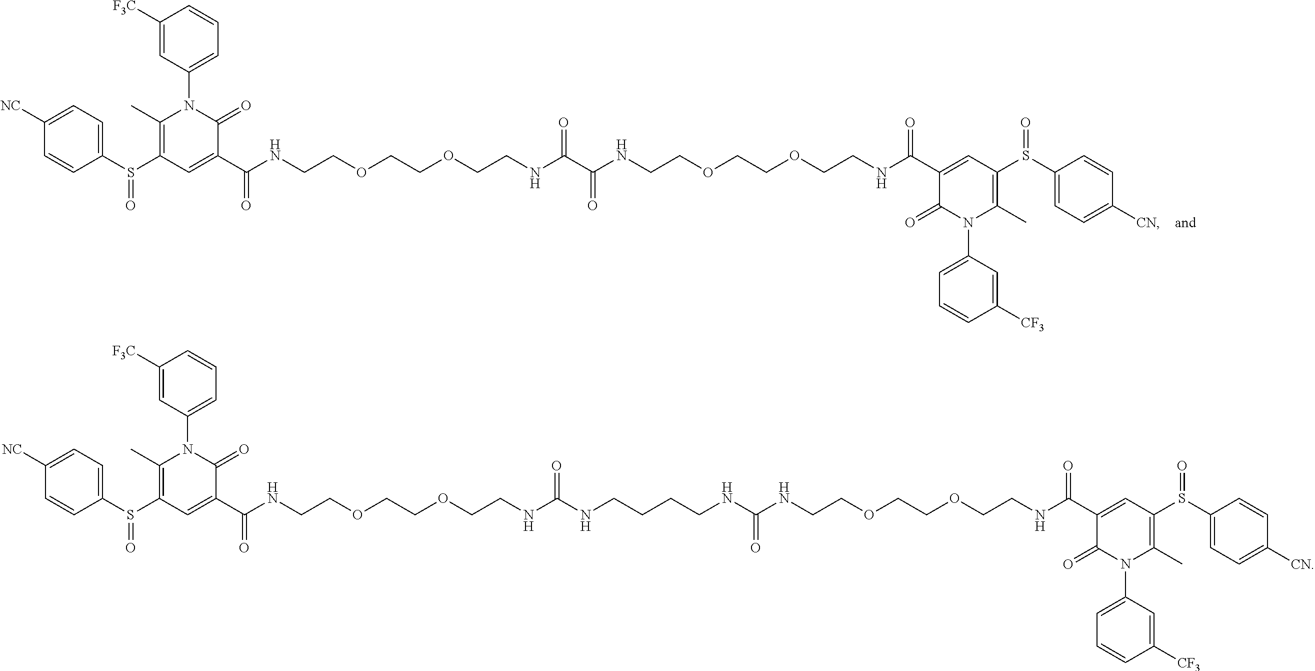 [SMILES: Cc1c(S(=O)c2ccc(C#N)cc2)cc(C(=O)NCCOCCOCCNC(=O)C(=O)NCCOCCOCCNC(=O)c2cc(S(=O)c3ccc(C#N)cc3)c(C)n(-c3cccc(C(F)(F)F)c3)c2=O)c(=O)n1-c1cccc(C(F)(F)F)c1 ; Cc1c(S(=O)c2ccc(C#N)cc2)cc(C(=O)NCCOCCOCCNC(=O)NCCCCNC(=O)NCCOCCOCCNC(=O)c2cc(S(=O)c3ccc(C#N)cc3)c(C)n(-c3cccc(C(F)(F)F)c3)c2=O)c(=O)n1-c1cccc(C(F)(F)F)c1]

In another aspect, provided herein are compounds, or pharmaceutically acceptable salts, solvates, polymorphs, prodrugs, metabolites, N-oxides, stereoisomers, or isomers thereof, selected from:

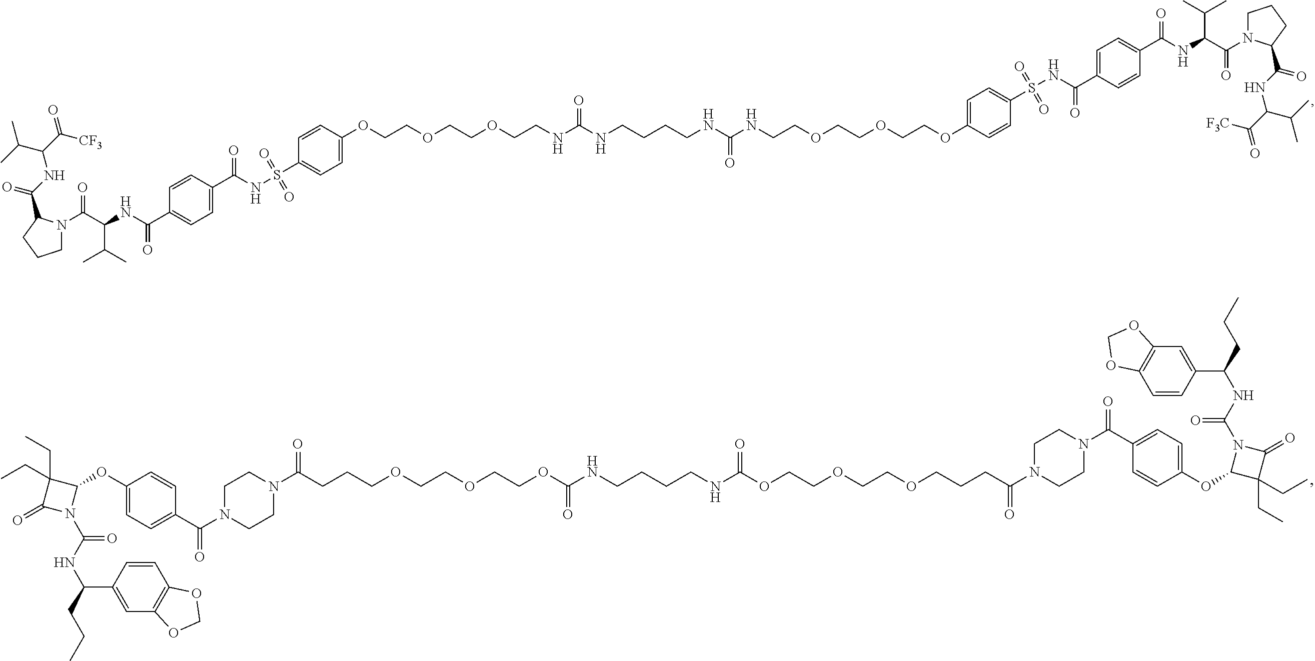

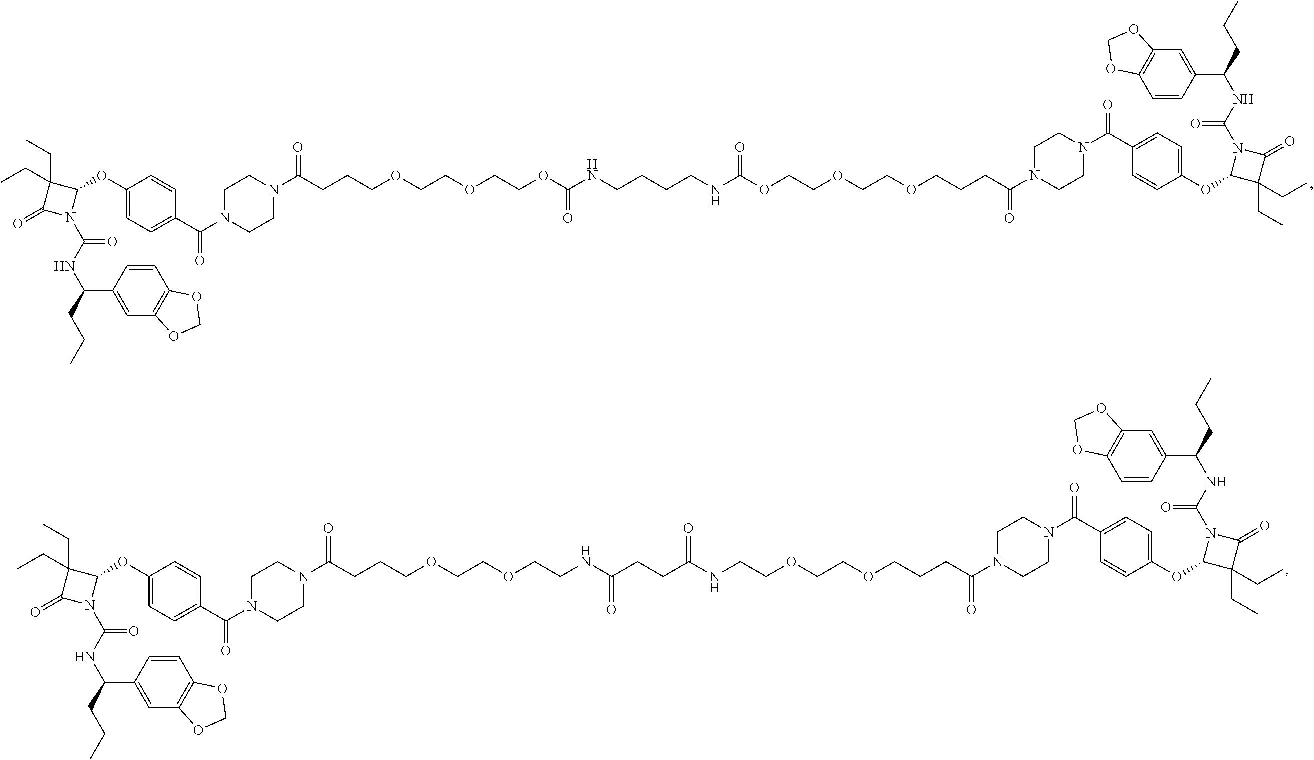

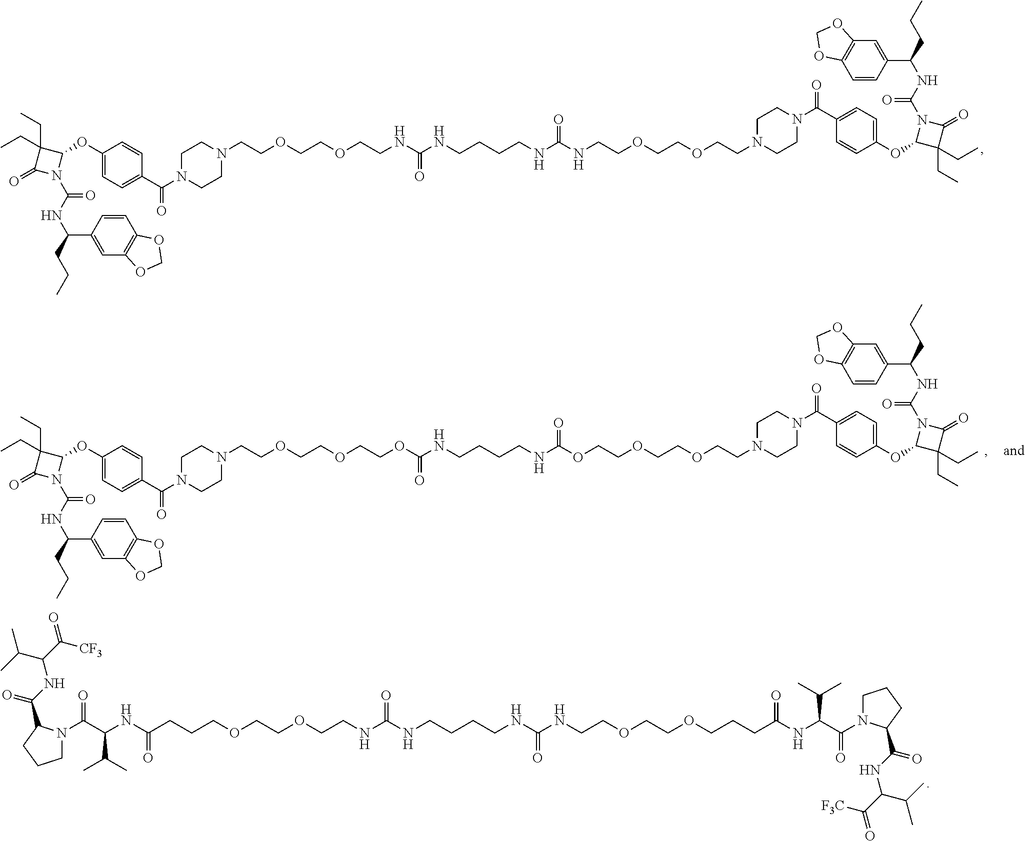

### Preparation of Compounds

Described herein are compounds of Formula (I), (Ia), (II), (III), (IV), (V), (VI), (VII), (VIII), or (IX) that treat a lung disease, and processes for their preparation. Also described herein are compounds of Formula (I), (Ia), (II), (III), (IV), (V), (VI), (VII), (VIII), or (IX) that treat an intestinal disease, and processes for their preparation. Also described herein are pharmaceutically acceptable salts, pharmaceutically acceptable solvates, pharmaceutically active metabolites, and pharmaceutically acceptable prodrugs of such compounds. Pharmaceutical compositions comprising at least one such compound or a pharmaceutically acceptable salt, pharmaceutically acceptable solvate, pharmaceutically active metabolite or pharmaceutically acceptable prodrug of such compound, and a pharmaceutically acceptable excipient are also provided.

Also described herein are compounds of Formula (I), (Ia), (II), (III), (IV), (V), (VI), (VII), (VIII), or (IX) that treat COPD or cystic fibrosis, and processes for their preparation. In some embodiments described herein are compounds of Formula (I), (Ia), (II), (III), (IV), (V), (VI), (VII), (VIII), or (IX) that treat COPD, and processes for their preparation. In some embodiments described herein are compounds of Formula (I), (Ia), (II), (III), (IV), (V), (VI), (VII), (VIII), or (IX) that treat cystic fibrosis, and processes for their preparation. Also described herein are compounds of Formula (I), (Ia), (II), (III), (IV), (V), (VI), (VII), (VIII), or (IX) that treat IBD, and processes for their preparation. Also described herein are pharmaceutically acceptable salts, pharmaceutically acceptable solvates, pharmaceutically active metabolites, and pharmaceutically acceptable prodrugs of such compounds. Pharmaceutical compositions comprising at least one such compound or a pharmaceutically acceptable salt, pharmaceutically acceptable solvate, pharmaceutically active metabolite or pharmaceutically acceptable prodrug of such compound, and a pharmaceutically acceptable excipient are also provided.

Compounds of Formula (I), (Ia), (II), (III), (IV), (V), (VI), (VII), (VIII), or (IX) may be synthesized using standard synthetic reactions known to those of skill in the art or using methods known in the art. The reactions can be employed in a linear sequence to provide the compounds or they may be used to synthesize fragments which are subsequently joined by the methods known in the art.

The starting material used for the synthesis of the compounds described herein may be synthesized or can be obtained from commercial sources, such as, but not limited to, Aldrich Chemical Co. (Milwaukee, Wis.), Bachem (Torrance, Calif.), or Sigma Chemical Co. (St. Louis, Mo.). The compounds described herein, and other related compounds having different substituents can be synthesized using techniques and materials known to those of skill in the art, such as described, for example, in March, ADVANCED ORGANIC CHEMISTRY 4^{th} Ed., (Wiley 1992); Carey and Sundberg, ADVANCED ORGANIC CHEMISTRY 4^{th} Ed., Vols. A and B (Plenum 2000, 2001); Green and Wuts, PROTECTIVE GROUPS IN ORGANIC SYNTHESIS 3^{rd} Ed., (Wiley 1999); Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); and Larock's Comprehensive Organic Transformations (VCH Publishers Inc., 1989); (all of which are incorporated by reference in their entirety). General methods for the preparation of compound as disclosed herein may be derived from known reactions in the field, and the reactions may be modified by the use of appropriate reagents and conditions, as would be recognized by the skilled person, for the introduction of the various moieties found in the formulae as provided herein.

The products of the reactions may be isolated and purified, if desired, using conventional techniques, including, but not limited to, filtration, distillation, crystallization, chromatography and the like. Such materials may be characterized using conventional means, including physical constants and spectral data.

Compounds described herein may be prepared as a single isomer or a mixture of isomers.

The starting materials and intermediates for the compounds of this invention may be prepared by the application or adaptation of the methods described below, their obvious chemical equivalents, or, for example, as described in literature such as The Science of Synthesis, Volumes 1-8. Editors E. M. Carreira et al. Thieme publishers (2001-2008). Details of reagent and reaction options are also available by structure and reaction searches using commercial computer search engines such as SciFinder or Reaxys.

A representative compound of Formula (I), can be prepared as depicted in Schemes 1-3.

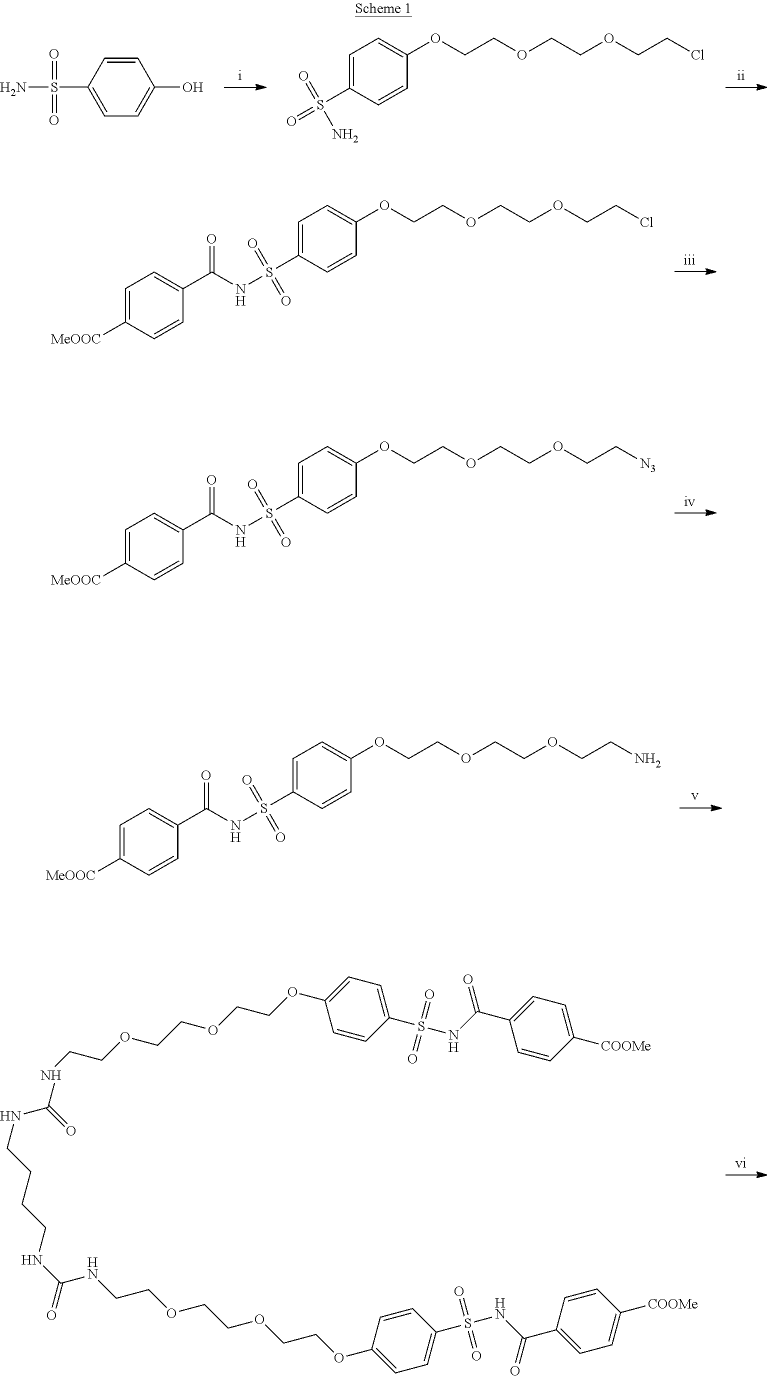

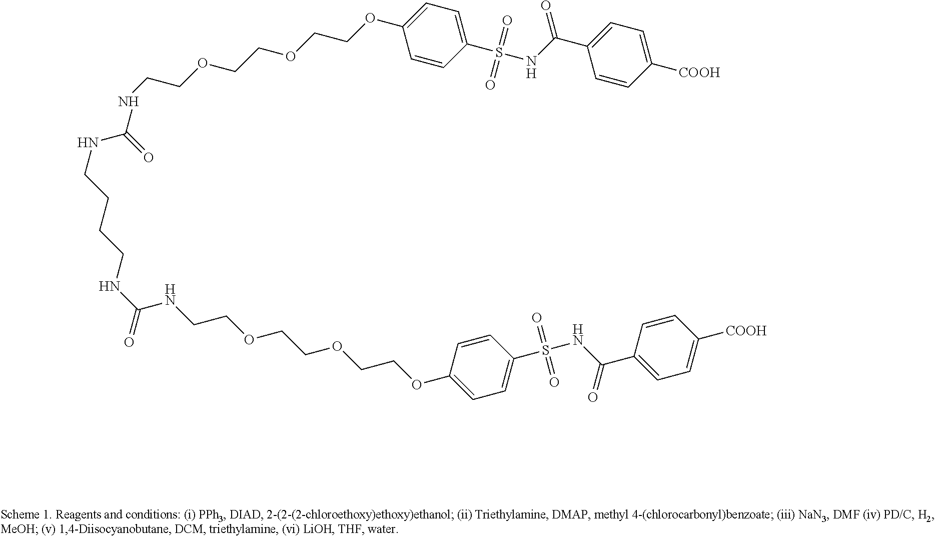 [SMILES: O=C(NCCCCNC(=O)NCCOCCOCCOc1ccc(S(=O)(=O)NC(=O)c2ccc(C(=O)O)cc2)cc1)NCCOCCOCCOc1ccc(S(=O)(=O)NC(=O)c2ccc(C(=O)O)cc2)cc1]

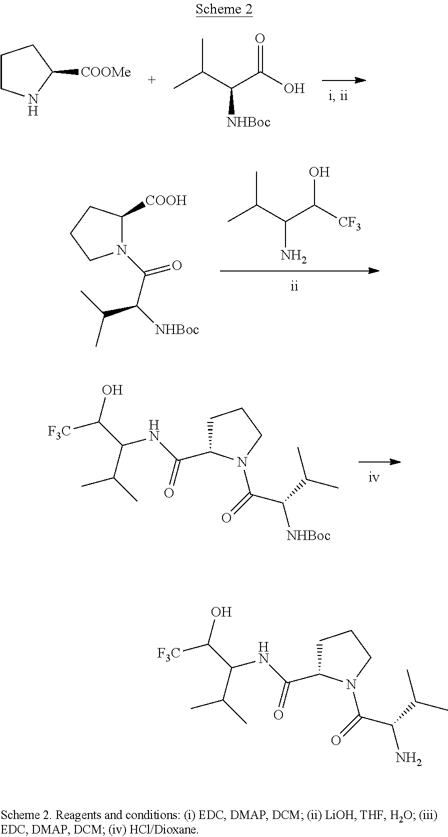

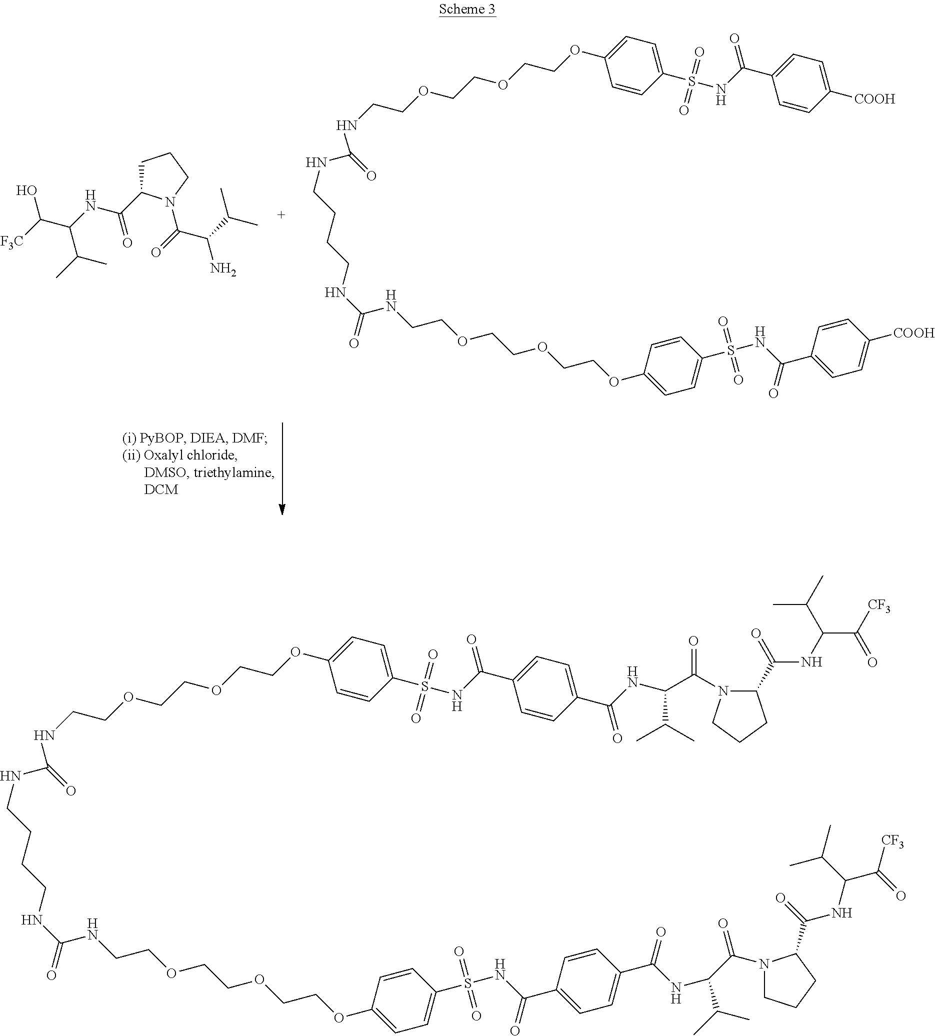 [SMILES: CC(C)C(NC(=O)[C@@H]1CCCN1C(=O)[C@@H](N)C(C)C)C(O)C(F)(F)F ; O=C(NCCCCNC(=O)NCCOCCOCCOc1ccc(S(=O)(=O)NC(=O)c2ccc(C(=O)O)cc2)cc1)NCCOCCOCCOc1ccc(S(=O)(=O)NC(=O)c2ccc(C(=O)O)cc2)cc1 ; CC(C)C(NC(=O)[C@@H]1CCCN1C(=O)[C@@H](NC(=O)c1ccc(C(=O)NS(=O)(=O)c2ccc(OCCOCCOCCNC(=O)NCCCCNC(=O)NCCOCCOCCOc3ccc(S(=O)(=O)NC(=O)c4ccc(C(=O)N[C@H](C(=O)N5CCC[C@H]5C(=O)NC(C(=O)C(F)(F)F)C(C)C)C(C)C)cc4)cc3)cc2)cc1)C(C)C)C(=O)C(F)(F)F]

A second representative compound of Formula (I) can be prepared as depicted in Scheme 4.

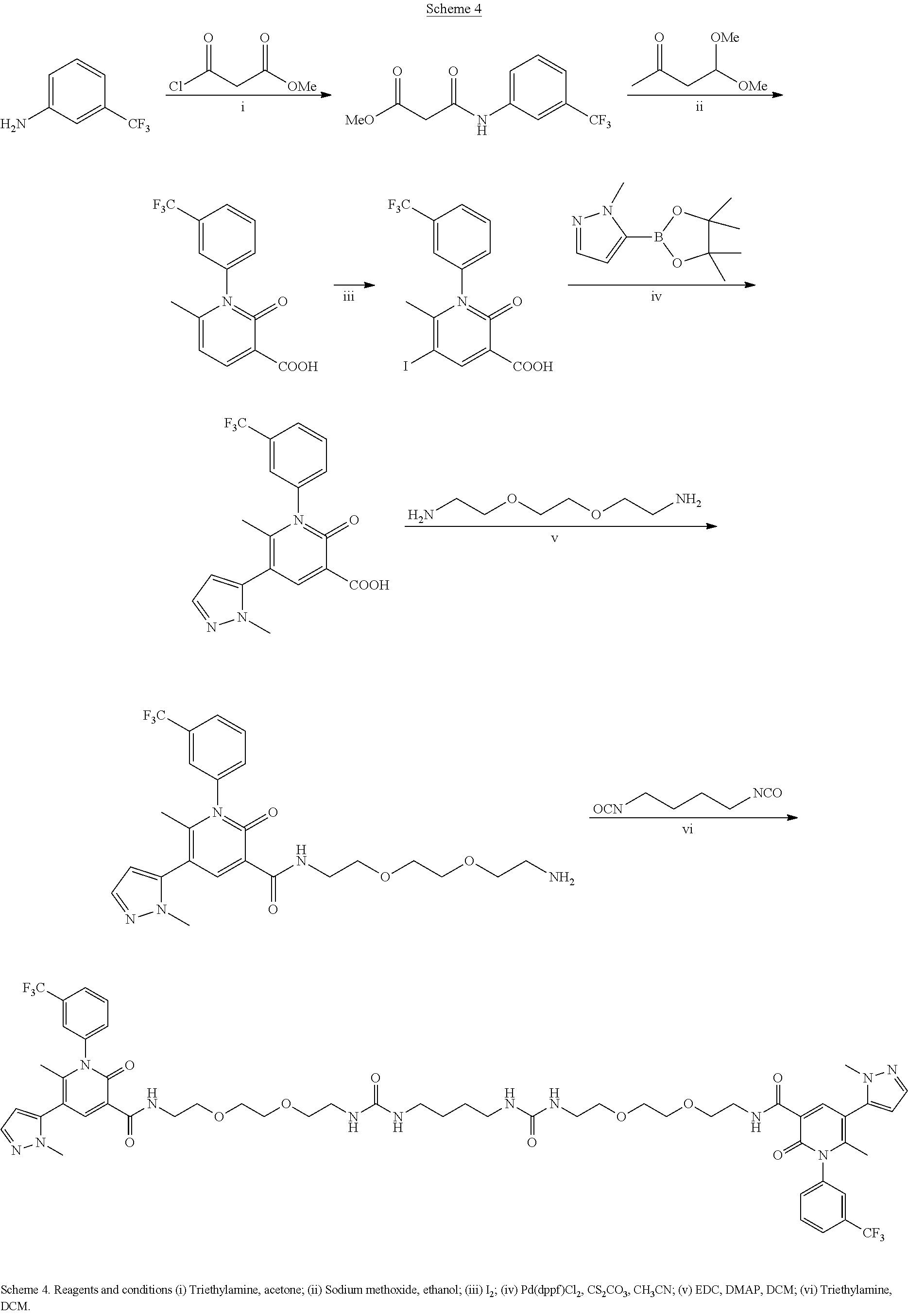

### Further Forms of Compounds Disclosed Herein

### Isomers

Furthermore, in some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, compounds exist as tautomers. The compounds described herein include all possible tautomers within the formulas described herein. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred (e.g., crystalline diastereomeric salts). In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Labeled Compounds

In some embodiments, the compounds described herein exist in their isotopically-labeled forms. In certain embodiments, the compounds described herein exist as partially or fully deuterated forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, sulfur, fluorine and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the metabolites, pharmaceutically acceptable salts, esters, prodrugs, solvate, hydrates or derivatives thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i. e., ³H and carbon-14, i. e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements. In some embodiments, the isotopically labeled compounds, pharmaceutically acceptable salt, ester, prodrug, solvate, hydrate or derivative thereof is prepared by any suitable method.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds of the invention, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate undeconate and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds of the invention and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. By way of example only, hydrates of the compounds described herein can be conveniently prepared by recrystallization from an aqueous/organic solvent mixture, using organic solvents including, but not limited to, dioxane, tetrahydrofuran or methanol. In addition, the compounds provided herein can exist in unsolvated as well as solvated forms. In general, the solvated forms are considered equivalent to the unsolvated forms for the purposes of the compounds and methods provided herein.

### Polymorphs

In some embodiments, the compounds described herein exist as polymorphs. The invention provides for methods of treating diseases by administering such polymorphs. The invention further provides for methods of treating diseases by administering such polymorphs as pharmaceutical compositions.

Thus, the compounds described herein include all their crystalline forms, known as polymorphs. Polymorphs include the different crystal packing arrangements of the same elemental composition of a compound. In certain instances, polymorphs have different X-ray diffraction patterns, infrared spectra, melting points, density, hardness, crystal shape, optical and electrical properties, stability, and solubility. In certain instances, various factors such as the recrystallization solvent, rate of crystallization, and storage temperature cause a single crystal form to dominate.

### Prodrugs

In some embodiments, the compounds described herein exist in prodrug form. The invention provides for methods of treating diseases by administering such prodrugs. The invention further provides for methods of treating diseases by administering such prodrugs as pharmaceutical compositions.

Prodrugs are generally drug precursors that, following administration to an individual and subsequent absorption, are converted to an active, or a more active species via some process, such as conversion by a metabolic pathway. Some prodrugs have a chemical group present on the prodrug that renders it less active and/or confers solubility or some other property to the drug. Once the chemical group has been cleaved and/or modified from the prodrug the active drug is generated. Prodrugs are often useful because, in some situations, they are easier to administer than the parent drug. They are, for instance, bioavailable by oral administration whereas the parent is not. In certain instances, the prodrug also has improved solubility in pharmaceutical compositions over the parent drug. An example, without limitation, of a prodrug would be a compound as described herein which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyamino acid) bonded to an acid group where the peptide is metabolized to reveal the active moiety. (See for example Bundgaard, “Design and Application of Prodrugs” in A Textbook of Drug Design and Development, Krosgaard-Larsen and Bundgaard, Ed., 1991, Chapter 5, 113-191, which is incorporated herein by reference).

In some embodiments, prodrugs are designed as reversible drug derivatives, for use as modifiers to enhance drug transport to site-specific tissues. The design of prodrugs to date has been to increase the effective water solubility of the therapeutic compound for targeting to regions where water is the principal solvent.

Additionally, prodrug derivatives of compounds described herein can be prepared by methods described herein are otherwise known in the art (for further details see Saulnier et al., Bioorganic and Medicinal Chemistry Letters, 1994, 4, 1985). By way of example only, appropriate prodrugs can be prepared by reacting a non-derivatized compound with a suitable carbamylating agent, such as, but not limited to, 1,1-acyloxyalkylcarbanochloridate, para-nitrophenyl carbonate, or the like. Prodrug forms of the herein described compounds, wherein the prodrug is metabolized in vivo to produce a derivative as set forth herein are included within the scope of the claims. Indeed, some of the herein-described compounds are prodrugs for another derivative or active compound.

In some embodiments, prodrugs include compounds wherein an amino acid residue, or a polypeptide chain of two or more (e. g., two, three or four) amino acid residues is covalently joined through an amide or ester bond to a free amino, hydroxy or carboxylic acid group of compounds of the present invention. The amino acid residues include but are not limited to the 20 naturally occurring amino acids and also includes 4-hydroxyproline, hydroxylysine, demosine, isodemosine, 3-methylhistidine, norvaline, beta-alanine, gamma-aminobutyric acid, cirtulline, homocysteine, homoserine, ornithine and methionine sulfone. In other embodiments, prodrugs include compounds wherein a nucleic acid residue, or an oligonucleotide of two or more (e. g., two, three or four) nucleic acid residues is covalently joined to a compound of the present invention.

Pharmaceutically acceptable prodrugs of the compounds described herein also include, but are not limited to, esters, carbonates, thiocarbonates, N-acyl derivatives, N-acyloxyalkyl derivatives, quaternary derivatives of tertiary amines, N-Mannich bases, Schiff bases, amino acid conjugates, phosphate esters, metal salts and sulfonate esters. Compounds having free amino, amido, hydroxy or carboxylic groups can be converted into prodrugs. For instance, free carboxyl groups can be derivatized as amides or alkyl esters. In certain instances, all of these prodrug moieties incorporate groups including but not limited to ether, amine and carboxylic acid functionalities.

Hydroxy prodrugs include esters, such as though not limited to, acyloxyalkyl (e.g. acyloxymethyl, acyloxyethyl) esters, alkoxycarbonyloxyalkyl esters, alkyl esters, aryl esters, phosphate esters, sulfonate esters, sulfate esters and disulfide containing esters; ethers, amides, carbamates, hemisuccinates, dimethylaminoacetates and phosphoryloxymethyloxycarbonyls, as outlined in Advanced Drug Delivery Reviews 1996, 19, 115.

Amine derived prodrugs include, but are not limited to the following groups and combinations of groups:

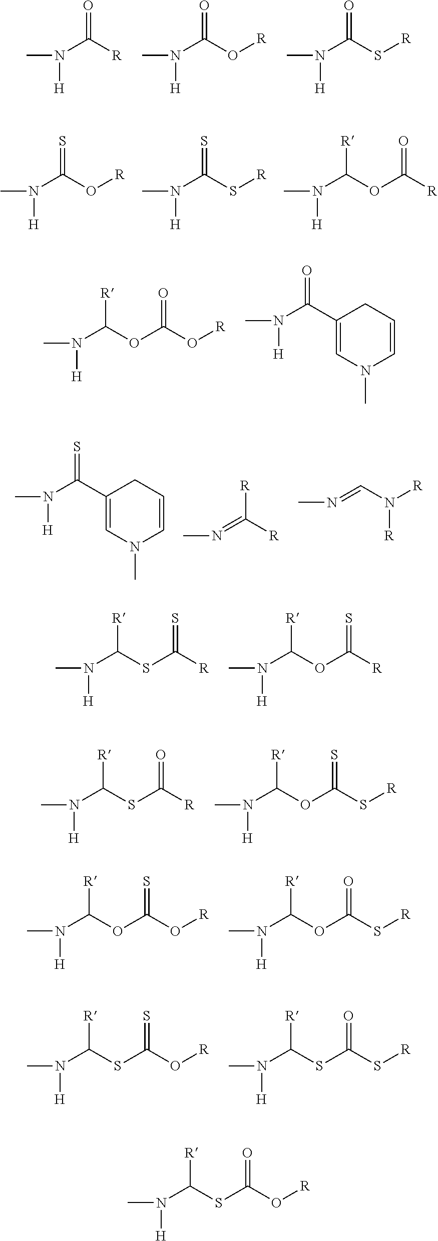

as well as sulfonamides and phosphonamides.

In certain instances, sites on any aromatic ring portions are susceptible to various metabolic reactions, therefore incorporation of appropriate substituents on the aromatic ring structures, can reduce, minimize or eliminate this metabolic pathway.

### Metabolites

In some embodiments, compounds of Formula (I), (Ia), (II), (III), (IV), (V), (VI), (VII), (VIII), or (IX) are susceptible to various metabolic reactions. Therefore, in some embodiments, incorporation of appropriate substituents into the structure will reduce, minimize, or eliminate a metabolic pathway. In specific embodiments, the appropriate substituent to decrease or eliminate the susceptibility of an aromatic ring to metabolic reactions is, by way of example only, a halogen, or an alkyl group.

In additional or further embodiments, the compounds of Formula (I), (Ia), (II), (III), (IV), (V), (VI), (VII), (VIII), or (IX) described herein are metabolized upon administration to an organism in need to produce a metabolite that is then used to produce a desired effect, including a desired therapeutic effect.

### Pharmaceutical Compositions/Formulations

In another aspect, provided herein are pharmaceutical composition comprising a compound of Formula (I), (Ia), (II), (III), (IV), (V), (VI), (VII), (VIII), or (IX) as described herein, or a pharmaceutically acceptable salt, polymorph, solvate, prodrug, metabolite, deuteride, N-oxide, stereoisomer, or isomer thereof, and a pharmaceutically acceptable excipient.

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999), herein incorporated by reference for such disclosure.

Provided herein are pharmaceutical compositions that include a compound of Formula (I), (Ia), (II), (III), (IV), (V), (VI), (VII), (VIII), or (IX) and at least one pharmaceutically acceptable inactive ingredient. In some embodiments, the compounds described herein are administered as pharmaceutical compositions in which a compound of Formula (I), (Ia), (II), (III), (IV), (V), (VI), (VII), (VIII), or (IX) is mixed with other active ingredients, as in combination therapy. In other embodiments, the pharmaceutical compositions include other medicinal or pharmaceutical agents, carriers, adjuvants, preserving, stabilizing, wetting or emulsifying agents, solution promoters, salts for regulating the osmotic pressure, and/or buffers. In yet other embodiments, the pharmaceutical compositions include other therapeutically valuable substances.

A pharmaceutical composition, as used herein, refers to a mixture of a compound of Formula (I), (Ia), (II), (III), (IV), (V), (VI), (VII), (VIII), or (IX) with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. The pharmaceutical composition facilitates administration of the compound to an organism. In practicing the methods of treatment or use provided herein, therapeutically effective amounts of compounds described herein are administered in a pharmaceutical composition to a mammal having a disease, disorder, or condition to be treated. In some embodiments, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures.

The pharmaceutical formulations described herein are administered to a subject by appropriate administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, liquids, gels, syrups, elixirs, slurries, suspensions, self-emulsifying dispersions, solid solutions, liposomal dispersions, aerosols, solid oral dosage forms, powders, immediate release formulations, controlled release formulations, fast melt formulations, tablets, capsules, pills, powders, dragees, effervescent formulations, lyophilized formulations, delayed release formulations, extended release formulations, pulsatile release formulations, multiparticulate formulations, and mixed immediate and controlled release formulations.

In some embodiments described herein is a pharmaceutical composition comprising a compound of Formula (I), (Ia), (II), (III), (IV), (V), (VI), (VII), (VIII), or (IX), and a pharmaceutically acceptable excipient, where the compound is in a formulation suitable for delivery by inhalation. The inhalational route of administration provides the advantage that the inhaled drug can directly target the lung. An inhalational pharmaceutical delivery system is one that is suitable for respiratory therapy by delivery of an active agent to mucosal linings of the bronchi. This invention can utilize a system that depends on the power of a compressed gas to expel an active agent from a container. An aerosol or pressurized package can be employed for this purpose.

As used herein, the term “aerosol” is used in its conventional sense as referring to very fine liquid or solid particles carries by a propellant gas under pressure to a site of therapeutic application. When a pharmaceutical aerosol is employed in the context of the present disclosure, the aerosol contains an active agent, which can be dissolved, suspended, or emulsified in a mixture of a fluid carrier and a propellant. The aerosol can be in the form of a solution, suspension, emulsion, powder, or semi-solid preparation. Aerosols employed in the present invention are intended for administration as fine, solid particles or as liquid mists via the respiratory tract of a patient. Various types of propellants known to one of skill in the art can be utilized. Suitable propellants include, but are not limited to, hydrocarbons or other suitable gas. In the case of the pressurized aerosol, the dosage unit may be determined by providing a value to deliver a metered amount.

In some embodiments a compound of Formula (I), (Ia), (II), (III), (IV), (V), (VI), (VII), (VIII), or (IX) is formulated for delivery with a nebulizer, which is an instrument that generates very fine liquid particles of substantially uniform size in a gas. For example, a liquid containing an active agent is dispersed as droplets. The small droplets can be carried by a current of air through an outlet tube of the nebulizer. The resulting mist penetrates into the respiratory tract of the patient.

In some embodiments, a powder composition containing a compound of Formula (I), (Ia), (II), (III), (IV), (V), (VI), (VII), (VIII), or (IX), with or without a lubricant, carrier, or propellant, can be administered to a mammal in need of therapy. This embodiment of the invention can be carried out with a conventional device for administering a powder pharmaceutical composition by inhalation. For example, a powder mixture of the compound and a suitable powder base such as lactose or starch may be presented in unit dosage form in for example capsular or cartridges, e.g. gelatin, or blister packs, from which the powder may be administered with the aid of an inhaler.

Pharmaceutical compositions including a compound of Formula (I), (Ia), (II), (III), (IV), (V), (VI), (VII), (VIII), or (IX) are manufactured in a conventional manner, such as, by way of example only, by means of conventional mixing, dissolving, granulating, dragee-making, levigating, emulsifying, encapsulating, entrapping or compression processes.

The pharmaceutical compositions will include at least one compound of Formula (I), (Ia), (II), (III), (IV), (V), (VI), (VII), (VIII), or (IX) as an active ingredient in free-acid or free-base form, or in a pharmaceutically acceptable salt form. In addition, the methods and pharmaceutical compositions described herein include the use of N-oxides (if appropriate), crystalline forms, amorphous phases, as well as active metabolites of these compounds having the same type of activity. In some embodiments, compounds described herein exist in unsolvated form or in solvated forms with pharmaceutically acceptable solvents such as water, ethanol, and the like. The solvated forms of the compounds presented herein are also considered to be disclosed herein.

Pharmaceutical preparations for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross-linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active compound doses.

Pharmaceutical preparations that are administered orally include push-fit capsules made of gelatin, as well as soft, sealed capsules made of gelatin and a plasticizer, such as glycerol or sorbitol. The push-fit capsules contain the active ingredients in admixture with filler such as lactose, binders such as starches, and/or lubricants such as talc or magnesium stearate and, optionally, stabilizers. In soft capsules, the active compounds are dissolved or suspended in suitable liquids, such as fatty oils, liquid paraffin, or liquid polyethylene glycols. In some embodiments, stabilizers are added.

In certain embodiments, delivery systems for pharmaceutical compounds may be employed, such as, for example, liposomes and emulsions. In certain embodiments, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

Any conventional carrier or excipient may be used in the pharmaceutical compositions of the embodiments. The choice of a particular carrier or excipient, or combinations of carriers or excipients, will depend on the mode of administration being used to treat a particular patient or type of medical condition or disease state.

### Combination Treatment

The compounds according to Formula (I), (Ia), (II), (III), (IV), (V), (VI), (VII), (VIII), or (IX) may be used in combination with one or more additional antibiotic agents, recombinant human DNase, hypertonic saline, anti-inflammatory agents and bronchodilators.

The antibiotic agent may be selected from an aminoglycoside, ansamycin, carbacephem, carbapenem, cephalosporin, glycopeptide, lincosamide, lipopeptide, macrolide, monobactam, nitrofurans, penicillin, polypeptide, quinolone, sulfonamide, or tetracycline antibiotic. Examples of antibiotic agents include, but are not limited to, Aminoglycoside derivatives like amikacin, gentamicin, kanamycin, neomycin, netilmicin, tobramicin, paromomycin; Ansamycin derivatives like geldanamycin, herbimycin; Carbacephem derivatives like loracarbef, Carbapenem derivatives like ertapenem, doripenem, imipenem, meropenem; Cephalosporin derivatives like cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftobiprole; Glycopeptide derivatives like teicoplanin, vancomycin, telavancin; Lincosamides like clindamycin, lincomycin; Lipopeptide derivatives like daptomycin; Macrolide derivatives like azithromycin, clarithromycin, dirithromycin, erythromycin, roxithromycin, troleandomycin; telithreomycin, spectinomycin; Monobactam derivatives like aztreonam; Nitrofuran derivatives like furazolidone, nitrofurantoin; Penicillin derivatives like amoxicillin, ampicillin, azlocillin, carbinicillin, cloxacillin, dicloxacillin, flucloxacillin, mezlocillin, methicillin, nafcillin, oxacillin, penicillin G, penicillin V, piperacillin, temocillin, ticarcillin; Penicillin combinations like amoxicillin/clavulanate, ampicillin/sulbactam, piperacillin/tazobactam, ticarcillin/clavulanate; Polypeptide derivatives like bacitracin, colistin, polymyxin B; Quinolone derivatives like ciprofloxacin, enoxacin, gatifloxacin, levofloxacin, lomefloxacin, moxifloxacin, nalidixic acid, norfloxacin, ofloxacin, trovafloxacin, grepafloxacin, sparfloxacin, temafloxacin; Sulfonamide derivatives like mafenide, sulfonamidochrysoidine, sulfacetamide, sulfadiazine, silver sulfadiazine, sulfamethoxazole, sulfanilimide, sulfasalazine, sulfisoxazole, trimethoprim, trimethoprim/sulfamethoxazole; Tetracyclin derivatives like demeclocycline, doxycycline, minocycline, oxytetracycline, tetracycline; Derivatives against mycobacteria like clofazimine, dapsone, capreomycin, cycloserine, ethambutol, ethioamide, isoniazid, pyrazinamide, rifampin, refampicin, rifabutin, rifapentine, streptomycin; or other antibiotic agents like arsphenamine, chloramphenicol, fosfomycin, fusidic acid, linezolid, metronidazole, mupirocin, platensimycin, quinupristin/dalfopristin, rifaximin, thiampheniol, tigecycline, tinidazole.

Examples of bronchodilator agents include, but are not limited to, β₂-adrenergic receptor agonists such as albuterol, levalbuterol, pirbuterol, epinephrine, ephedrine, terbutaline, salmeterol, clenbuterol, formoterol, bambuterol, and indacaterol.

Anti-inflammatory agents include, but are not limited to, NSAIDS and glucocorticoids. Non-limiting examples of NSAIDS include aspirin, diflunisal, salsalate, ibuprofen, dexibuprofen, naproxen, fenoprofen, ketoprofen, dexketoprofen, flurbiprofen, oxaprozin, loxoprofen, indomethacin, tolmetin, sulindac, etodolac, ketorolac, nabumetone, diclofenac, piroxicam, meloxicam, tenoxicam, droxicam, lornoxicam, isoxicam, mefenamic acid, meclofenamic acid, flufenamic acid, tolfenamic acid, celecoxib, parecoxib, etoricoxib, lumiracoxib, and firocoxib. Glucocorticoids include, but are not limited to, hydrocortisone, cortisone, prednisone, prednisolone, methylprednisolone, dexamethasone, betamethasone, triamcinolone, beclometasone, or fludrocortisone.

### Administration of Pharmaceutical Composition

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In some embodiments, compounds of Formula (I), (Ia), (II), (III), (IV), (V), (VI), (VII), (VIII), or (IX) and compositions thereof are administered in any suitable manner. The manner of administration can be chosen based on, for example, whether local or systemic treatment is desired, and on the area to be treated. For example, the compositions can be administered orally, parenterally (e.g., intravenous, subcutaneous, intraperitoneal, or intramuscular injection), by inhalation, extracorporeally, topically (including transdermally, ophthalmically, vaginally, rectally, intranasally) or the like.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained.

Further described herein are pharmaceutical compositions comprising a compound of Formula (I), (Ia), (II), (III), (IV), (V), (VI), (VII), (VIII), or (IX) utilized for administration wherein absorption is limited such that the compound is rendered substantially impermeable or substantially systemically non-bioavailable by means of increasing the molecular weight of the compound. In some embodiments, the compound of Formula (I), (Ia), (II), (III), (IV), (V), (VI), (VII), (VIII), or (IX) may be rendered substantially impermeable or substantially systemically non-bioavailable by means of increasing the polar surface area.

For therapeutic use in intestinal diseases, local delivery to the intestine can be carried out. Compositions highly selective or localized in the intestine act substantially in the intestine without exposure to other tissues or organs. These compositions are substantially systemically non-bioavailable or substantially impermeable to the epithelial layer covering the internal surface of the gastrointestinal tract. In this way, any systemic effects can be minimized.

For therapeutic use in lung diseases, local delivery to the lung can be carried out. Delivery by inhalation or insufflating aerosols provides high level concentrations of drug compared to the concentration absorbed systemically. Compositions highly selective or localized in the lung act substantially in the lung without exposure to other tissues or organs. In this way, any systemic effects can be minimized. In addition, administration by inhalation can provide for smaller doses delivered locally to the specific cells in the lung which are most in need of treatment. By delivering smaller doses, any adverse side effects are eliminated or substantially reduced. By delivering directly to the cells which are most in need of treatment, the effect of the treatment will be realized more quickly.

In some embodiments described herein, are pharmaceutical compositions comprising a compound of Formula (I), (Ia), (II), (III), (IV), (V), (VI), (VII), (VIII), or (IX) and a pharmaceutically acceptable excipient, where the compound is in a formulation suitable for administration by inhalation, e.g., inhalation into the lungs.

The compound of Formula (I), (Ia), (II), (III), (IV), (V), (VI), (VII), (VIII), or (IX) may be administered to the subject by means of a pharmaceutical delivery system for the inhalation route. The compounds may be formulated in a form suitable for administration by inhalation. The pharmaceutical delivery system is one that is suitable for respiratory therapy by administration via inhalation of a compound of any one of Formula (I), (Ia), (II), (III), (IV), (V), (VI), (VII), (VIII), or (IX) thereof to the lung.

In certain embodiments, the pharmaceutical compositions are suitable for inhaled administration. Suitable pharmaceutical compositions for inhaled administration will typically be in the form of an aerosol or a powder. Such compositions are generally administered using well-known delivery devices, such as a nebulizer inhaler, a metered-dose inhaler (MDI), a dry powder inhaler (DPI) or a similar delivery device. When a pharmaceutical aerosol is employed in this invention, the aerosol contains the therapeutically active compound, which can be dissolved, suspended, or emulsified in a mixture of a fluid carrier and a propellant. The aerosol can be in the form of a solution, suspension, emulsion, powder, or semi-solid preparation. Aerosols employed in the present embodiments are intended for administration as fine, solid particles or as liquid mists via the respiratory tract of a patient. Various types of propellants known to one of skill in the art can be utilized. Examples of suitable propellants include, but are not limited to, hydrocarbons or other suitable gas. In the case of the pressurized aerosol, the dosage unit may be determined by providing a value to deliver a metered amount.

In certain embodiments, the pharmaceutical composition comprising a compound of Formula (I), (Ia), (II), (III), (IV), (V), (VI), (VII), (VIII), or (IX) is administered by inhalation using a nebulizer inhaler. Such nebulizer devices typically produce a stream of high velocity air that causes the pharmaceutical composition comprising the active agent to spray as a mist that is carried into the patient's respiratory tract. Accordingly, when formulated for use in a nebulizer inhaler, the active agent is typically dissolved in a suitable carrier to form a solution. Alternatively, the active agent can be micronized and combined with a suitable carrier to form a suspension of micronized particles of respirable size.

In certain embodiments, the pharmaceutical composition comprising a compound of Formula (I), (Ia), (II), (III), (IV), (V), (VI), (VII), (VIII), or (IX) is administered by inhalation using a dry powder inhaler. Such dry powder inhalers typically administer the active agent as a free-flowing powder that is dispersed in a patient's air-stream during inspiration. In order to achieve a free-flowing powder, the active agent is typically formulated with a suitable excipient such as lactose or starch.

In certain embodiments, the pharmaceutical composition comprising a compound of Formula (I), (Ia), (II), (III), (IV), (V), (VI), (VII), (VIII), or (IX) is administered by inhalation using a metered-dose inhaler. Such metered-dose inhalers typically discharge a measured amount of the active agent or a pharmaceutically acceptable salt thereof using compressed propellant gas. Accordingly, pharmaceutical compositions administered using a metered-dose inhaler can comprise a solution or suspension of the active agent in a liquefied propellant.

### Methods

Provided herein is a method to treat a lung disease in a mammal, the method comprising administering to the mammal a compound of Formula (I), (Ia), (II), (III), (IV), (V), (VI), (VII), (VIII), or (IX), or a pharmaceutically acceptable salt, solvate, polymorph, prodrug, metabolite, deuteride, N-oxide, stereoisomer, or isomer thereof, as described above and below.

Also provided herein is a method to treat COPD or cystic fibrosis in a mammal, the method comprising administering to the mammal a compound of Formula (I), (Ia), (II), (III), (IV), (V), (VI), (VII), (VIII), or (IX), or a pharmaceutically acceptable salt, solvate, polymorph, prodrug, metabolite, deuteride, N-oxide, stereoisomer, or isomer thereof, as described above and below. In some embodiments is a method to treat COPD in a mammal, the method comprising administering to the mammal a compound of Formula (I), (Ia), (II), (III), (IV), (V), (VI), (VII), (VIII), or (IX), or a pharmaceutically acceptable salt, solvate, polymorph, prodrug, metabolite, deuteride, N-oxide, stereoisomer, or isomer thereof, as described above and below. In some embodiments is a method to treat cystic fibrosis in a mammal, the method comprising administering to the mammal a compound of Formula (I), (Ia), (II), (III), (IV), (V), (VI), (VII), (VIII), or (IX), or a pharmaceutically acceptable salt, solvate, polymorph, prodrug, metabolite, deuteride, N-oxide, stereoisomer, or isomer thereof, as described above and below.

Also provided herein is a method to treat an intestinal disease in a mammal, the method comprising administering to the mammal a compound of Formula (I), (Ia), (II), (III), (IV), (V), (VI), (VII), (VIII), or (IX), or a pharmaceutically acceptable salt, solvate, polymorph, prodrug, metabolite, deuteride, N-oxide, stereoisomer, or isomer thereof, as described above and below.

Also provided herein is a method to treat IBD, the method comprising administering to the mammal a compound of Formula (I), (Ia), (II), (III), (IV), (V), (VI), (VII), (VIII), or (IX), or a pharmaceutically acceptable salt, solvate, polymorph, prodrug, metabolite, deuteride, N-oxide, stereoisomer, or isomer thereof, as described above and below.

Further provided herein is the use of a compound of Formula (I), (Ia), (II), (III), (IV), (V), (VI), (VII), (VIII), or (IX), or a pharmaceutically acceptable salt, solvate, polymorph, prodrug, metabolite, deuteride, N-oxide, stereoisomer, or isomer thereof, in the manufacture of a medicament for treating a lung disease.

Also provided herein is the use of a compound of Formula (I), (Ia), (II), (III), (IV), (V), (VI), (VII), (VIII), or (IX), or a pharmaceutically acceptable salt, solvate, polymorph, prodrug, metabolite, deuteride, N-oxide, stereoisomer, or isomer thereof, in the manufacture of a medicament for treating COPD. Also provided herein is the use of a compound of Formula (I), (Ia), (II), (III), (IV), (V), (VI), (VII), (VIII), or (IX), or a pharmaceutically acceptable salt, solvate, polymorph, prodrug, metabolite, deuteride, N-oxide, stereoisomer, or isomer thereof, in the manufacture of a medicament for treating cystic fibrosis.

Also provided herein is the use of a compound of Formula (I), (Ia), (II), (III), (IV), (V), (VI), (VII), (VIII), or (IX), or a pharmaceutically acceptable salt, solvate, polymorph, prodrug, metabolite, deuteride, N-oxide, stereoisomer, or isomer thereof, in the manufacture of a medicament for treating an intestinal disease Also provided herein is the use of a compound of Formula (I), (Ia), (II), (III), (IV), (V), (VI), (VII), (VIII), or (IX), or a pharmaceutically acceptable salt, solvate, polymorph, prodrug, metabolite, deuteride, N-oxide, stereoisomer, or isomer thereof, in the manufacture of a medicament for treating IBD.

### EXAMPLES

### List of Abbreviations

As used above, and throughout the description of the invention, the following abbreviations, unless otherwise indicated, shall be understood to have the following meanings:
ACN acetonitrile
Bn benzyl
BOC or Boc tert-butyl carbamate
BOP benzotriazol-1-yl-oxytris (dimethylamino) phosphonium
t-Bu tert-butyl
Cbz benzyl carbamate
Cy Cyclohexyl
DBU 1,8-Diazabicyclo[5.4.0]undec-7-ene
DCC dicyclohexylcarbodiimide
DCM dichloromethane (CH₂Cl₂)
DIC 1,3-diisopropylcarbodiimide
DEAD diethyl azodicarboxylate
DIAD diisopropyl azodicarboxylate
DIEA diisopropylethylamine
DMAP 4-(N,N-dimethylamino)pyridine
DMP reagent Dess-Martin Periodinane reagent
DMF dimethylformamide
DMA N,N-Dimethylacetamide
DME 1,2-Dimethoxy-ethane
DMSO dimethylsulfoxide
Dppf 1,1′-Bis(diphenylphosphino)ferrocene
EDCI 1-ethyl-3-(3-dimethylaminopropyl) carbodiimide HCl
eq equivalent(s)
Et ethyl
Et₂O diethyl ether
EtOH ethanol
EtOAc ethyl acetate
HOAt 1-hydroxy-7-azabenzotriazole
HOBT 1-hydroxybenztriazole
HOSu N-hydroxysuccinamide
HPLC high performance liquid chromatography
LAH lithium aluminum anhydride
Me methyl
MeI methyliodide
MeOH methanol
MOMCl methoxymethylchloride
MOM methoxymethyl
MS mass spectroscopy
NMM N-methylmorpholine
NMP N-methyl-pyrrolidin-2-one
NMR nuclear magnetic resonance
PyBOP benzotriazole-1-yl-oxytris-pyrrolidino-phosphonium Hexafluorophosphate
SPHOS 2-Dicyclohexylphosphino-2′,6′-dimethoxybiphenyl
TBD 1,5,7-triazabicyclo[4.4.0]-dec-5-ene
RP-HPLC reverse phase-high pressure liquid chromatography
TBS tert-butyldimethylsilyl
TBSCl tert-butyldimethylsilyl chloride
TBTU O-(Benzotriazol-1-yl)-N,N,N′,N′-tetramethyluronium
TEOC 2-Trimethylsilylethyl Carbamate
TFA trifluoroacetic acid
Tf₂O trifluoromethanesulfonic anhydride
TMG 1,1,3,3-Tetramethylguanidine
THF tetrahydrofuran
THP tetrahydropyran
TLC thin layer chromatography
XPHOS 2-Dicyclohexylphosphino-2′,4′,6′-triisopropylbiphenyl

### General Examples for the Preparation of Compounds of the Invention

The starting materials and intermediates for the compounds of this invention may be prepared by the application or adaptation of the methods described below, their obvious chemical equivalents, or, for example, as described in literature such as The Science of Synthesis, Volumes 1-8. Editors E. M. Carreira et al. Thieme publishers (2001-2008). Details of reagent and reaction options are also available by structure and reaction searches using commercial computer search engines such as Scifinder or Reaxys.

### Example 1

### Synthesis of Compound 1

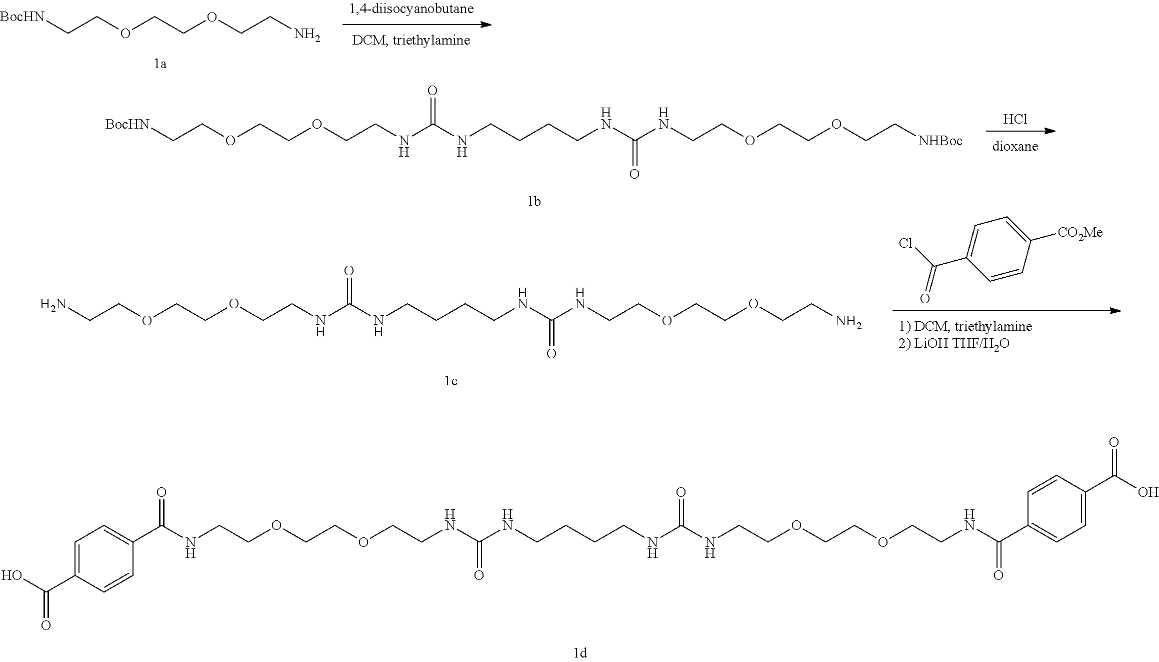 [SMILES: CC(C)(C)OC(=O)NCCOCCOCCN ; CC(C)(C)OC(=O)NCCOCCOCCNC(=O)NCCCCNC(=O)NCCOCCOCCNC(=O)OC(C)(C)C ; Cl ; NCCOCCOCCNC(=O)NCCCCNC(=O)NCCOCCOCCN ; COC(=O)c1ccc(C(=O)Cl)cc1 ; O=C(NCCCCNC(=O)NCCOCCOCCNC(=O)c1ccc(C(=O)O)cc1)NCCOCCOCCNC(=O)c1ccc(C(=O)O)cc1]

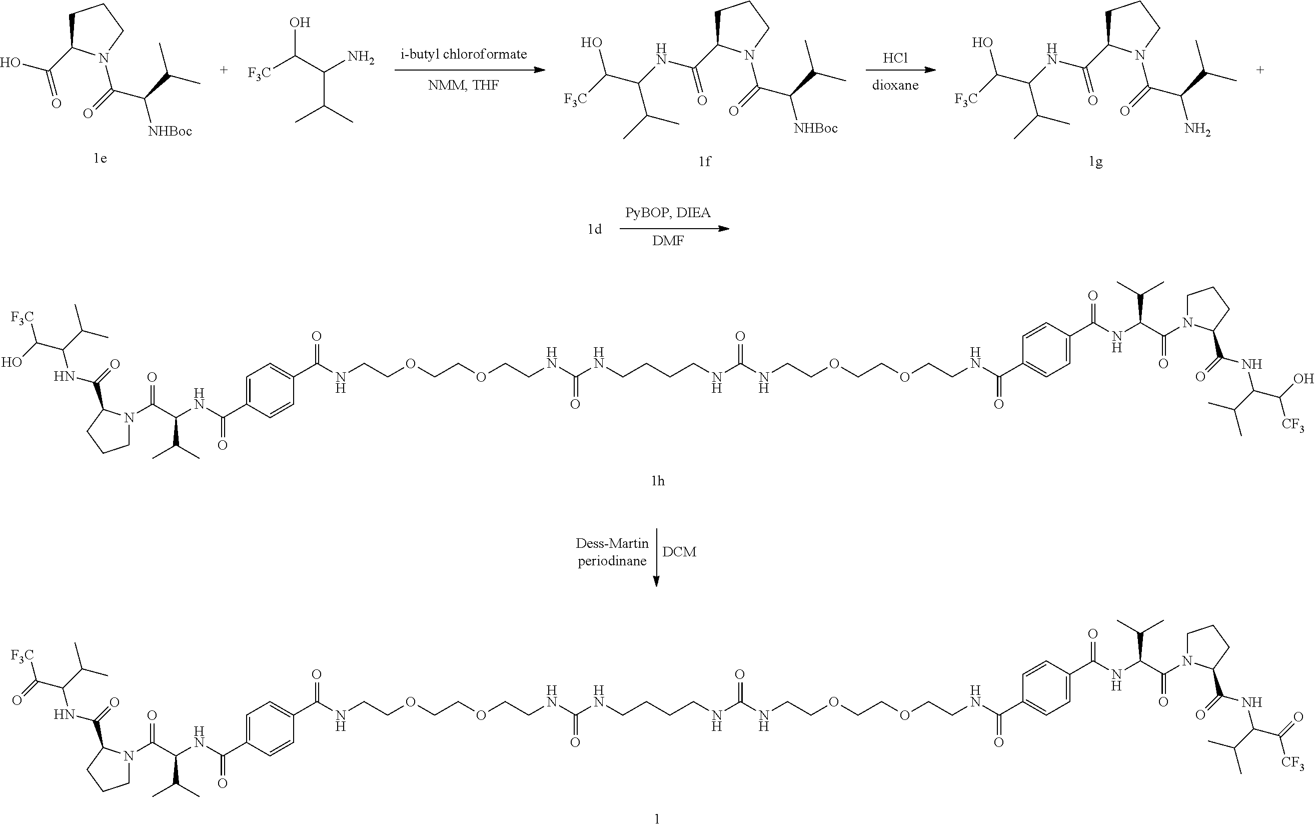

To a solution of tert-butyl 2-(2-(2-aminoethoxy)ethoxy)ethylcarbamate (200 mg, 0.80 mmol) in anhydrous DCM (10 mL) at 0° C. under nitrogen atmosphere was added 1,4-diisocyanobutane (56 mg, 0.40 mmol). To accelerate the reaction a few drops of TEA were added. After addition, the temperature was raised to rt and stirred for 2 h. The product was then concentrated under vacuum and crystallized in ether to give bis-urea linker (1b) (230 mg, 90%). ¹H NMR (400 MHz, CDCl₃) δ 1.46 (s, 9H), 1.57 (s, 2H), 3.26-3.32 (m, 4H), 3.42-3.45 (m, 2H), 3.58-3.64 (m, 8H); MS m/z 637 (M+H).

Compound 1b was dissolved in 4M HCl in 1,4-dioxane solution (5 mL) and stirred at room temperature for 30 minute. The progress of the reaction was monitored by LCMS. After completion of the reaction, solvent was evaporated by vaccum to get crude compound (1c) as a white solid. This crude was crystallized in ether to get the pure compound. ¹H NMR (400 MHz, MeOD) δ 1.51 (s, 2H), 3.09 (t, J=4.8 Hz, 2H), 3.14 (brs, 2H), 3.32 (t, J=4.5 Hz, 2H), 3.53 (t, J=6.2 Hz, 2H), 3.63-3.69 (m, 6H); MS m/z 437 (M+H).

To a solution of compound 1c (140 mg, 0.27 mmol) and triethylamine (138 mg, 1.36 mmol) in dichloromethane at 0° C. was added methyl-4-chlorocarbonylbenzoate (108 mg, 0.54 mmol) portion wise. This reaction mixture was allowed to stir for 2 hours. Progress of the reaction was monitored by LCMS. After completion of the reaction, water (50 mL) added to the reaction mixture, and extracted with dichloromethane (3×50 mL). The combined organic layers were dried under vacuum to afford methyl ester protected linker which was identified by LCMS, in which major peak was showing m/z as 761 M+H. This linker was then dissolved in THF/water mixture (10 mL) and was added lithium hydroxide and stirred at room temperature for 3 hours to afford compound 1d. MS: m/z 733 (M+H).

To a solution of compound 1e (314, 260 mg, mmol) in THF (20 mL) was added NMM (101.5, 100 mg) at 0° C. under nitrogen atmosphere. To this solution isobutyl chloroformate (136.58, 124 mg, mmol) was added dropwise over 5 minutes. This reaction mixture was stirred for 30 minutes at 0° C., then compound 3-amino-1,1,1-trifluoro-4-methylpentan-2-ol (171, 155 mg, mmol) was added and this reaction mixture was stirred for overnight. To the reaction mixture ethyl acetate (100 mL) was added and extracted with water and brine. The organic layers were dried over anhydrous sodium sulfate and concentrated under vacuum to afford compound 1f, MS: m/z 468 (M+H).

Compound 1f was then dissolved in 4M HCl in dioxane (10 mL) and stirred for 1 hour. After completion of the reaction, excess solvent were removed under vacuum and purified by Prep HPLC using a 10-50% linear gradient of acetonitrile in water gradient (0.05% TFA modified mobile phase) to afford (2R)-1-((R)-2-amino-3-methylbutanoyl)-N-(1,1,1-trifluoro-2-hydroxy-4-methylpentan-3-yl)pyrrolidine-2-carboxamide (1g) as a sticky gum after evaporation/lyophilization. ¹H NMR (400 MHz, MeOD) δ 0.89-0.92 (m, 6H), 1.00 (d, J=6.9 Hz, 3H), 1.11 (d, J=7.0 Hz, 3H), 1.85-1.96 (m, 2H), 2.09-2.31 (m, 5H), 3.57-3.73 (m, 2H), 3.83-3.88 (m, 1H), 4.05 (d, J=4.8 Hz, 1H), 4.12-4.17 (m, 1H), 4.45 (t, J=6.3 Hz, 1H), 8.04 (d, J=10.1 Hz, 1H); MS: m/z 368 (M+H).

Compound 1d (50 mg, 0.068 mmol), compound 1g (51 mg, 0.136 mmol), PyBOP (71 mg, 0.136 mmol) and DIEA (44 mg, 0.341 mmol) were dissolved in anhydrous DMF (10 mL) and stirred for 3 hours. The crude product mixture was evaporated under vacuum and purified by Prep HPLC using a 20-90% linear gradient of acetonitrile in water gradient (0.05% TFA modified mobile phase) to afford compound 1h as a white solid after evaporation/lyophilization (20 mg, 21% yield).

Compound 1h was then oxidized using Dess-Martin periodinane (23 mg, 0.054 mmol) and purified by Prep HPLC to afford compound 1 as a white solid (3.5 mg, 17% yield). ¹H NMR (400 MHz, MeOD) δ 0.85-1.12 (m, 12H), 1.30 (s, 1H), 1.48 (s, 1H), 1.87-2.26 (m, 10H), 3.11-3.18 (m, 2H), 3.28 (t, J=5.2 Hz, 3H), 3.50-3.79 (m, 12H), 4.06-4.10 (m, 1H), 4.19-4.26 (m, 1H), 4.56-4.58 (m, 1H), 4.65-4.69 (m, 1H), 7.91-7.96 (m, 1H), 7.62-7.66 (m, 4H); MS: m/z 1427 (M+H).

### Example 2

### Synthesis of Compound 2

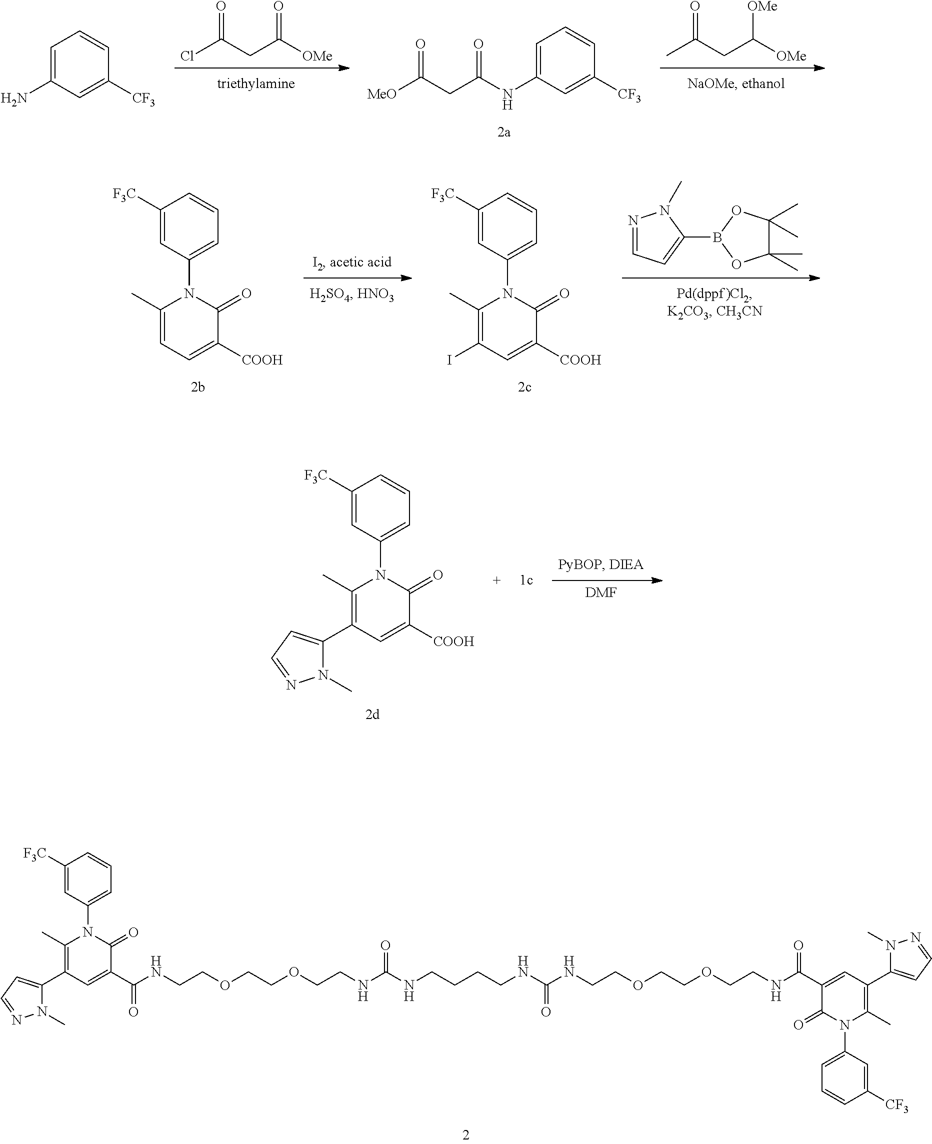

To a solution of 3-(trifluoromethyl)aniline (6.5 g, 40.3 mmol) and triethylamine (3.71 g, 36.7 mmol) in acetone (50 mL) was added methyl malonyl chloride (5 g, 36.7 mmol) dropwise at 0° C. This reaction mixture was allowed to stir for 2 hours then concentrated under vacuum and crude viscous liquid was purified by column chromatography using 10% ethyl acetate in hexenes to afford methyl 3-oxo-3-(3-(trifluoromethyl)phenylamino)propanoate (2a) (7.5 g, 71% yield). ¹H NMR (400 MHz, CDCl₃) δ 3.53 (s, 3H); 3.84 (s, 3H); 7.39-7.49 (m, 2H), 7.78 (d, J=7.9 Hz, 1H), 7.88 (s, 1H), 9.46 (s, 1H). MS: m/z 262 (M+H).

To a solution of methyl 3-oxo-3-(3-(trifluoromethyl)phenylamino)propanoate (Compound 2a) (7.5 g, 28.7 mmol) and 4,4-dimethoxybutan-2-one (4.96 g, 37.5 mmol) in ethanol (50 mL) was added sodium methoxide (3.16 g, 58.5 mmol). This reaction mixture was heated at 55° C., for 2.5 hours then cooled and 1N aq NaOH was added and stirred at rt for 1 hour. After that, ethanol was removed under vacuum and extracted with ether (2×30 mL). The aqueous layer was separated and added 1N HCl to precipitate 6-methyl-2-oxo-1-[3-(trifluoromethyl)phenyl]-1,2-dihydropyridine-3-carboxylic acid (2b) as a yellow solid, which was filtered and dried to afford 4.9 g (57% yield). ¹H NMR (400 MHz, CDCl₃) δ 2.17 (s, 3H); 6.59 (d, J=7.4 Hz, 1H), 7.49 (d, J=7.8 Hz, 1H), 7.56 (s, 1H), 7.77-7.88 (m, 2H), 8.55 (d, J=7.4 Hz, 1H), 13.72 (brs, 1H). MS: m/z 298 (M+H).

To a mixture of 6-methyl-2-oxo-1-[3-(trifluoromethyl)phenyl]-1,2-dihydropyridine-3-carboxylic acid (2b) (1.6 g, 5.4 mmol), acetic acid (15 mL) and iodine (681 mg, 2.6 mmol) was added H₂SO₄ (1 mL). This reaction mixture was then heated at 55° C. and added HNO₃ (1 mL) drop wise and continued heating for 2 more hours. The progress of the reaction was monitored by LCMS. After completion of the reaction, the crude was poured into cold water and filtered to afford dark yellow solid which was then washed with cold acetone to get 5-iodo-6-methyl-2-oxo-1-[3-(trifluoromethyl)phenyl]-1,2-dihydropyridine-3-carboxylic acid (2c) as a bright yellow solid (1 gram, 43% yield). ¹H NMR (400 MHz, CDCl₃) δ 3.84 (s, 3H); 7.45 (d, J=7.8 Hz, 1H), 7.52 (s, 1H), 7.80-7.89 (m, 2H), 8.89 (s, 1H), 13.49 (brs, 1H). MS: m/z 423.9 (M+H).

A mixture of 5-iodo-6-methyl-2-oxo-1-[3-(trifluoromethyl)phenyl]-1,2-dihydropyridine-3-carboxylic acid (2c) (500 mg, 1.18 mmol), 1-methyl-5-(4,4,5,5-tetramethyl-1,3,2-dioxaborolan-2-yl)-1H-pyrazole (491 mg, 2.36 mmol), (1,1′-(di-tert-butylphosphino)ferrocene)palladium(II) dichloride (39 mg, 0.059 mmol) and K₂CO₃ (490 mg, 3.5 mmol) was taken in a sealed tube, which was then added anhydrous acetonitrile (10 mL) and triethyl amine (1 mL). This reaction tube was degased and bubbled with argon three times and heated under microwave irradiation at 90° C. for 10 minutes. The crude reaction mixture was concentrated and purified by column chromatography using 5% methanol in dichloromethane to afford compound 2d (230 mg, 52% yield). ¹H NMR (400 MHz, CDCl₃) δ 2.03 (s, 3H); 3.80 (s, 3H); 6.31 (s, 1H), 7.53-7.61 (m, 3H), 7.83-7.91 (m, 2H), 8.51 (s, 1H), 13.49 (s, 1H); MS: m/z 378 (M+H).

6-methyl-5-(1-methyl-1H-pyrazol-5-yl)-2-oxo-1-[3-(trifluoromethyl)phenyl]-1,2-dihydropyridine-3-carboxylic acid (2d) (75 mg, 0.19 mmol), 1,1′-(butane-1,4-diyl)bis(3-(2-(2-(2-aminoethoxy)ethoxy)ethyl)urea) (1c) (50 mg, 0.098 mmol), PyBOP (101 mg, 0.19 mmol) and DIEA (80 mg, 0.62 mmol) were dissolved in anhydrous DMF (10 mL) and stirred for 3 hours. The crude product mixture was evaporated under vacuum and purified by Prep HPLC using a 30-95% linear gradient of acetonitrile in water gradient (0.05% TFA modified mobile phase) to afford final compound as a light brown solid after evaporation/lyophilization, 30 mg, 13% yield. ¹H NMR (400 MHz, MeOD) δ 1.33 (s, 2H), 1.82 (s, 3H), 2.95 (s, 2H), 3.05 (s, 2H), 3.33 (s, 2H), 3.47-3.51 (m, 8H), 3.67 (m, 3H), 6.28 (s, 1H), 7.47 (s, 1H), 7.57-7.59 (m, 1H), 7.72-7.77 (m, 3H), 8.25 (s, 1H); MS: m/z 1155 (M+H).

The compounds in Examples 3-20 were prepared using analogous procedures as described in Examples 1 and 2.

### Example 3

### Synthesis of Compound 3

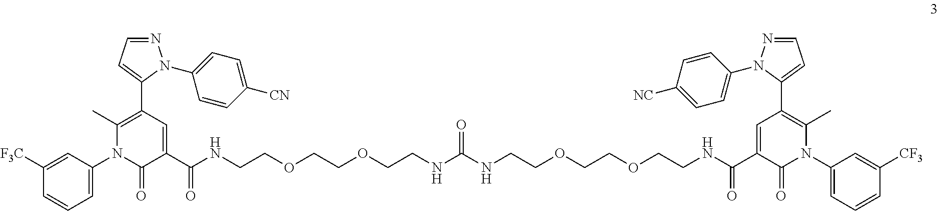 [SMILES: Cc1c(-c2ccnn2-c2ccc(C#N)cc2)cc(C(=O)NCCOCCOCCNC(=O)NCCOCCOCCNC(=O)c2cc(-c3ccnn3-c3ccc(C#N)cc3)c(C)n(-c3cccc(C(F)(F)F)c3)c2=O)c(=O)n1-c1cccc(C(F)(F)F)c1]

Characterization data: ¹H NMR (400 MHz, CDCl₃+MeOD) δ 1.62 (s, 3H), 3.09 (s, 2H), 3.29 (s, 6H), 3.39 (s, 2H), 6.50 (s, 1H), 7.35-7.44 (m, 3H), 7.65-7.74 (m, 5H), 8.34 (s, 1H); MS: m/z 1215 (M+H).

### Example 4

### Synthesis of Compound 4

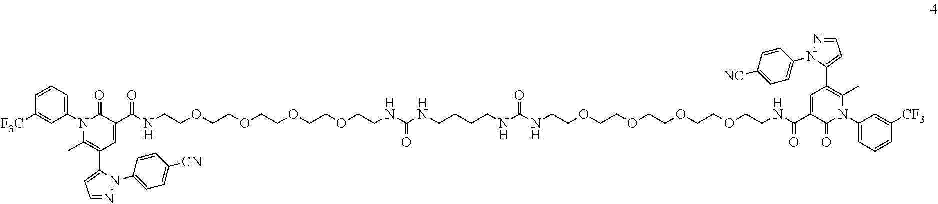 [SMILES: Cc1c(-c2ccnn2-c2ccc(C#N)cc2)cc(C(=O)NCCOCCOCCOCCOCCNC(=O)NCCCCNC(=O)NCCOCCOCCOCCOCCNC(=O)c2cc(-c3ccnn3-c3ccc(C#N)cc3)c(C)n(-c3cccc(C(F)(F)F)c3)c2=O)c(=O)n1-c1cccc(C(F)(F)F)c1]

Characterization data: ¹H NMR (400 MHz, MeOD) δ 1.45 (s, 2H), 1.83 (s, 3H), 3.09 (s, 2H), 3.24-3.27 (m, 2H), 3.47-3.59 (m, 18H), 6.69 (s, 1H), 7.57-7.63 (m, 3H), 7.71-7.87 (m, 6H), 8.22 (s, 1H); MS: m/z 1505 (M+H).

### Example 5

### Synthesis of Compound 5

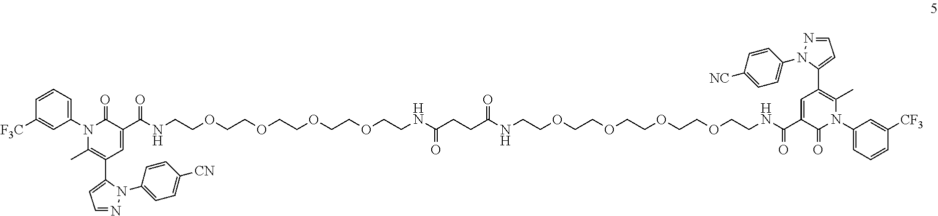 [SMILES: Cc1c(-c2ccnn2-c2ccc(C#N)cc2)cc(C(=O)NCCOCCOCCOCCOCCNC(=O)CCC(=O)NCCOCCOCCOCCOCCNC(=O)c2cc(-c3ccnn3-c3ccc(C#N)cc3)c(C)n(-c3cccc(C(F)(F)F)c3)c2=O)c(=O)n1-c1cccc(C(F)(F)F)c1]

Characterization data: ¹H NMR (400 MHz, MeOD) δ 1.73 (s, 3H), 2.36 (s, 2H), 3.38-3.49 (m, 20H), 6.60 (s, 1H), 7.47-7.53 (m, 3H), 7.61-7.78 (m, 6H), 8.13 (s, 1H); MS: m/z 1447 (M+H).

### Example 6

### Synthesis of Compound 6

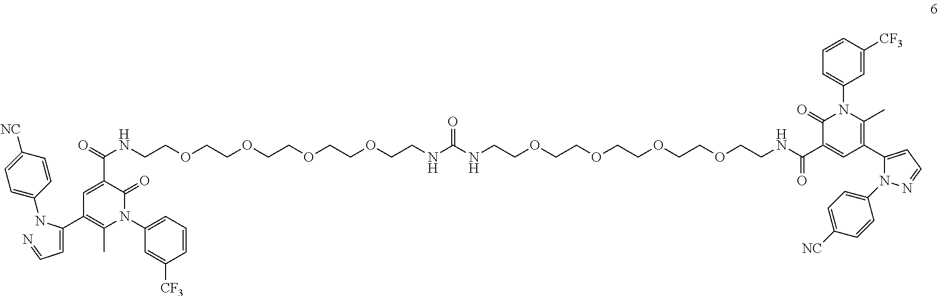 [SMILES: Cc1c(/C(=C/C=N)Nc2ccc(C#N)cc2)cc(C(=O)NCCOCCOCCOCCOCCNC(=O)NCCOCCOCCOCCOCCNC(=O)c2cc(-c3ccnn3-c3ccc(C#N)cc3)c(C)n(-c3cccc(C(F)(F)F)c3)c2=O)c(=O)n1-c1cccc(C(F)(F)F)c1]

Characterization data: ¹H NMR (400 MHz, MeOD) 1.83 (s, 3H), 3.26-3.28 (m, 2H), 3.47-3.60 (m, 18H), 6.70 (s, 1H), 7.57-7.64 (m, 3H), 7.72-7.88 (m, 6H), 8.23 (s, 1H); MS: m/z 1391 (M+H).

### Example 7

### Synthesis of Compound 7

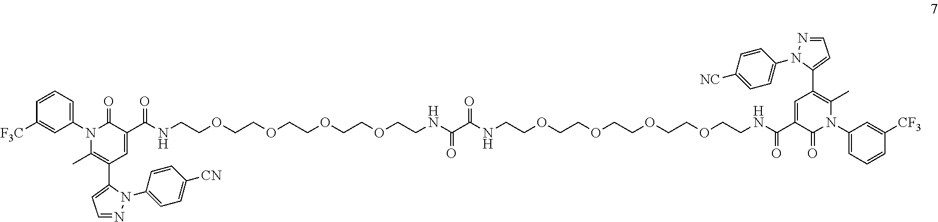 [SMILES: Cc1c(-c2ccnn2-c2ccc(C#N)cc2)cc(C(=O)NCCOCCOCCOCCOCCNC(=O)C(=O)NCCOCCOCCOCCOCCNC(=O)c2cc(-c3ccnn3-c3ccc(C#N)cc3)c(C)n(-c3cccc(C(F)(F)F)c3)c2=O)c(=O)n1-c1cccc(C(F)(F)F)c1]

Characterization data: ¹H NMR (400 MHz, MeOD) δ 1.82 (s, 3H), 3.40-3.41 (m, 2H), 3.53-3.59 (m, 18H), 6.70 (s, 1H), 7.57-7.64 (m, 3H), 7.72-7.88 (m, 6H), 8.25 (s, 1H); MS: m/z 1419 (M+H).

### Example 8

### Synthesis of Compound 8

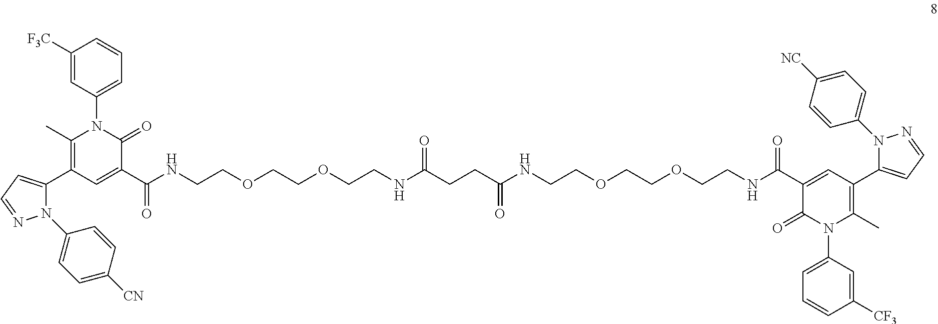 [SMILES: Cc1c(-c2ccnn2-c2ccc(C#N)cc2)cc(C(=O)NCCOCCOCCNC(=O)CCC(=O)NCCOCCOCCNC(=O)c2cc(-c3ccnn3-c3ccc(C#N)cc3)c(C)n(-c3cccc(C(F)(F)F)c3)c2=O)c(=O)n1-c1cccc(C(F)(F)F)c1]

Characterization data: ¹H NMR (400 MHz, MeOD) δ 1.84 (s, 3H), 2.39 (s, 2H), 3.19-3.20 (m, 2H), 3.45 (t, J=5.3 Hz, 2H), 3.54-3.60 (m, 8H), 6.71 (s, 1H), 7.59-7.65 (m, 3H), 7.75-7.88 (m, 6H), 8.24 (s, 1H); MS: m/z 1271 (M+H).

### Example 9

### Synthesis of Compound 9

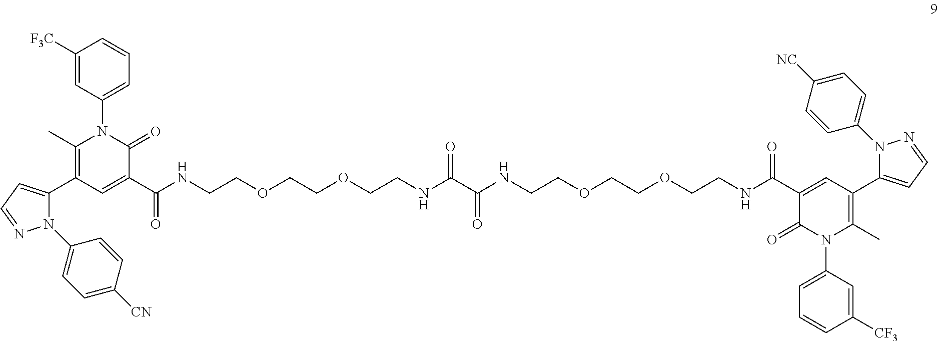 [SMILES: Cc1c(-c2ccnn2-c2ccc(C#N)cc2)cc(C(=O)NCCOCCOCCNC(=O)C(=O)NCCOCCOCCNC(=O)c2cc(-c3ccnn3-c3ccc(C#N)cc3)c(C)n(-c3cccc(C(F)(F)F)c3)c2=O)c(=O)n1-c1cccc(C(F)(F)F)c1]

Characterization data: ¹H NMR (400 MHz, MeOD) δ 1.81 (s, 3H), 3.20-3.21 (m, 2H), 3.49-3.61 (m, 10H), 6.71 (s, 1H), 7.55-7.65 (m, 3H), 7.71-7.88 (m, 5H), 8.27-8.29 (s, 2H); MS: m/z 1243 (M+H).

### Example 10

### Synthesis of Compound 10

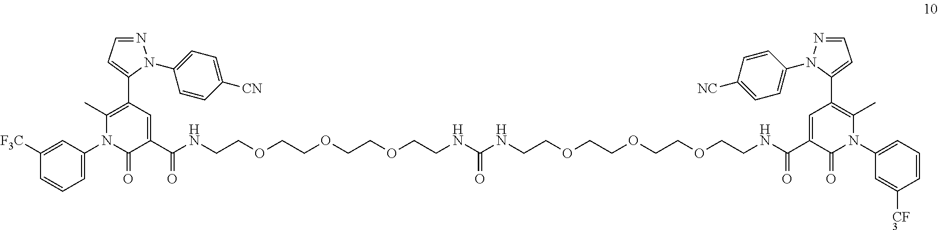 [SMILES: Cc1c(-c2ccnn2-c2ccc(C#N)cc2)cc(C(=O)NCCOCCOCCOCCNC(=O)NCCOCCOCCOCCNC(=O)c2cc(-c3ccnn3-c3ccc(C#N)cc3)c(C)n(-c3cccc(C(F)(F)F)c3)c2=O)c(=O)n1-c1cccc(C(F)(F)F)c1]

Characterization data: ¹H NMR (400 MHz, MeOD) δ 1.82 (s, 3H), 3.21-3.24 (m, 2H), 3.43-3.59 (m 14H), 6.69 (s, 1H), 7.58-7.64 (m, 3H), 7.73-7.88 (m, 6H), 8.22 (s, 1H); MS: m/z 1303 (M+H).

### Example 11

### Synthesis of Compound 11

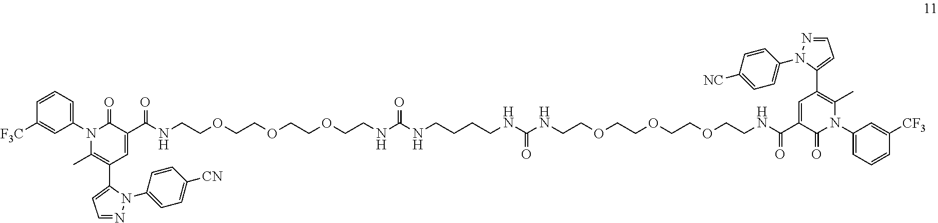 [SMILES: Cc1c(-c2ccnn2-c2ccc(C#N)cc2)cc(C(=O)NCCOCCOCCOCCNC(=O)NCCCCNC(=O)NCCOCCOCCOCCNC(=O)c2cc(-c3ccnn3-c3ccc(C#N)cc3)c(C)n(-c3cccc(C(F)(F)F)c3)c2=O)c(=O)n1-c1cccc(C(F)(F)F)c1]

Characterization data: ¹H NMR (400 MHz, MeOD) δ 1.45 (s, 2H), 1.81 (s, 3H), 3.09 (s, 2H), 3.22-3.24 (m, 2H), 3.43-3.61 (m, 14H), 6.70 (s, 1H), 7.58-7.64 (m, 3H), 7.74-7.88 (m, 6H), 8.23 (s, 1H); MS: m/z 1417 (M+H).

### Example 12

### Synthesis of Compound 12

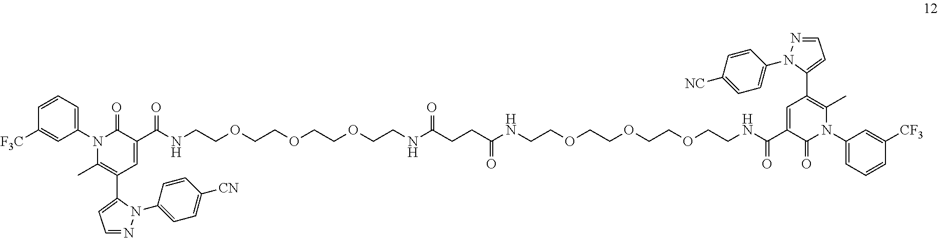 [SMILES: Cc1c(-c2ccnn2-c2ccc(C#N)cc2)cc(C(=O)NCCOCCOCCOCCNC(=O)CCC(=O)NCCOCCOCCOCCNC(=O)c2cc(-c3ccnn3-c3ccc(C#N)cc3)c(C)n(-c3cccc(C(F)(F)F)c3)c2=O)c(=O)n1-c1cccc(C(F)(F)F)c1]

Characterization data: ¹H NMR (400 MHz, MeOD) δ 1.81 (s, 3H), 2.42 (s, 2H), 3.24-3.29 (m, 2H), 3.41-3.58 (m, 14H), 6.68 (s, 1H), 7.55-7.62 (m, 3H), 7.70-7.85 (m, 6H), 8.20 (s, 1H); MS: m/z 1359 (M+H).

### Example 13

### Synthesis of Compound 13

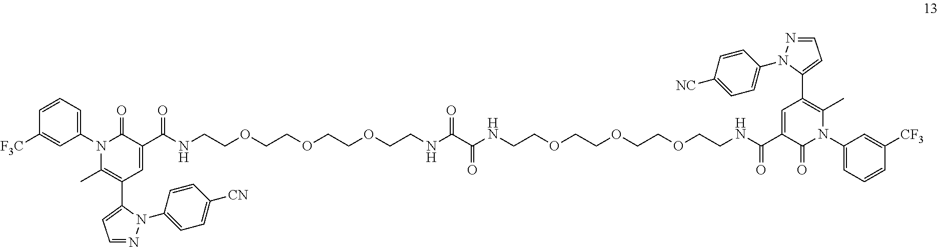 [SMILES: Cc1c(-c2ccnn2-c2ccc(C#N)cc2)cc(C(=O)NCCOCCOCCOCCNC(=O)C(=O)NCCOCCOCCOCCNC(=O)c2cc(-c3ccnn3-c3ccc(C#N)cc3)c(C)n(-c3cccc(C(F)(F)F)c3)c2=O)c(=O)n1-c1cccc(C(F)(F)F)c1]

Characterization data: ¹H NMR (400 MHz, MeOD) δ 1.81 (s, 3H), 3.31-3.37 (m, 2H), 3.48-3.59 (m, 14H), 6.69 (s, 1H), 7.56-7.64 (m, 3H), 7.72-7.87 (m, 6H), 8.24 (s, 1H); MS: m/z 1331 (M+H).

### Example 14

### Synthesis of Compound 14

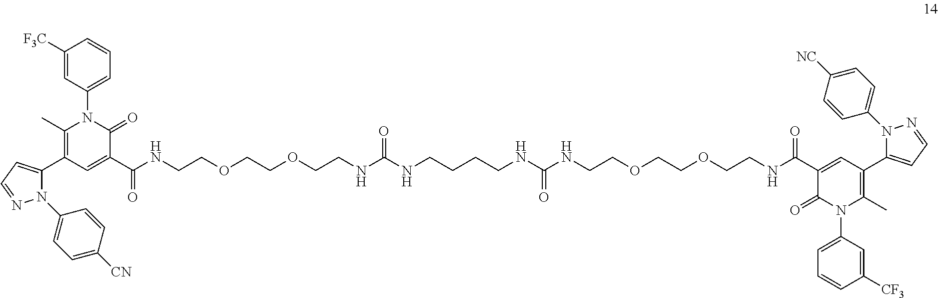 [SMILES: Cc1c(-c2ccnn2-c2ccc(C#N)cc2)cc(C(=O)NCCOCCOCCNC(=O)NCCCCNC(=O)NCCOCCOCCNC(=O)c2cc(-c3ccnn3-c3ccc(C#N)cc3)c(C)n(-c3cccc(C(F)(F)F)c3)c2=O)c(=O)n1-c1cccc(C(F)(F)F)c1]

Characterization data: ¹H NMR (400 MHz, MeOD) δ 1.43 (s, 2H), 1.85 (s, 3H), 3.05 (s, 2H), 3.14 (t, J=2.7 Hz, 2H), 3.43 (t, J=5.2 Hz, 2H), 3.49-3.60 (m, 8H), 6.70 (s, 1H), 7.59-7.65 (m, 3H), 7.75-7.89 (m, 6H), 8.24 (s, 1H); MS: m/z 1329 (M+H).

### Example 15

### Synthesis of Compound 15

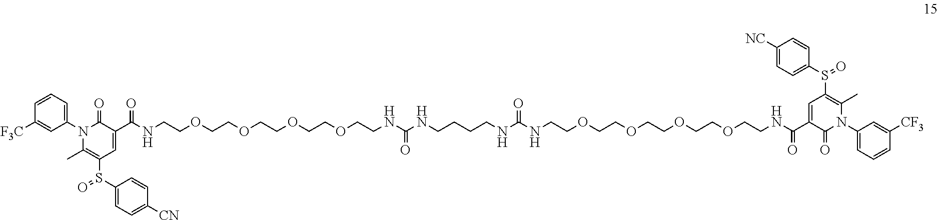 [SMILES: Cc1c(S(=O)c2ccc(C#N)cc2)cc(C(=O)NCCOCCOCCOCCOCCNC(=O)NCCCCNC(=O)NCCOCCOCCOCCOCCNC(=O)c2cc(S(=O)c3ccc(C#N)cc3)c(C)n(-c3cccc(C(F)(F)F)c3)c2=O)c(=O)n1-c1cccc(C(F)(F)F)c1]

Characterization data: ¹H NMR (400 MHz, MeOD) δ 1.45 (s, 2H), 2.44 (s, 3H), 3.09 (s, 2H), 3.25 (t, J=5.1 Hz, 2H), 3.46-3.57 (m, 18H), 7.68 (t, J=10.2 Hz, 1H), 7.84-7.87 (m, 2H), 7.91-7.97 (m, 5H), 8.50 (d, J=5.2 Hz, 1H); MS: m/z 1469 (M+H).

### Example 16

### Synthesis of Compound 16

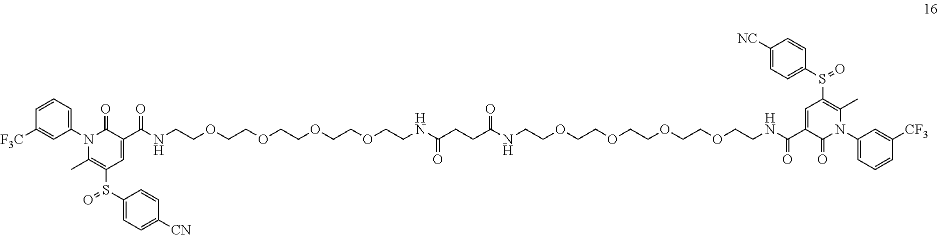 [SMILES: Cc1c(S(=O)c2ccc(C#N)cc2)cc(C(=O)NCCOCCOCCOCCOCCNC(=O)CCC(=O)NCCOCCOCCOCCOCCNC(=O)c2cc(S(=O)c3ccc(C#N)cc3)c(C)n(-c3cccc(C(F)(F)F)c3)c2=O)c(=O)n1-c1cccc(C(F)(F)F)c1]

Characterization data: ¹H NMR (400 MHz, MeOD) δ 2.45-2.48 (m, 5H), 3.50-3.58 (m, 20H), 7.69 (t, J=10.2 Hz, 1H), 7.85-7.88 (m, 2H), 7.92-7.99 (m, 5H), 8.51 (d, J=5.2 Hz, 1H); MS: m/z 1411 (M+H).

### Example 17

### Synthesis of Compound 17

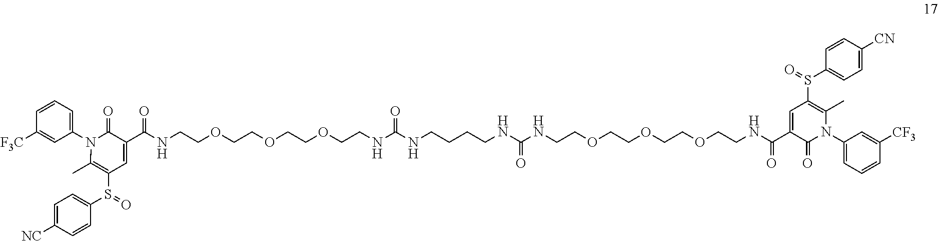 [SMILES: Cc1c(S(=O)c2ccc(C#N)cc2)cc(C(=O)NCCOCCOCCOCCNC(=O)NCCCCNC(=O)NCCOCCOCCOCCNC(=O)c2cc(S(=O)c3ccc(C#N)cc3)c(C)n(-c3cccc(C(F)(F)F)c3)c2=O)c(=O)n1-c1cccc(C(F)(F)F)c1]

Characterization data: ¹H NMR (400 MHz, MeOD) δ 1.45 (s, 2H), 2.45 (s, 3H), 3.09 (s, 2H), 3.22 (t, J=4.9 Hz, 2H), 3.41-3.57 (m, 14H), 7.68 (t, J=10.2 Hz, 1H), 7.81-7.87 (m, 2H), 7.91-7.97 (m, 5H), 8.50 (d, J=5.2 Hz, 1H); MS: m/z 1381 (M+H).

### Example 18

### Synthesis of Compound 18

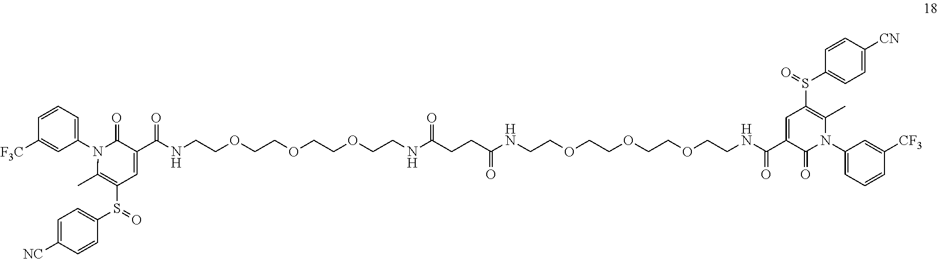 [SMILES: Cc1c(S(=O)c2ccc(C#N)cc2)cc(C(=O)NCCOCCOCCOCCNC(=O)CCC(=O)NCCOCCOCCOCCNC(=O)c2cc(S(=O)c3ccc(C#N)cc3)c(C)n(-c3cccc(C(F)(F)F)c3)c2=O)c(=O)n1-c1cccc(C(F)(F)F)c1]

Characterization data: ¹H NMR (400 MHz, MeOD) δ 2.44-2.47 (m, 5H), 3.27 (t, J=5.2 Hz, 2H), 3.41-3.57 (m, 14H), 7.65-7.68 (m, 1H), 7.80-7.86 (m, 2H), 7.90-7.96 (m, 5H), 8.50 (d, J=5.2 Hz, 1H); MS: m/z 1323 (M+H).

### Example 19

### Synthesis of Compound 19

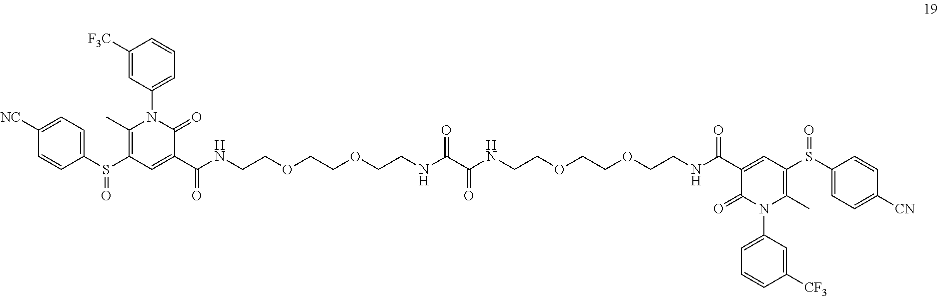 [SMILES: Cc1c(S(=O)c2ccc(C#N)cc2)cc(C(=O)NCCOCCOCCNC(=O)C(=O)NCCOCCOCCNC(=O)c2cc(S(=O)c3ccc(C#N)cc3)c(C)n(-c3cccc(C(F)(F)F)c3)c2=O)c(=O)n1-c1cccc(C(F)(F)F)c1]

Characterization data: ¹H NMR (400 MHz, MeOD) δ 2.43 (s, 3H), 3.13 (s, 2H), 3.43-3.53 (m, 10H), 7.62-7.69 (m, 1H), 7.77-7.95 (m, 7H), 8.51 (d, J=5.2 Hz, 1H); MS: m/z 1207 (M+H).

### Example 20

### Synthesis of Compound 20

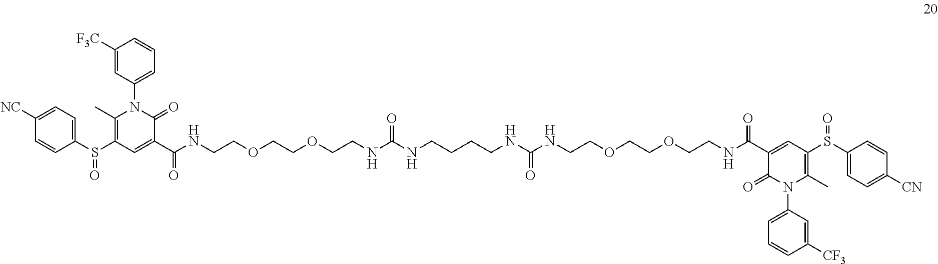 [SMILES: Cc1c(S(=O)c2ccc(C#N)cc2)cc(C(=O)NCCOCCOCCNC(=O)NCCCCNC(=O)NCCOCCOCCNC(=O)c2cc(S(=O)c3ccc(C#N)cc3)c(C)n(-c3cccc(C(F)(F)F)c3)c2=O)c(=O)n1-c1cccc(C(F)(F)F)c1]

Characterization data: ¹H NMR (400 MHz, MeOD) δ 1.39 (s, 2H), 2.42 (s, 3H), 3.02-3.08 (m, 4H), 3.37 (t, J=4.9 Hz, 2H), 3.45-3.52 (m, 8H), 7.63-7.70 (m, 1H), 7.77-7.92 (m, 7H), 8.46 (d, J=5.2 Hz, 1H), MS: m/z 1293 (M+H).

Examples 21-27 are prepared using analogous procedures as outlined in the Examples above:

| Example | Structure |
|---|---|
| 21 | 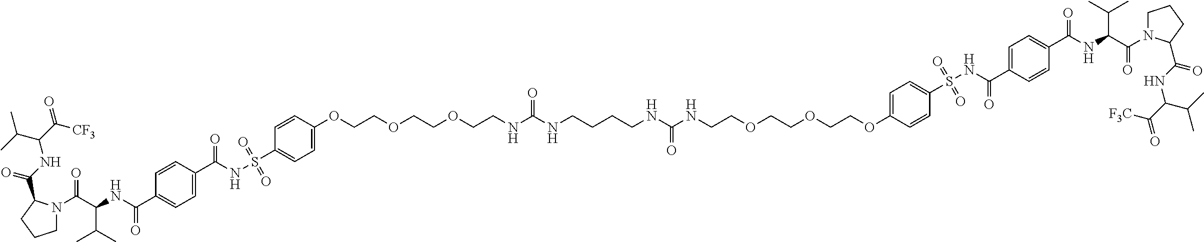 [SMILES: CC(C)C(NC(=O)C1CCCN1C(=O)[C@@H](NC(=O)c1ccc(C(=O)NS(=O)(=O)c2ccc(OCCOCCOCCNC(=O)NCCCCNC(=O)NCCOCCOCCOc3ccc(S(=O)(=O)NC(=O)c4ccc(C(=O)N[C@H](C(=O)N5CCC[C@H]5C(=O)NC(C(=O)C(F)(F)F)C(C)C)C(C)C)cc4)cc3)cc2)cc1)C(C)C)C(=O)C(F)(F)F] |
| 22 | 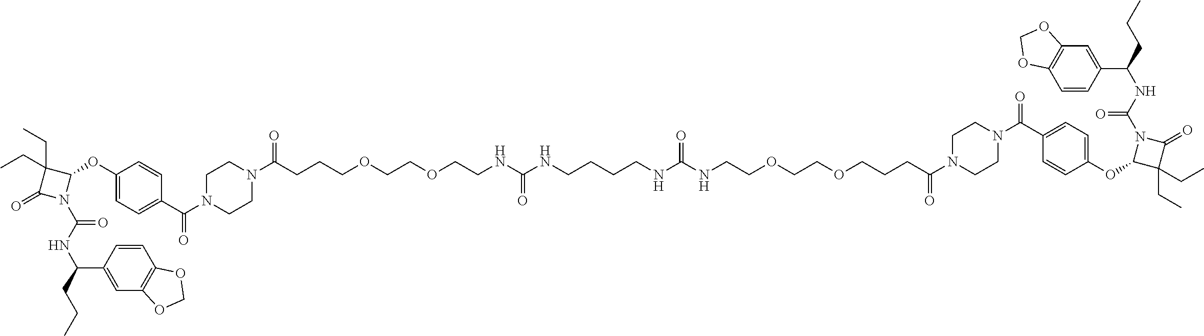 [SMILES: CCC[C@@H](NC(=O)N1C(=O)C(CC)(CC)[C@@H]1Oc1ccc(C(=O)N2CCN(C(=O)CCCOCCOCCNC(=O)NCCCCNC(=O)NCCOCCOCCCC(=O)N3CCN(C(=O)c4ccc(O[C@@H]5N(C(=O)N[C@H](CCC)c6ccc7c(c6)OCO7)C(=O)C5(CC)CC)cc4)CC3)CC2)cc1)c1ccc2c(c1)OCO2] |
| 23 | 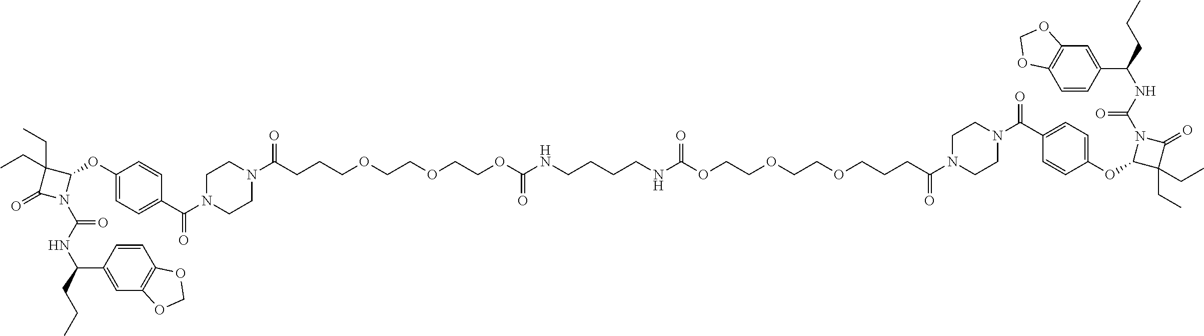 [SMILES: CCC[C@@H](NC(=O)N1C(=O)C(CC)(CC)[C@@H]1Oc1ccc(C(=O)N2CCN(C(=O)CCCOCCOCCOC(=O)NCCCCNC(=O)OCCOCCOCCCC(=O)N3CCN(C(=O)c4ccc(O[C@@H]5N(C(=O)N[C@H](CCC)c6ccc7c(c6)OCO7)C(=O)C5(CC)CC)cc4)CC3)CC2)cc1)c1ccc2c(c1)OCO2] |
| 24 | 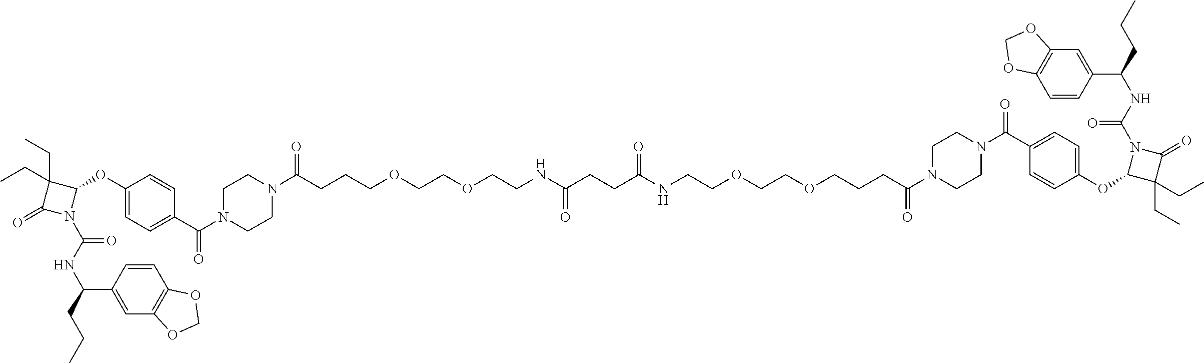 [SMILES: CCC[C@@H](NC(=O)N1C(=O)C(CC)(CC)[C@@H]1Oc1ccc(C(=O)N2CCN(C(=O)CCCOCCOCCNC(=O)CCC(=O)NCCOCCOCCCC(=O)N3CCN(C(=O)c4ccc(O[C@@H]5N(C(=O)N[C@H](CCC)c6ccc7c(c6)OCO7)C(=O)C5(CC)CC)cc4)CC3)CC2)cc1)c1ccc2c(c1)OCO2] |
| 25 | 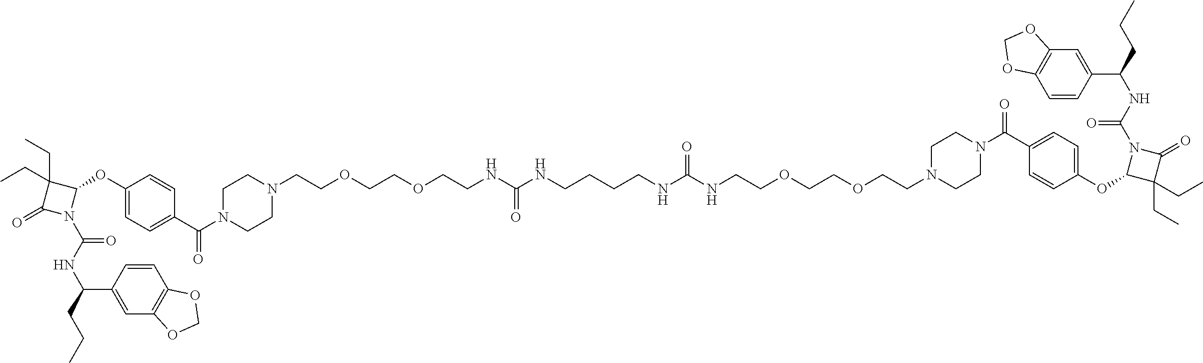 [SMILES: CCC[C@@H](NC(=O)N1C(=O)C(CC)(CC)[C@@H]1Oc1ccc(C(=O)N2CCN(CCOCCOCCNC(=O)NCCCCNC(=O)NCCOCCOCCN3CCN(C(=O)c4ccc(O[C@@H]5N(C(=O)N[C@H](CCC)c6ccc7c(c6)OCO7)C(=O)C5(CC)CC)cc4)CC3)CC2)cc1)c1ccc2c(c1)OCO2] |
| 26 | 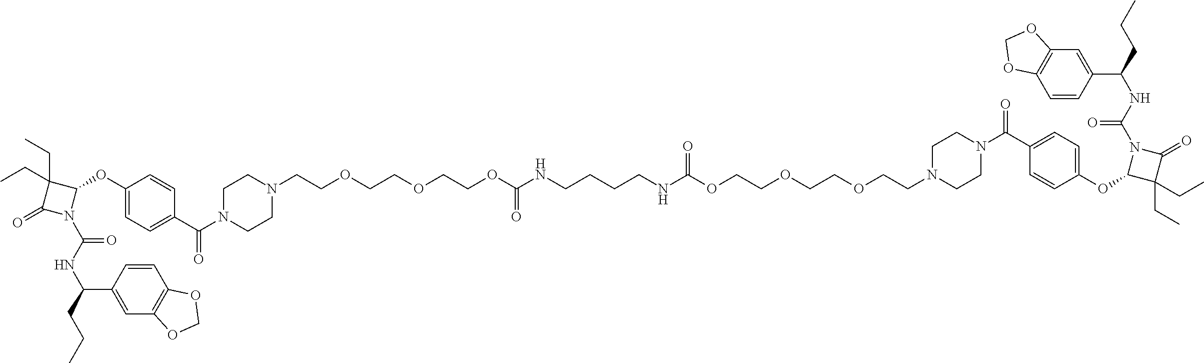 [SMILES: CCC[C@@H](NC(=O)N1C(=O)C(CC)(CC)[C@@H]1Oc1ccc(C(=O)N2CCN(CCOCCOCCOC(=O)NCCCCNC(=O)OCCOCCOCCN3CCN(C(=O)c4ccc(O[C@@H]5N(C(=O)N[C@H](CCC)c6ccc7c(c6)OCO7)C(=O)C5(CC)CC)cc4)CC3)CC2)cc1)c1ccc2c(c1)OCO2] |
| 27 | 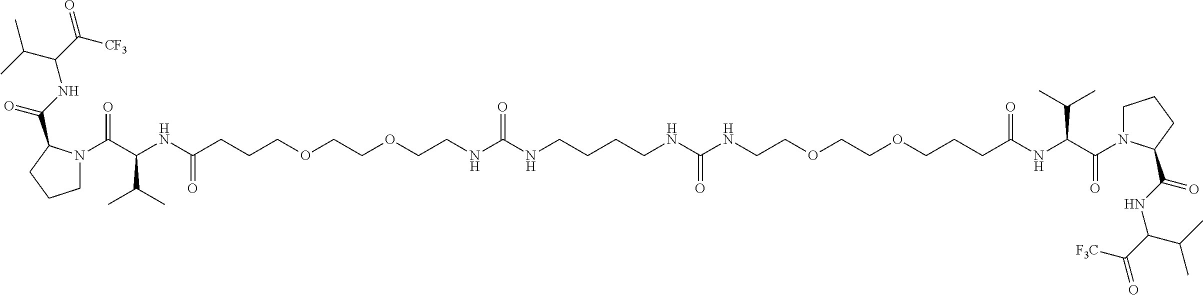 [SMILES: CC(C)C(NC(=O)[C@@H]1CCCN1C(=O)[C@@H](NC(=O)CCCOCCOCCNC(=O)NCCCCNC(=O)NCCOCCOCCCC(=O)N[C@H](C(=O)N1CCC[C@H]1C(=O)NC(C(=O)C(F)(F)F)C(C)C)C(C)C)C(C)C)C(=O)C(F)(F)F] |

### Example 28

### Elastase Enzyme In-Vitro Inhibition Assay

HNE were purchased from Elastin Products (Owensville, Mo., USA) with catalog number SE563. Fixed concentrations of HNE (20 nM) were incubated with various concentrations of inhibitors in PBS buffer in opaque 96-well plates. After 15 min incubation at room temperature, the corresponding fluorescent substrates MeOSuc-AAPV-AMC (Merck Millipore) were added to each well at final concentration 100 μM. Fluorescence was monitored on a Molecular Devices Spectramax fluorescence plate reader at 460 nm with excitation at 380 nm for 30 min. Residual enzyme activity was calculated using the slope of fluorescence increase in the first 10 min. Percentage of enzyme inhibition was plotted using GraphPad Prism 6 software (GraphPad Prism, La Jolla, Calif., USA). The IC50 value and Ki value was calculated using the Morrison tight-binding equation provided by the software.

Table 2 below shows biological activity (IC₅₀) for compounds described herein. The IC₅₀ activity is graded as: ++=<150 nM; +=150 nM to 1 μM.

TABLE 2

| Cmpd # | Activity |
|---|---|
| 1 | ++ |
| 2 | + |
| 3 | ++ |
| 4 | ++ |
| 5 | ++ |
| 6 | ++ |
| 7 | + |
| 8 | ++ |
| 9 | ++ |
| 10 | ++ |
| 11 | + |
| 12 | ++ |
| 13 | ++ |
| 14 | ++ |
| 15 | ++ |
| 16 | ++ |
| 17 | + |
| 18 | ++ |
| 19 | ++ |
| 20 | + |

1. A compound of Formula (I), a pharmaceutically acceptable salt, solvate, polymorph, prodrug, metabolite, deuteride, N-oxide, stereoisomer, or isomer thereof: 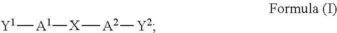
wherein:
A¹ and A² are independently selected from 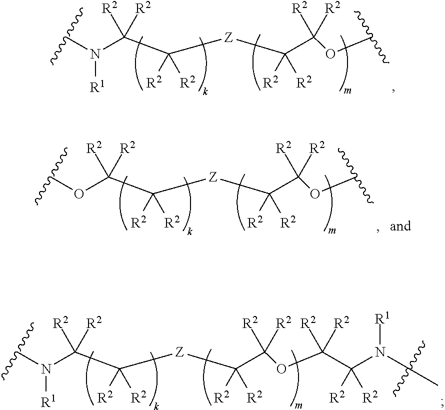
Y¹ and Y² are independently an elastase inhibitor;
X is alkylene, —C(O)—, —C(O)C(O)—, 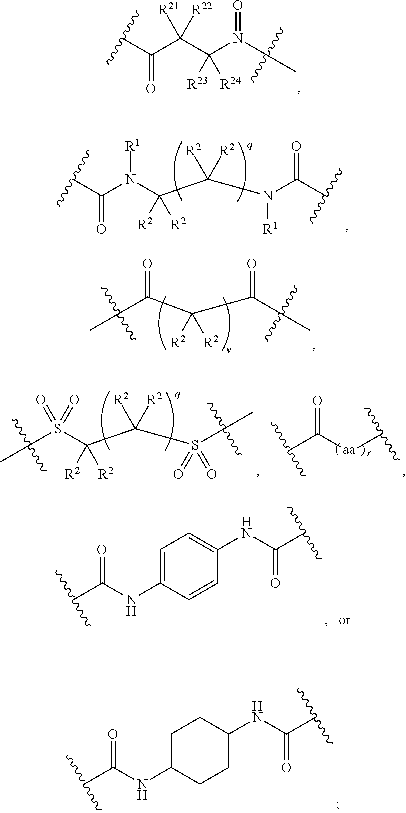
Z is O or CR²R²;
aa is 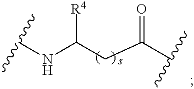
each R¹ is independently selected from H, alkyl, haloalkyl, arylalkyl, and heteroarylalkyl;
each R² is independently selected from H, halo, —CN, —SR¹, alkyl, cycloalkyl, haloalkyl, —OR¹, and —R⁴;
each R⁴ is independently selected from —CO₂R¹, -(alkylene)-(CO₂R¹), hydroxyalkyl, -(alkylene)(S(O)ₜ)(alkyl), -(alkylene)(NR⁵R⁵), and 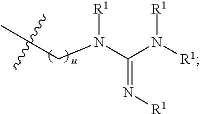
each R⁵ is independently selected from H, alkyl, haloalkyl, arylalkyl, heteroarylalkyl, aryl, and heteroaryl;
R²¹, R²², R²³, and R²⁴ are independently selected from H, halo, —CN, —SR¹, alkyl, cycloalkyl, haloalkyl, —NR¹R¹, and —OR¹;
each k is independently selected from 1, 2, 3, 4, 5, 6, 7, 8, 9, and 10;
each m is independently selected from 0, 1, 2, 3, 4, 5, 6, 7, 8, 9, and 10;
q is 1, 2, 3, 4, 5, 6, 7, 8, 9, or 10;
r is 3, 4, 5, 6, or 7;
each s is independently selected from 0, 1, 2, 3, and 4;
each t is independently selected from 0, 1, and 2;
each u is independently selected from 1, 2, 3, 4, 5, 6, 7, 8, 9, and 10; and
v is selected from 3, 4, 5, 6, 7, 8, 9, and 10.

2. The compound of claim 1 wherein Y¹ and Y² are selected from sivelestat, alvelestat, DMP-777, and a derivative thereof.

3. The compound of claim 1 wherein Y¹ and Y² are independently selected from

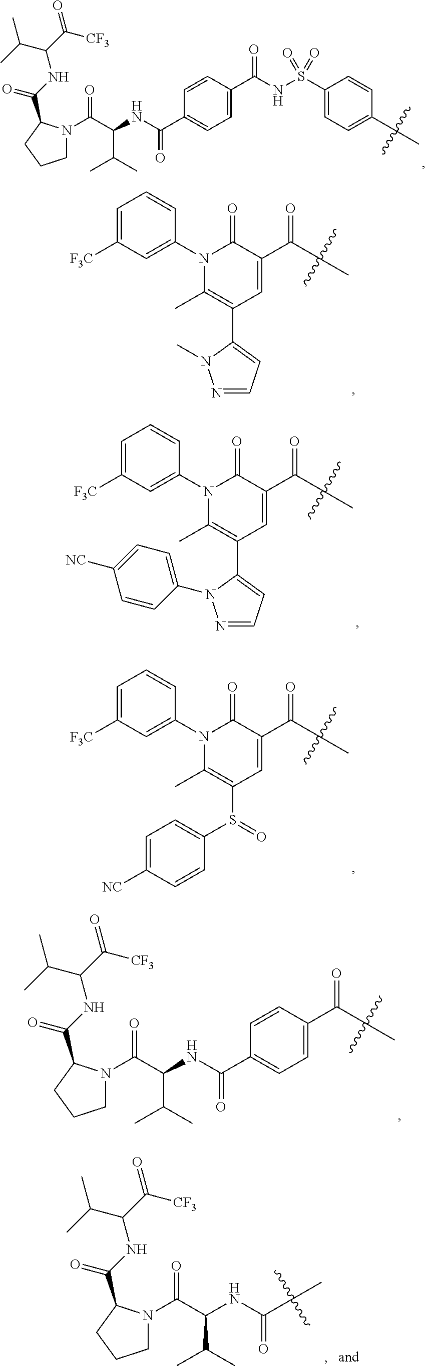

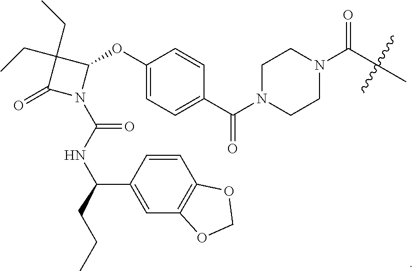 [SMILES: *C(=O)N1CCN(C(=O)c2ccc(O[C@@H]3N(C(=O)N[C@H](CCC)c4ccc5c(c4)OCO5)C(=O)C3(CC)CC)cc2)CC1]

4. The compound of claim 3 wherein Y¹ and Y² are the same.

5. The compound of claim 4 wherein Y¹ and Y² are both

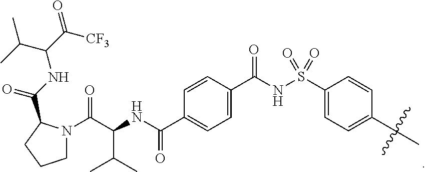 [SMILES: *c1ccc(S(=O)(=O)NC(=O)c2ccc(C(=O)N[C@H](C(=O)N3CCC[C@H]3C(=O)NC(C(=O)C(F)(F)F)C(C)C)C(C)C)cc2)cc1]

6. The compound of claim 4 wherein Y¹ and Y² are both

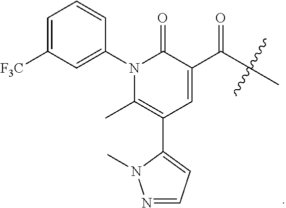 [SMILES: *C(=O)c1cc(-c2ccnn2C)c(C)n(-c2cccc(C(F)(F)F)c2)c1=O]

7. The compound of claim 4 wherein Y¹ and Y² are both

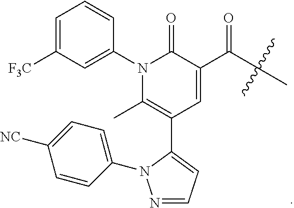 [SMILES: *C(=O)c1cc(-c2ccnn2-c2ccc(C#N)cc2)c(C)n(-c2cccc(C(F)(F)F)c2)c1=O]

8. The compound of claim 4 wherein Y¹ and Y² are both

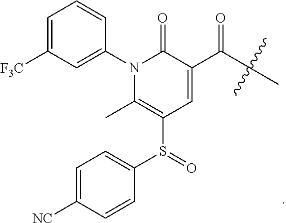 [SMILES: *C(=O)c1cc(S(=O)c2ccc(C#N)cc2)c(C)n(-c2cccc(C(F)(F)F)c2)c1=O]

9. The compound of claim 4 wherein Y¹ and Y² are both

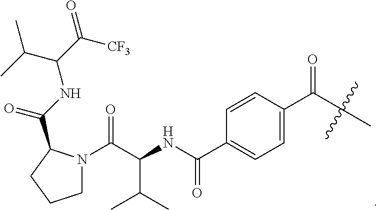 [SMILES: *C(=O)c1ccc(C(=O)N[C@H](C(=O)N2CCC[C@H]2C(=O)NC(C(=O)C(F)(F)F)C(C)C)C(C)C)cc1]

10. The compound of claim 4 wherein Y¹ and Y² are both

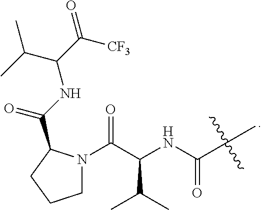 [SMILES: *C(=O)N[C@H](C(=O)N1CCC[C@H]1C(=O)NC(C(=O)C(F)(F)F)C(C)C)C(C)C]

11. The compound of claim 4 wherein Y¹ and Y² are both

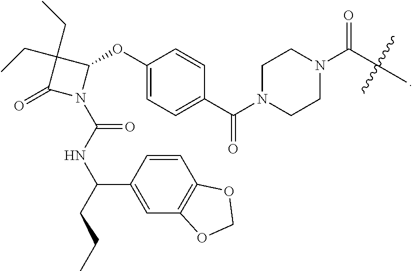

12. The compound of any one of claims 1-11 wherein A¹ and A² are the same.

13. The compound of any one of claims 1-12 wherein A¹ and A² are both

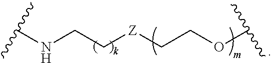

14. The compound of any one of claims 1-12 wherein A¹ and A² are both

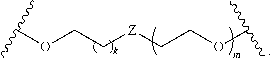

15. The compound of any one of claims 1-12 wherein A¹ and A² are both

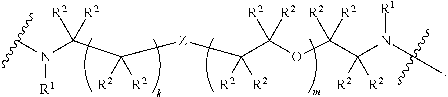

16. The compound of any one of claims 1-15 wherein Z is O.

17. The compound of any one of claims 1-16 wherein k is 1.

18. The compound of any one of claims 1-17 wherein each m is independently 0, 1, 2, or 3.

19. The compound of any one of claims 1-18 wherein X is

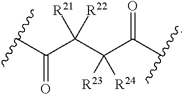

20. The compound of claim 19 wherein X is

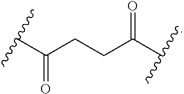 [SMILES: *C(=O)CCC(*)=O]

21. The compound of claim 19 wherein X is

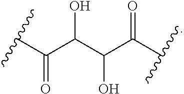 [SMILES: *C(=O)C(O)C(O)C(*)=O]

22. The compound of claim 19 wherein X is

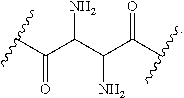 [SMILES: *C(=O)C(N)C(N)C(*)=O]

23. The compound of any one of claims 1-18 wherein X is

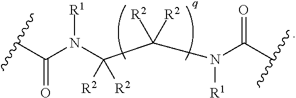

24. The compound of claim 23 wherein X is

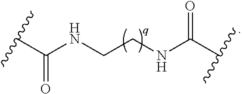

25. The compound of claim 24 wherein q is 3.

26. The compound of claim 24 wherein q is 5.

27. The compound of any one of claims 1-18 wherein X is

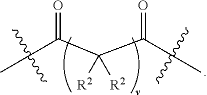

28. The compound of claim 27 wherein X is

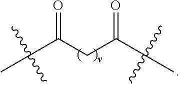

29. The compound of claim 28 wherein v is 4.

30. The compound of any one of claims 1-18 wherein X is

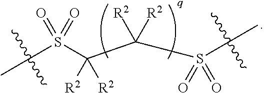

31. The compound of claim 30 wherein X is

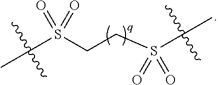

32. The compound of claim 31 wherein q is 3.

33. The compound of any one of claims 1-18 wherein X is —C(O)—.

34. The compound of any one of claims 1-18 wherein X is —C(O)C(O)—.

35. The compound of claim 1 wherein the compound is selected from:

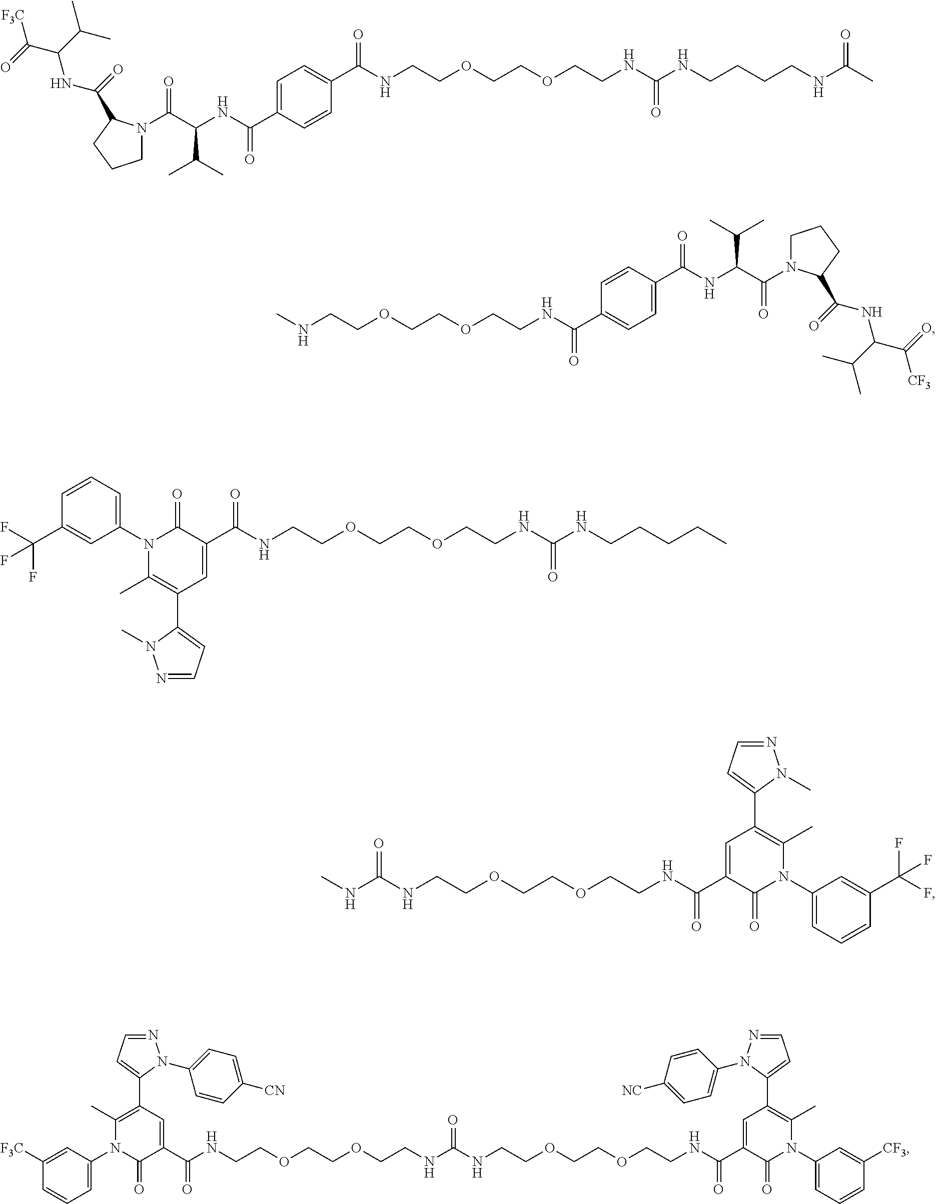

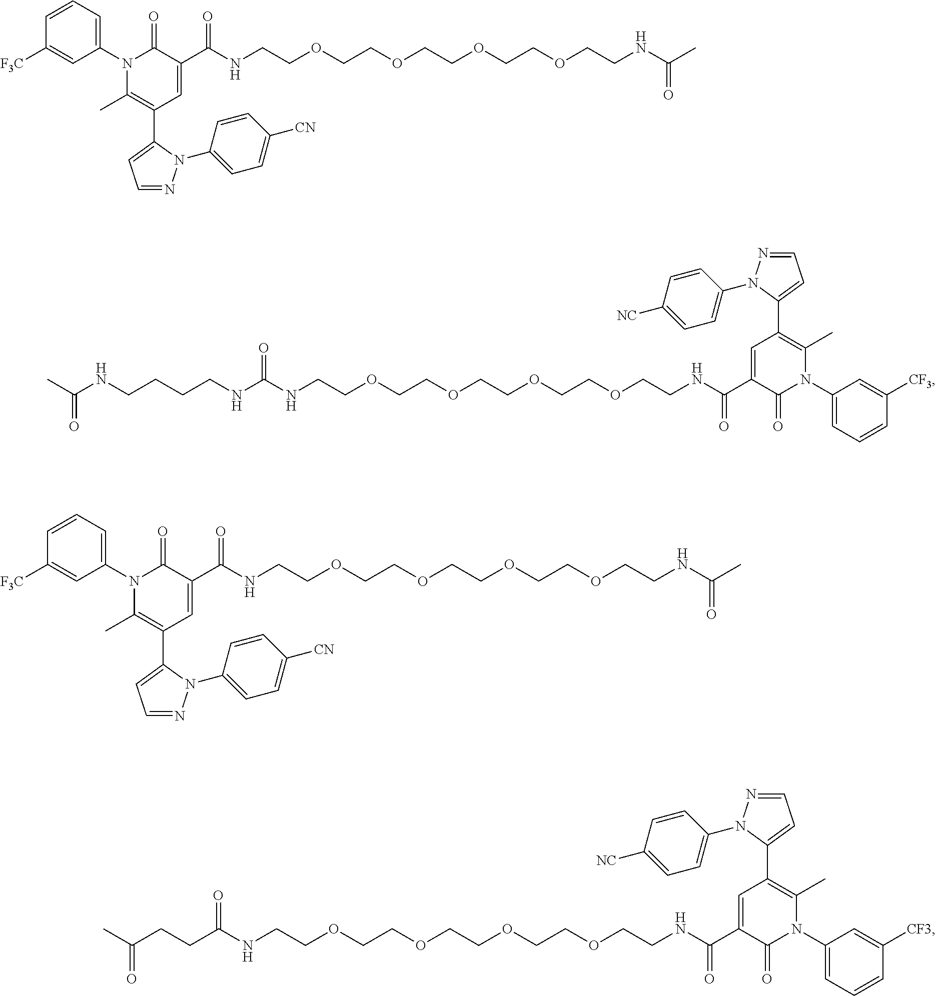

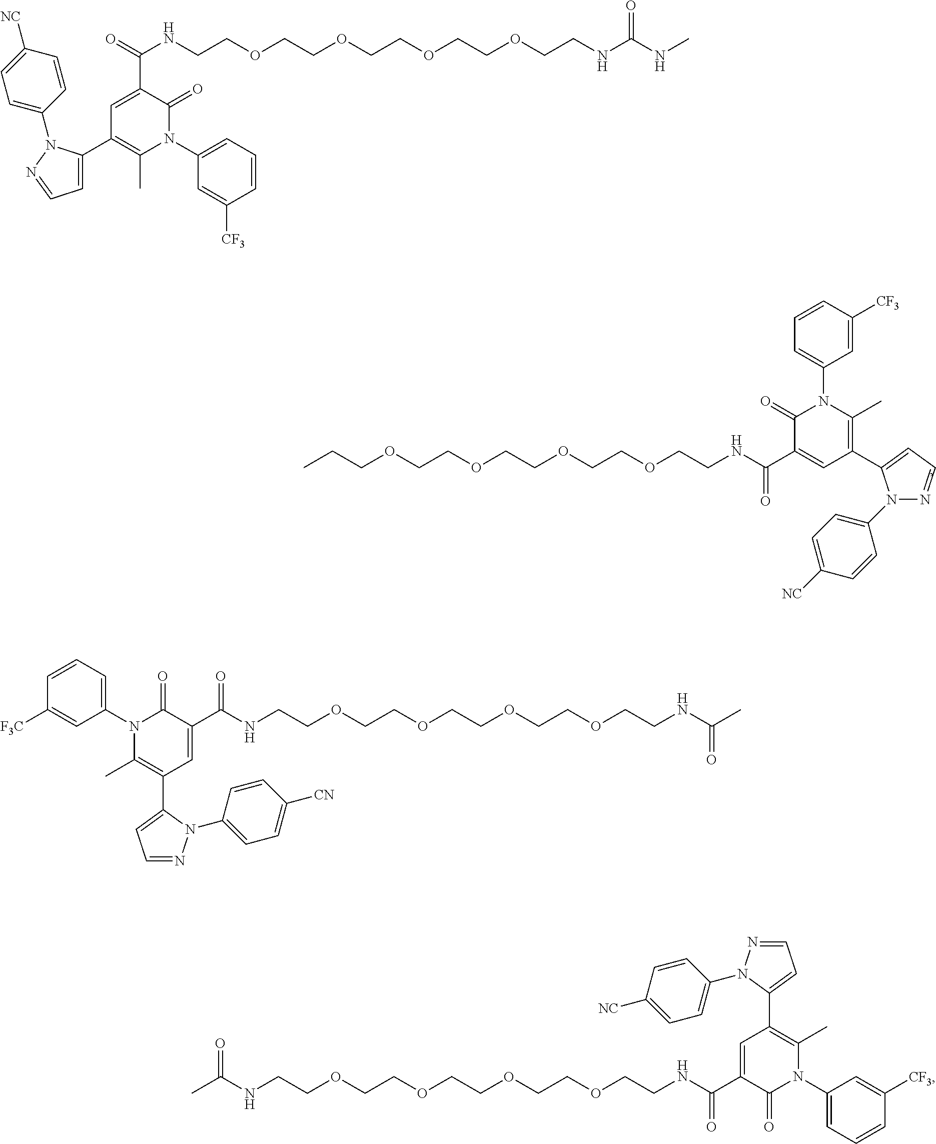

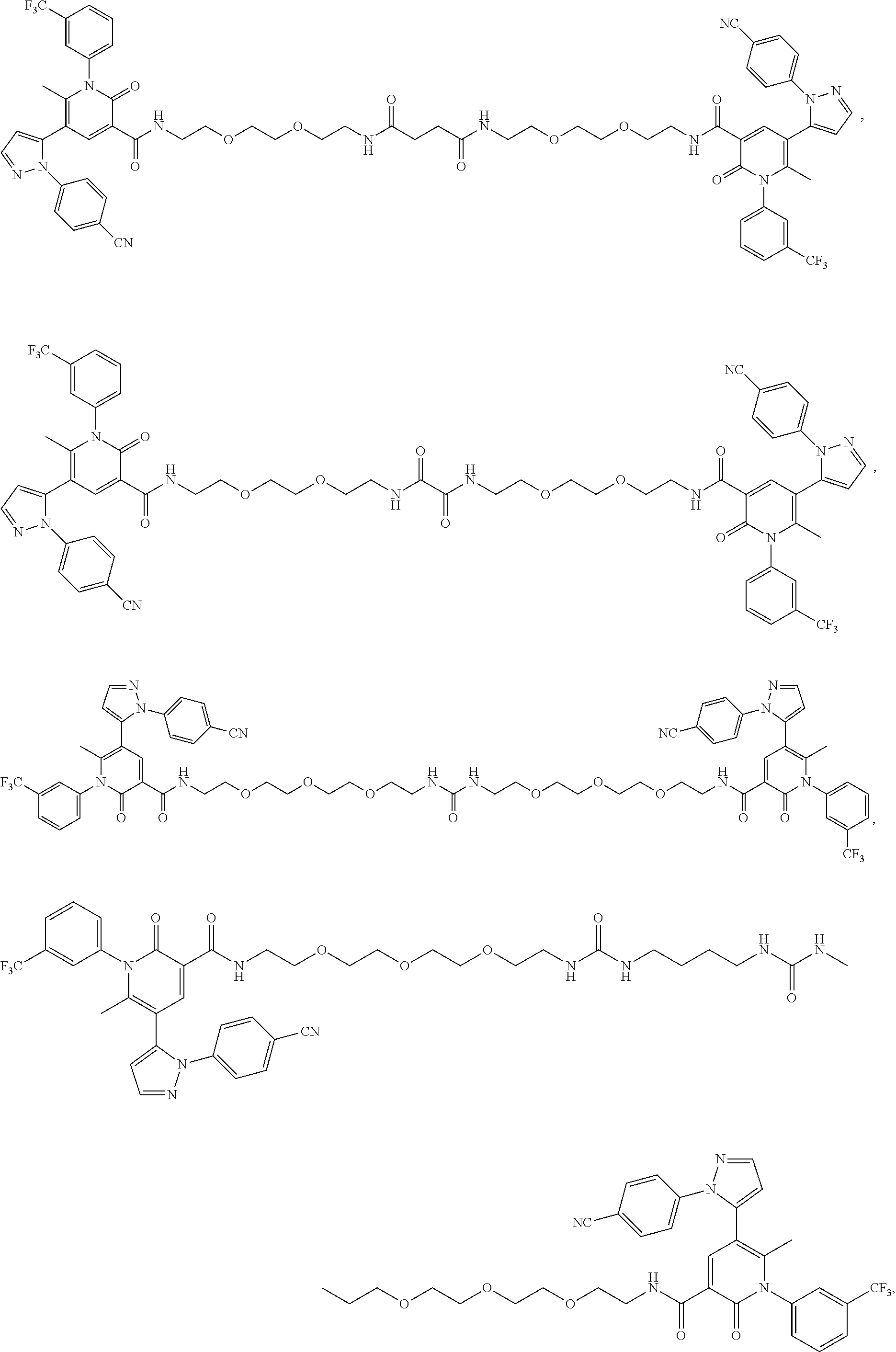

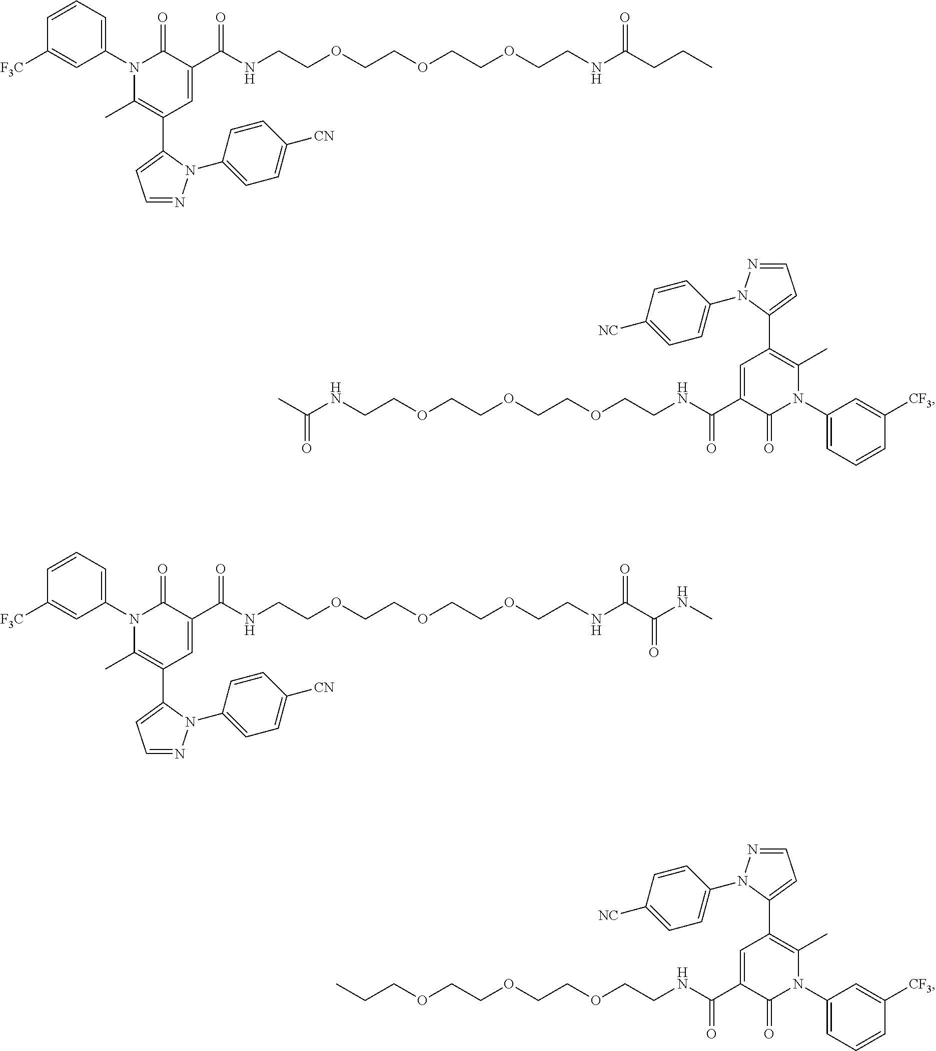

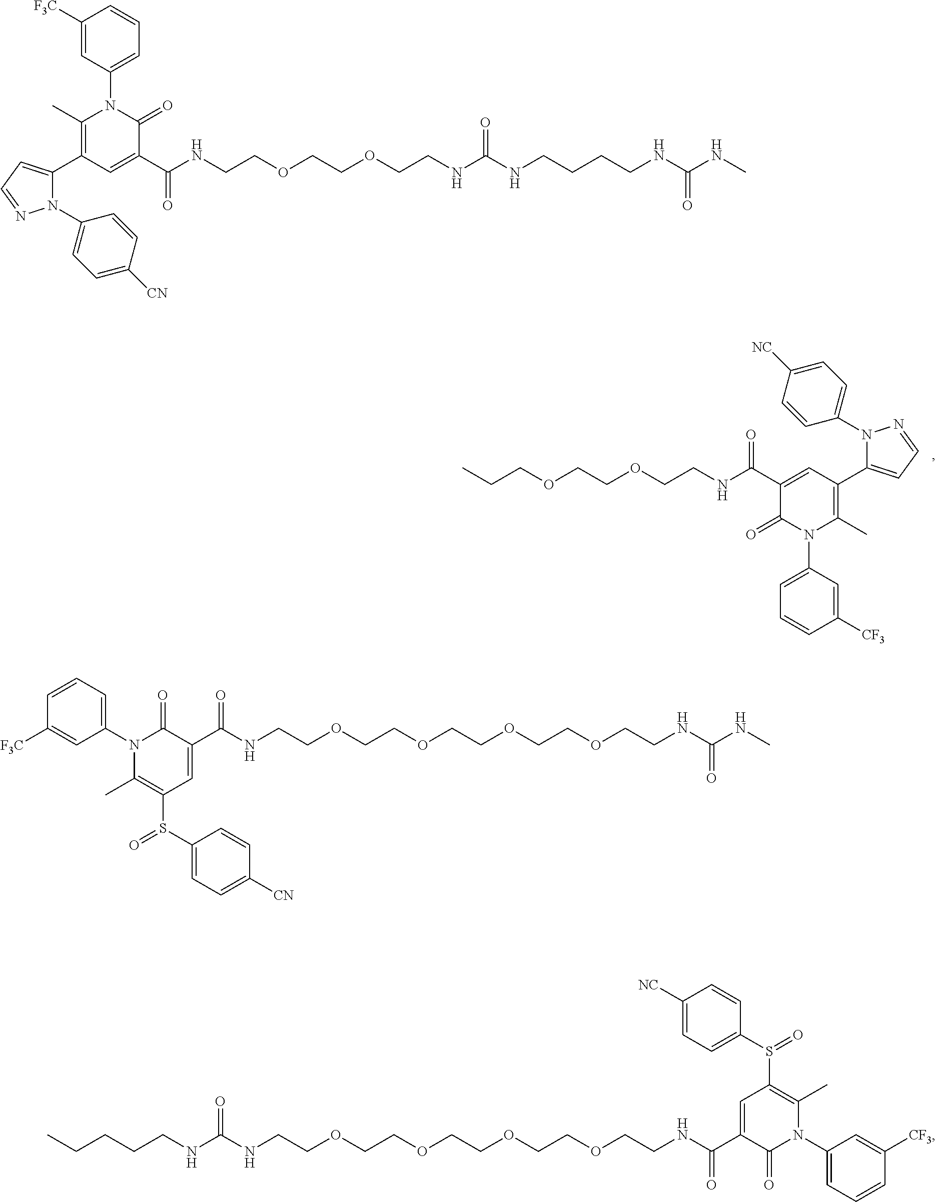

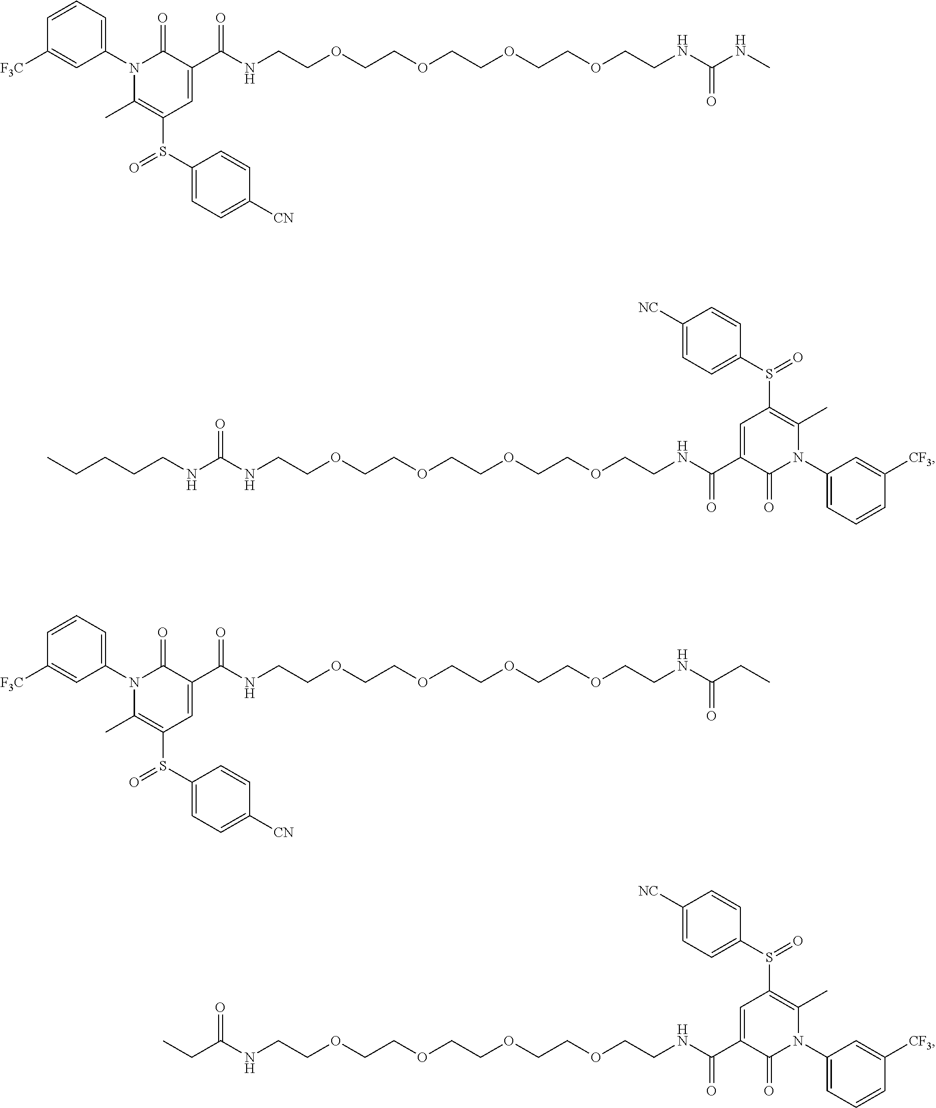

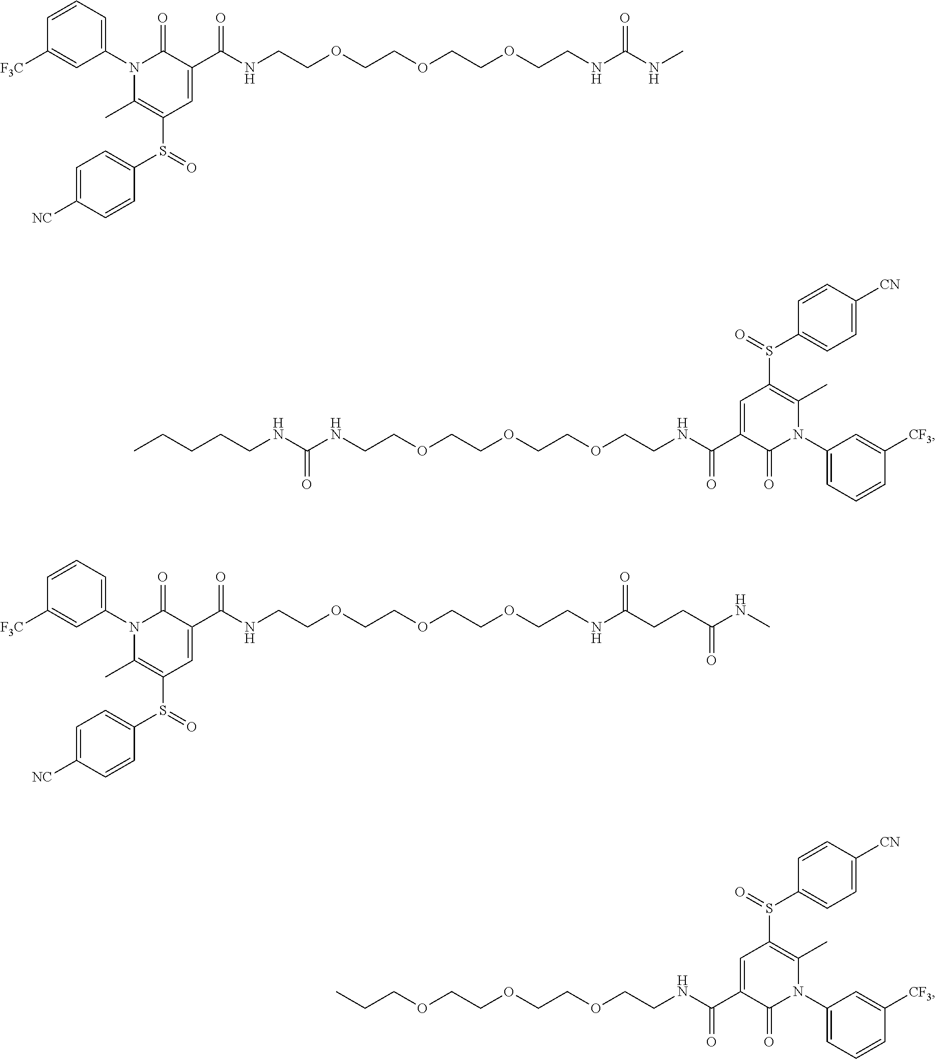

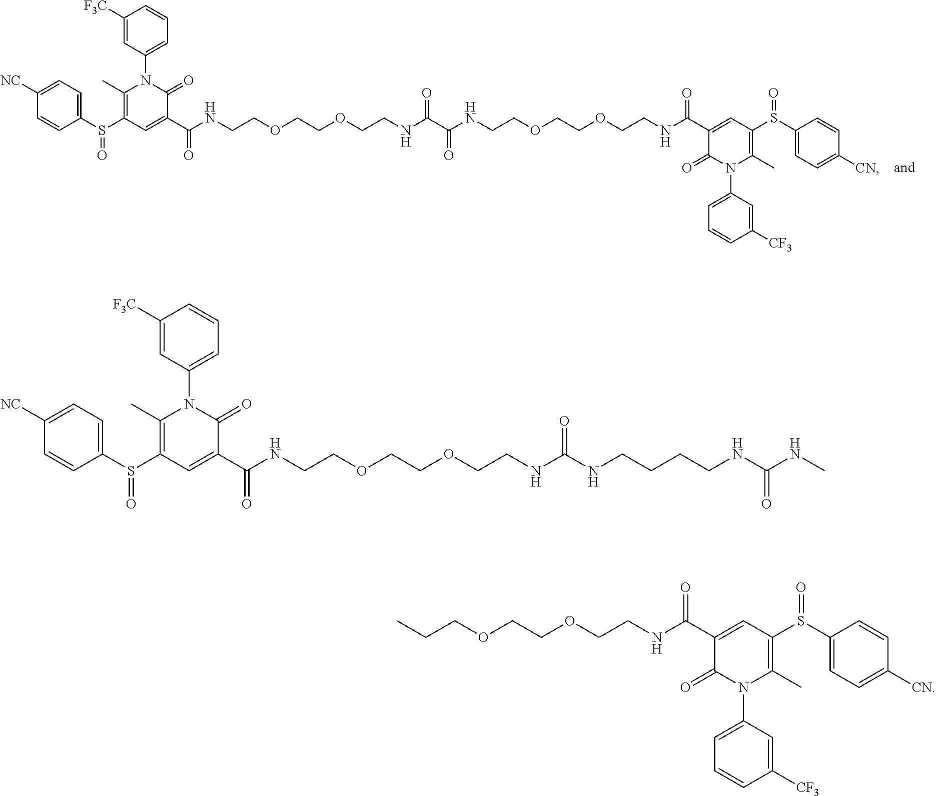

36. A pharmaceutical composition comprising a compound of any one of claims 1-35 or a pharmaceutically acceptable salt, solvate, polymorph, prodrug, metabolite, deuteride, N-oxide, stereoisomer, or isomer thereof, and a pharmaceutically acceptable excipient.

37. A method of treating a lung disease, the method comprising administering a composition comprising a compound of any one of claims 1-35, or a pharmaceutically acceptable salt, solvate, polymorph, prodrug, metabolite, deuteride, N-oxide, stereoisomer, or isomer thereof.

38. The method of claim 37 wherein the lung disease is chronic obstructive pulmonary disease (COPD) or cystic fibrosis.

39. A method of treating an intestinal disease, the method comprising administering a composition comprising a compound of any one of claims 1-35, or a pharmaceutically acceptable salt, solvate, polymorph, prodrug, metabolite, deuteride, N-oxide, stereoisomer, or isomer thereof.

40. The method of claim 39 wherein the intestinal disease is inflammatory bowel disease (IBD).
